(19) United States
(12) Patent Application Publication
(10) Pub. No.: US 2025/0288671 A1
(43) Pub. Date: Sep. 18, 2025

(54) ECM RECEPTOR MODULATION IN NK CELL THERAPY

(71) Applicant: The General Hospital Corporation, Boston, MA (US)

(72) Inventors: Shadmehr Demehri, Charlestown, MA (US); Maulik Vyas, Seattle, WA (US)

(21) Appl. No.: 18/847,026

(22) PCT Filed: Mar. 15, 2023
(86) PCT No.: PCT/US2023/064464

§ 371 Date: Sep. 13, 2024

Related U.S. Application Data
(60) Provisional application No. 63/319,935, filed on Mar. 15, 2022.

(51) Int. Cl. A61K 40/15; A61K 40/31; A61K 40/42; A61P 35/00; C07K 16/28; C12N 5/0783; C12N 9/22; C12N 15/11; C12N 15/86; C12N 15/90

(52) U.S. Cl. CPC A61K 40/15; A61K 40/31; A61K 40/4204; A61P 35/00; C07K 16/2863; C12N 5/0646; C12N 9/226; C12N 15/11; C12N 15/86; C12N 15/907; C12N 2310/20; C12N 2510/00; C12N 2740/15043

(57) ABSTRACT

Provided herein are natural killer (NK) cells with increased, modified or deleted extracellular matrix (ECM) receptors, and methods of making and using the same for cancer immunotherapy, and treatment of chronic infections, inflammation, autoimmune diseases, or transplant rejection.

### CLAIM OF PRIORITY

This application claims the benefit of U.S. Provisional Application Ser. No. 63/319,935, filed on Mar. 15, 2022. The entire contents of the foregoing are incorporated herein by reference.

### TECHNICAL FIELD

Provided herein are natural killer (NK) cells with increased, modified or deleted extracellular matrix (ECM) receptors, and methods of making and using the same for cancer immunotherapy, and treatment of chronic infections, inflammation, autoimmune diseases, or transplant rejection.

### BACKGROUND

It has long been appreciated that downregulation of Major Histocompatibility Complex Class I (MHC-I) is a primary mechanism for immune evasion utilized by cancer cells (1). In the era of cancer immunotherapeutics, MHC-I downregulation has emerged as a major resistance mechanism against immunotherapy as malignant cells escape from CD8⁺ T cell detection and elimination (1). However, the pervasive selection of this immune evasion strategy by cancer cells is perplexing in that MHC-I downregulation is expected to render cells vulnerable to Natural killer (NK) cell killing (2-4). The dilemma of NK cells' role in controlling cells that fail to express self MHC-I in vivo was originally encountered in the transplantation field. In the context of hybrid resistance, parental skin grafts are accepted, but bone marrow (BM) cells are rejected by F1 hybrids in an NK cell-dependent manner (5, 6). Subsequent studies have shown that NK cells in wild-type (WT) syngeneic recipients reject donor MHC-I-deficient BM cells, while MHC-I-deficient solid organ transplants are not rejected (7, 8). Elucidating the mechanism that underlies tissue-specific NK cell responses will have major implications in cancer immunology, transplant biology and virology.

NK cells belong to the innate lymphoid cell (ILC) family that reside in most tissues and form a swift acting innate barrier against viral infections and cells undergoing malignant transformation (9). Conventional NK (cNK) cells, defined by expression of NK1.1, NKp46 and CD49b in mice, are found in circulation, secondary lymphoid organs and most other tissues (9). Another member of the ILC family that is closely related in phenotype and function is the tissue-resident NK (trNK) or ILC1 cell that lacks CD49b but expresses CD49a (9). These cells are a non-migrating, NK cell-like population (10) that are thought to develop from two distinct pathways: 1) differentiation from the innate lymphoid cell progenitor (ILCP) (11) or 2) differentiation from cNK cells in the presence of TGFβ (12, 13) in a non-mutually exclusive manner.

Full NK cell activation against a target cell is accomplished by the integration of multiple inhibitory and activation signals, a process that limits indiscriminate killing of healthy host cells. Self MHIC-I molecules function as the dominant inhibitory signal by binding NK cell-expressed Ly49 receptors in mice, or killer inhibitory receptors (KIR) in humans, to prevent degranulation and cytokine release. Multiple activation signals, together with downregulation or loss of inhibitory signals, are required to enable complete NK cell cytotoxicity (3, 14, 15). Following their licensing to activate their cytotoxic program (16-18), NK cells destroy the target cells by production and release of granzymes and perforin in the immunological synapse (19). Fas-FasL and TRAIL are also used by NK cells for target cell elimination (20).

NK cells expressing chimeric antigen receptors (CAR-NK) are a promising treatment modality for cancers, avoiding many of the issues associated with CAR-T cell therapy.

### SUMMARY

Provided herein are methods of preparing a population of modified Natural Killer (NK) cells. The methods comprise obtaining a first population comprising NK cells; modifying the first population of NK cells to specifically increase, reduce, or eliminate expression of at least one extracellular matrix (ECM) receptor; and optionally maintaining the modified cells in culture under conditions and for a time sufficient for the cells to proliferate, thereby providing a population of modified NK cells.

In some embodiments, modifying the first population of NK cells to specifically increase expression of at least one ECM receptor comprises introducing into the cells at least one expression vector comprising a nucleic acid encoding an ECM receptor polypeptide, optionally wherein the expression vector is a viral vector.

In some embodiments, modifying the first population of NK cells to specifically reduce or eliminate expression of at least one ECM receptor comprises using genome engineering to disrupt at least one ECM receptor gene, optionally wherein the genome engineering used clustered regularly interspaced short palindromic repeats (CRISPR)/CRISPR-associated protein (Cas)-based gene editing, zinc-finger nucleases (ZFN), or transcription activator-like nucleases (TALENs)-based systems to target the ECM receptor gene.

In some embodiments, modifying the first population of NK cells to specifically reduce or eliminate expression of at least one ECM receptor comprises contacting the cells with one or more inhibitory nucleic acids, optionally antisense oligonucleotides, LNAs, RNA interference (RNAi), or shRNA, that specifically inhibits expression of the at least one ECM receptor.

In some embodiments, the at least one ECM receptor is selected from the group consisting of LAIR1, GPR56, ITGB1, ITGA2, ITGAM, ITGB2, ITGA6, ITGA4, NEU1, EBP, ITGB3, PLAUR, DDR1, DDR2, ITGA3, ITGA1, ITGAV, CTSA, LGALS3, and ITGB5, and combinations of two or more thereof. In some embodiments, the at least one ECM receptor is selected from the group consisting of Lair1, GPR56, Integrin α2 and integrin-β, and combinations of two or more thereof.

In some embodiments, the first population of NK cells is obtained from autologous and/or allogeneic peripheral blood mononuclear cells (PBMCs), umbilical cord blood (UCB), embryonic stem cells (ESC), induced pluripotent stem cells (iPSC), or an immortalized NK cell line. In some embodiments, the cell line is NK-92 or a derivative thereof selected from as NKL and NK101; or YT or a derivative thereof selected from HANK-1, KHYG-1, NK-YS, and NKG.

In some embodiments, the cells are modified to specifically reduce or eliminate expression of at least one ECM receptor, and are further modified to express a chimeric antigen receptor (CAR).

Also provided herein are populations of modified NK cells prepared by the methods described herein, and compositions comprising the modified NK cells, optionally wherein the NK cells are frozen or cryopreserved.

Additionally provided herein are populations of modified Natural Killer (NK) cells, wherein the NK cells have increased, reduced or eliminated expression of at least one extracellular matrix (ECM) receptor.

In some embodiments, the NK cells have been modified to specifically increase expression of at least one ECM receptor by introducing into the cells at least one expression vector comprising a nucleic acid encoding an ECM receptor polypeptide, optionally wherein the expression vector is a viral vector.

In some embodiments, the NK cells have been modified to specifically reduce or eliminate expression of at least one ECM receptor using genome engineering to disrupt at least one ECM receptor gene, optionally wherein the genome engineering used clustered regularly interspaced short palindromic repeats (CRISPR)/CRISPR-associated protein (Cas)-based gene editing, zinc-finger nucleases (ZFN), or transcription activator-like nucleases (TALENs)-based systems to target the ECM receptor gene.

In some embodiments, the NK cells have been modified to specifically reduce or eliminate expression of at least one ECM receptor by contacting the cells with one or more inhibitory nucleic acids, optionally antisense oligonucleotides, LNAs, RNA interference (RNAi), or shRNA, that specifically inhibits expression of the at least one ECM receptor.

T In some embodiments, the at least one ECM receptor is selected from the group consisting of LAIR1, GPR56, ITGB1, ITGA2, ITGAM, ITGB2, ITGA6, ITGA4, NEU1, EBP, ITGB3, PLAUR, DDR1, DDR2, ITGA3, ITGA1, ITGAV, CTSA, LGALS3, and ITGB5, and combinations of two or more thereof. In some embodiments, the at least one ECM receptor is selected from the group consisting of Lair1, GPR56, Integrin α2 and integrin-β, and combinations of two or more thereof.

In some embodiments, the modified NK cells were obtained from autologous and/or allogeneic peripheral blood mononuclear cells (PBMCs), umbilical cord blood (UCB), embryonic stem cells (ESC), induced pluripotent stem cells (iPSC), or an immortalized NK cell line. In some embodiments, the cell line is NK-92 or a derivative thereof selected from as NKL and NK101; or YT or a derivative thereof selected from HANK-1, KHYG-1, NK-YS, and NKG.

In some embodiments, the cells are modified to specifically reduce or eliminate expression of at least one ECM receptor, and are further modified to express a chimeric antigen receptor (CAR).

Also provided herein are populations of modified NK cells wherein the cells are modified to specifically reduce or eliminate expression of at least one ECM receptor, for use in a method of treating a subject who has cancer, the method comprising administering to the subject a therapeutically effective amount of the modified NK cells. In some embodiments, the NK cells were obtained from the subject who has cancer.

Also provided herein are methods of treating a subject who has cancer, the method comprising administering to the subject a therapeutically effective amount of modified NK cells as described herein.

In some embodiments, the NK cells were obtained from the subject who has cancer.

In some embodiments, the cancer is colon cancer, ovarian cancer, prostate cancer, lymphoid malignancies, myeloma, renal cell carcinoma, breast cancer, or malignant glioma.

Also provided herein are populations of modified NK cells wherein the cells are modified to specifically increase expression of at least one ECM receptor, and their use in a method of treating a disorder associated with unwanted or abnormal immune activation associated with NK cell activation. In some embodiments, the disorder is an autoimmune disease, chronic inflammatory disease, alloreactive immune response against a transplanted graft, or an alloreactive immune response against a fetus, optionally wherein the chronic inflammatory condition is allergic asthma, atopic dermatitis, or inflammatory bowel disease (IBD); the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, or lymphocytic colitis; or the transplant rejection is rejection of a kidney, lung, heart, liver, limb, skin, or multi-organ transplant.

Additionally provided herein are methods of treating a disorder associated with unwanted or abnormal immune activation associated with NK cell activation, comprising administering a therapeutically effective amount of a population of modified NK cells having increased expression of one or more EMC receptor proteins as described herein, to a subject who is in need of, or who has been determined to be in need of, such treatment. In some embodiments, the disorder is an autoimmune disease, chronic inflammatory disease, alloreactive immune response against a transplanted graft, or an alloreactive immune response against a fetus, optionally wherein the chronic inflammatory condition is allergic asthma, atopic dermatitis, or inflammatory bowel disease (IBD); the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, or lymphocytic colitis; or the transplant rejection is rejection of a kidney, lung, heart, liver, limb, skin, or multi-organ transplant.

Provided herein are methods of inducing NK cell cytotoxicity against cancers comprising administering to the subject an effective dose of NK cell-based therapeutic product with modification of an individual ECM receptor by means of genomic deletion or genetic modification

Additionally provided are methods of inducing NK cell cytotoxicity against cancers comprising administering to the subject an effective dose of NK cell-based therapeutic product with modification of a combination of ECM receptors by means of genomic deletion or genetic modification

Further provided herein are methods of inducing NK cell cytotoxicity against auto-reactive immune cells for the treatment of autoimmune diseases comprising administering to the subject an effective dose of NK cell-based therapeutic product with modification of an individual ECM receptor by means of genomic deletion or genetic modification

Also provided herein are methods of inducing NK cell cytotoxicity against auto-reactive immune cells for the treatment of autoimmune diseases in a subject comprising administering to the subject an effective dose of NK cell-based therapeutic product with modification of a combination of different ECM receptors by means of genomic deletion or genetic modification

Additionally provided herein are methods of inducing NK cell cytotoxicity against inflammatory cells for the treatment of chronic inflammatory diseases in a subject comprising administering an effective dose of NK cell-based therapeutic product with modification of an individual ECM receptor by means of genomic deletion or genetic modification

Also provided herein are methods of inducing NK cell cytotoxicity against inflammatory cells for the treatment of chronic inflammatory diseases comprising administering to the subject an effective dose of NK cell-based therapeutic product with modification of a combination of different ECM receptors by means of genomic deletion or genetic modification

Further provided herein are methods of inducing NK cell regulatory function against inflammatory cells for the treatment of chronic inflammatory diseases, prevent alloreactive immune response against transplanted graft and prevent alloreactive immune response against a fetus for the treatment of pregnancy complications comprising administering to the subject an effective dose of NK cell-based therapeutic product with modification of an individual ECM receptor by means of genomic deletion or genetic modification

Also provided herein are methods of inducing NK cell regulatory function against inflammatory cells for the treatment of chronic inflammatory diseases, prevent alloreactive immune response against transplanted graft and prevent alloreactive immune response against a fetus for the treatment of pregnancy complications comprising administering to the subject an effective dose of NK cell-based therapeutic product with modification of a combination of different ECM receptors by means of genomic deletion or genetic modification

Also provided herein are methods of inducing NK cell helper or direct cytotoxicity function against microorganisms for the treatment of chronic infections comprising administering to the subject an effective dose of NK cell-based therapeutic product with modification of an individual ECM receptor by means of genomic deletion or genetic modification

Additionally provided herein are methods of inducing NK cell helper or direct cytotoxicity function against microorganisms for the treatment of chronic infections comprising administering to the subject an effective dose of NK cell-based therapeutic product with modification of a combination of different ECM receptors by means of genomic deletion or genetic modification.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-G. Presence of NK cell activating m157 ligand and loss of inhibitory B2m are insufficient to induce skin graft rejection in syngeneic recipients.
(A) Schematic diagram of ear skin transplantation from B6 albino WT, B2m^{-/-}, m157^{tg} and m157^{tg}, B2m^{-/-} donor mice to B6 Ncr1^{iCre}, ROSA^{mT-mG} recipients.
(B) Representative images of skin grafts at day 20 post-transplant.
(C) Quantification of graft rejection grades assessed at day 20 post-transplant of B6 albino WT, B2m^{-/-}, m157^{tg} and m157^{tg}, B2m^{-/-} skin grafts transplanted onto B6 Ncr1^{iCre}, ROSA^{mT-mG} recipients.
(D) The percentage change in skin graft size from day 10 to day 20 post-transplant.
(E) Representative IF images of NKp46-GFP⁺, CD4⁺ and CD8⁺ cells in skin grafts at day 20 post-transplant. Dotted lines indicate the epidermal basement membrane.
(F) Quantification of NKp46-GFP⁺ NK, CD4⁺ T and CD8⁺ T cells in the skin and epidermis of skin grafts at day 20 post-transplant. (B-F) n=9-12 mice per group from nine independent experiments.
(G) Time-course of skin graft maintenance from day 10 to day 20 post-transplant comparing m157^{tg}, m157^{tg}, B2m^{-/-}, mOVA^{tg} and mOVA^{tg}, B2m skin grafts. m157^{tg} and m157^{tg}, B2m^{-/-} images from the same experiment as (B). mOVA^{tg} and mOVA^{tg}, B2m^{-/-} n=2-5 from one to two independent experiments.
(C) Fisher's exact test. ns: not significant, * p<0.05, *** p<0.001 compared with WT group. (D and F) Graphs show mean±SD. Mann-Whitney Utest, ns: not significant, * p<0.05, **p<0.01, ***p<0.001. (B and G) Scale bars=1 cm, (E) scale bar=100 μm.

Photo Credit: Mark D. Bunting, Massachusetts General Hospital

FIGS. 2A-E. NK cells fail to induce skin graft rejection upon cytokine stimulation.
(A, B) Representative images of WT or m157^{tg} skin grafts treated with poly(I:C)/IL-15/IL-18 (A) and m157^{tg}, B2m^{-/-} skin grafts treated with IL-12/IL-15/IL-18 (B) on WT B6 recipients at day 21 post-transplant. n=2-5 mice per group from one to three independent experiments.
(C) Representative flow cytometry dot plots of NK cells gated as NKp46-GFP⁺ ROSA-TdTomato (ROSA)⁻CD3⁻CD4⁻CD8⁻ in WT and m157^{tg}, B2m^{-/-} skin grafts. cNK, dpNK and trNK were distinguished based on CD49a and CD49b expression. Numbers on the dot plots represent the percent cells within each gate.
(D) Enumeration of cNK, dpNK and trNK populations in the skin by combining flow cytometry frequency data with NKp46-GFP⁺ cell counts from FIG. 1. n=9-10 mice per group from nine independent experiments.
(E) Ly49H expression by cNK, dpNK and trNK cells from m157^{tg}, B2m^{-/-} and cNK cells from WT skin grafts at day 20 post-transplant compared with cNK cells from the liver. Note that WT skin grafts contain Ly49H⁺ and Ly49H⁻ cNK cells while all NK subsets in m157^{tg}, B2m^{-/-} skin grafts express low levels of Ly49H. Average±SD of mean fluorescent intensity (MFI) of Ly49H on NK cell subsets in m157^{tg}, B2m^{-/-} skin grafts and Ly49H⁻ cNK cells in WT Skin grafts at day 20 post-transplant are compared with Ly49H⁻ cNK cells in the liver of the recipient mice. n=4-10 mice per group from three independent experiments.
(D, E) Graphs shown mean±SD. Mann-Whitney U test, ns: not significant, * p<0.05, ** p<0.01, * p<0.001. (A, B) Scale bars=1 cm.

Photo Credit: Mark D. Bunting, Massachusetts General Hospital

FIGS. 3A-E. Circulating cNK cells enter the skin grafts and give rise to trNK cells.
(A) Schematic of parabiosis between WT and Ncr1^{iCre}, ROSA^{mT-mG} B6 mice followed by m157^{tg}, B2m^{-/-} skin transplantation onto the WT parabiont. Representative dot plots of the spleen and donor skin from the WT parabiont gating on CD3⁻CD4⁻CD8⁻ cells and showing CD45 I.V. and NKp46-GFP at day 20 post-skin transplant.
(B) Representative IF images of m157^{tg}, B2m^{-/-} skin graft from the WT parabiont showing the presence of NKp46-GFP⁺ cells (green, white arrows), CD4⁺ and CD8⁺ cells. Dotted lines indicate the epidermal basement membrane. n=5 parabiosis pairs.
(C) Sorted splenic cNK cells from Ncr1^{iCre}, ROSA^{mT-mG} mice were adoptively transferred into WT mice one day after skin transplantation with m157^{tg}, B2m^{-/-} skin grafts. Representative dot plots of transferred NK cells (defined as CD3-CD4-CD8-NKp46-GFP⁺) in liver and skin grafts of mice that did not (left) and did (right) receive NKp46-GFP⁺ NK cells. n=2 mice per +NK group from two independent experiments.
(D) Representative images of m157^{tg}, B2m^{-/-} skin grafts transplanted onto WT, Ncr1^{iCre}, Tgfbr2^{fl/fl} and Hobit^{-/-} recipient mice at day 20 post-transplant. n=4-7 mice per group.
(E) Frequencies of cNK and trNK cells in the recipient liver (top) and donor skin (bottom) at day 20 post-transplant. Ncr1^{iCre}, ROSA^{mT-mG} mice transplanted with B6 albino WT, m157^{tg}, B2m^{-/-} and m157^{tg}, B2m^{-/-} donor skin from FIG. 1 (n=9-10 mice per group) and Ncr1^{iCre} Tgfbr2^{fl/fl} and Hobit^{-/-} recipients transplanted with m157^{tg}, B2m^{-/-} donor skin at day 20 post-transplant (n=4-7 mice per group).
(E) Graphs shown mean±SD. Mann-Whitney U test, * p<0.05, **p<0.01.
(B) scale bar=100 m, (D) Scale bars=1 cm.

Photo Credit: Mark D. Bunting, Massachusetts General Hospital

FIGS. 4A-G. Presence of activating m157 and loss of self-MHC induces robust skin graft rejection in allogeneic recipients.
(A) Schematic of ear skin transplant from B6 albino WT and m157^{tg} donor mice to F1 B6×BALB/c Ncr1^{iCre}, ROSA^{mT-mG} recipients.
(B) Representative images of skin grafts at day 12, 20 and 60 post-transplant.
(C) The percentage change in skin graft size from day 10 to day 20 post-transplant. n=4-8 mice per group from two independent experiments.
(D) Representative IF images of NKp46-GFP⁺, CD4⁺ and CD8⁺ cells in skin grafts at day 10 post-transplant. Dotted lines indicate the epidermal basement membrane.
(E) Quantification of NKp46-GFP⁺ NK, CD4⁺ T and CD8⁺ T cells in the skin grafts at days 3, 5, 7 and 10 post-transplant. Red arrow highlights NK cells infiltrating the skin prior to T cells. n=3-9 mice per group from one to three independent experiments.
(F) Comparison of m157^{tg} skin graft rejection in control (ROSA^{DTR}+DT/control IgG), NK cell-depleted (Ncr1^{iCre}, ROSA^{DTR}+DT/anti-NK1.1 antibody) and T cell-depleted (CD4^{Cre}, ROSA^{DTR}+DT/anti-CD4/8 antibodies) F1 mice at day 20 post-transplant.
(G) Representative images of m157^{tg} skin graft rejection in NK cell-depleted mice at day 35 and 60 post-transplant following cessation of NK cell depletion at day 17 post-transplant.

(B, D, F, G) n=4-8 mice per group from two independent experiments. (C and E) Graphs show mean±SD, Mann-Whitney Utest, ns: not significant, * p<0.05, ** p<0.01, *** p<0.001. (B, F, G) Scale bars=1 cm, (D) scale bar=100 μm.

Photo Credit: Mark D. Bunting and Marta Requesens, Massachusetts General Hospital.

FIGS. 5A-C. cNK cells' transcriptome changes dramatically as they emigrate from the circulation into ECM-rich skin grafts.
(A) Comparison of up- and down-regulated genes in circulating cNK cells, m157-expressing donor skin-derived cNK, dpNK, and trNK cells from donor and recipient skin as determined by RNA-Seq analysis of sorted NK cell subpopulations. n=6 mice for spleen, blood, cNK cell in donor skin and trNK cells in recipient skin, n=3 mice for donor dpNK and trNK cells in donor skin. Note that three F1 recipients with m157^{tg} skin graft and three B6 recipients with m157^{tg}, B2m^{-/-} skin graft were included. NK cell sorting strategy is shown in fig. S6A.
(B) Gene expression changes in donor skin-derived cNK cells compared with circulating cNK cells in the spleen and blood.
(C) Representative images of NKp46-GFP⁺ NK cells, vimentin⁺ fibroblasts and collagen/elastin-stained adjacent sections of m157^{tg} skin graft at day 5 post-transplant onto F1 recipient mice. DAPI marks the cell nuclei. n=7 from two independent experiments. Dotted lines indicate the epidermal basement membrane. Scale bars=100 μm, insets=50 μm.

FIGS. 6A-M. Dermal ECM proteins modulate cNK cell effector function in vitro.

(A, B) Quantification of CD107a (A) and IFNγ (B) by Ly49H⁺ splenic cNK cells after 7 hours co-culture with no MEF, WT-MEF or m157-MEF in the presence of IL-12 and IL-15. n=5 per group.
(C) Representative dot plots of CD107a by splenic cNK cells co-cultured with WT-MEF or m157-MEF. Representative histograms of Ly49H expression by Ly49H⁺CD107a⁺ NK cells following co-culture with m157-MEF and WT-MEF. Histograms of Ly49H⁺CD107a⁻ cNK cells (black) are shown as controls. Numbers on the dot plots represent the percent cells within each gate.
(D-G) Quantification of CD107a (D, E) and IFNγ (F, G) expression by Ly49H⁺ splenic cNK cells co-cultured with different ECM proteins and m157-MEF at 9 hours (D, F) and 24 hours (E, G). Percentages are normalized based on the “No ECM” group. n=7 per group from two independent experiments.
(H, I) Comparison of CD107a (H) and IFNγ (I) by Ly49H⁺ splenic cNK cells from WT and Lair1^{-/-} mice in the presence of m157-MEF and collagen I. Percentages are normalized based on “No ECM” group in WT (n=7) and Lair1^{-/-} (n=12) experiments.
(J-M) Measurement of CCL2 (J), CXCL10 (K), CCL5 (L) and CXCL9 (M) in culture media of splenic cNK cells co-cultured with m157-MEF in the presence of no ECM, collagen I, III or elastin at 12 hours (CXCL10, CCL5) and 24 hours (CCL2, CXCL9). n=8 per group from two independent experiments.
(A, B, D-M) Graphs show mean±SD. Mann-Whitney U test, ns: not significant, * p<0.05, ** p<0.01, ***p<0.001 compared with no ECM group.

FIGS. 7A-I. Collagens and elastin modulate cytotoxicity and inflammation-associated signaling pathways in NK cells.
(A) GSEA enrichment plots of PI3K-AKT-mTOR, TNFα-NF□DB, IL-6-JACK-STAT3 and IL-2-STAT5 signaling in CD49b⁺ cNK cells in the donor skin compared with the circulation. The enrichment scores (ES) and P values are indicated in each plot.
(B) Quantification of phospho(p)NF□DB (p65) in splenic cNK cells at 30 and 60 minutes post-coculture with m157-MEF in the presence of different ECM proteins. n=4 per group, data representative of two independent experiments.
(C-G) Quantification of phospho(p)PLCγ1⁺ (C), pERK1/2⁺ (D), pNF□B (p65)⁺ (E), pSTAT3⁺ (F) and pSTAT5⁺ (G) in splenic cNK cells at 2- and 5-minutes post-stimulation with H₂O₂ in the presence of different ECM proteins. n=8 per group from two independent experiments. Representative flow cytometry histograms comparing the phosphorylation of key signaling proteins at 2- and 5-minutes post-stimulation with H₂O₂ in response to no ECM (gray), collagen I, collagen III (orange) and elastin. Black dotted line corresponds to the gating based on the “no ECM−0 min” histogram for each signaling protein.
(H, I) Comparison of pERK1/2 (H) and pNF□DB (p65) (I) in splenic NK cells from WT and Lair1^{-/-} mice in response to 2 minutes H₂O₂ stimulation in the presence of collagen I. Percentages are normalized based on “no ECM” group in WT and Lair1^{-/-} experiments. n=4 per group, data is representative of two independent experiments.
(B-I) Graphs show mean±SD. Mann-Whitney U test, ns: not significant, * p<0.05, ** p<0.01, *** p<0.001. (C-G) each time point is compared with the corresponding time point in no ECM group.

FIGS. 8A-L. Collagen deposition blocks NK cells from eliminating B2m-deficient melanoma cells in the skin.
(A, B) B2m^{-/-} B16 melanoma S.C. growth over time (A) and terminal weight (B) in WT mice treated with control IgG (n=17 for (A) and 13 for (B)) or anti-NK1.1 antibody (n=19 for (A) and n=13 for (B)).
(C) Representative images of NKp46-GFP⁺ NK cells (white arrows) immunofluorescence and collagen stain in adjacent sections of a B2m^{-/-} B16 melanoma at day 3 post-S.C. injection in Ncr1^{iCre}, ROSA^{mT-mG} mice.
(D-G) B2m^{-/-} B16 melanoma S.C. growth over time (D, F) and terminal weight (E, G) in WT mice treated with Losartan (D, E) or DHB (F, G) plus control IgG (n=28 (D), 20 (F), 27 (E) and 20 (G)) or anti-NK1.1 antibody (n=26 (D), 20 (F), 25 (E) and 18 (G)).
(H, I) Representative images (H) and quantification (I) of NKp46-GFP⁺ NK cells in B2m^{-/-} B16 melanoma at day 3 post-S.C. injection in PBS (n=10), Losartan (n=7) and DHB (n=9) treated Ncr1^{iCre}, ROSA^{mT-mG} mice.
(J-L) Quantification of Granzyme B (J), IFNγ production (K) and PD-1 surface expression (L) on NK cells isolated from B2m^{-/-} B16 melanoma at day 19-21 post-S.C. injection in PBS (n=13 (J, L), 8 (K)), Losartan (n=21 (J), 22 (K, L)) and DHB (n=20 (J, K, L)) treated mice.
(A, D, F) Two-way ANOVA, (B, E, G, I-L) Graphs show mean±SD, Mann-Whitney Utest. ns: not significant, * p<0.05, ** p<0.01, *** p<0.001. (C and H) Scale bars=100 μm.

FIG. 9. Schematic diagram of ECM protein-mediated switch in cNK cell effector function in the skin. Created with BioRender.com

FIG. 10. Grading system to score skin graft rejection. Scale bar=1 cm.

FIGS. 11A-G. NK cell sorting strategy for RNA-Seq, GSEA plots demonstrating the switch in cNK cell phenotype as they enter the skin, differential chemokine/cytokine receptor expression in NK cell subsets and the effect of immune checkpoint blockade on skin graft rejection outcomes.
(A) Sorting strategy for cNK, dpNK and trNK cells isolation from m157tg-expressing skin grafts transplanted onto Ncr1iCre, ROSAmT-mG mice. Note that the recipient blood and spleen only had CD45 I.V.+NK cells, which were CD49b⁺ cNK cells, and CD45 I.V.−NK cells in the recipient skin were CD49a+ trNK cells.
(B) Gene Set Enrichment Analysis (GSEA) enrichment plots for NK cell-mediated cytotoxicity and inflammatory response in CD49b⁺ cNK cells isolated from the donor skin compared with the circulation. The enrichment scores (ES) and P values are indicated in each plot.
(C) Differential expression of cytokine and chemokine receptors as determined by RNA-Seq in circulating and donor skin-derived NK cells.
(D) Representative flow cytometry histograms comparing surface expression of TGFβRII, TIGIT, IL-4Rα and CCR7 on peripheral blood cNK cells, donor skin cNK cells and donor skin trNK cells. Gating was set based on FMO control.
(E) Schematic diagram of immune checkpoint blockade treatment strategy used in transplanted animals.
(F) Representative images of m157tg, B2m−/− skin grafts on WT B6 recipients treated with control IgG or blocking antibodies against PD-1 or TIGIT. Graft rejection was assessed at day 20 post-transplant. n=2-4 mice per group from two independent experiments.
(G) Representative images of m157tg skin grafts on WT B6 at day 20 or F1 recipients at day 10 post-transplant treated with control IgG or anti-TIGIT/PD-1/CTLA-4 combination blocking antibodies. n=2-4 mice per group from two independent experiments.
(F, G) Scale bars=1 cm.

Photo Credit: Marta Requesens, Massachusetts General Hospital

FIGS. 12A-C. Skin graft infiltrating NK cells are embedded within collagens and elastin in the dermis and Lair1−/− mice fail to reject m157tg, B2m−/− skin grafts.
(A) Representative IF image of NKp46-GFP and collagen/elastin stain on adjacent sections of m157^{tg} skin graft at day 10 post-transplant onto F1 recipient mice. n=9 from two independent experiments. DAPI marks the cell nuclei. Scale bar=100 m, inset=50 μm.
(B) Representative IF image of NKp46-GFP and vimentin in m157^{tg} skin grafts at day 5 posttransplant onto F1 recipient mice. Arrow highlights NKp46-GFP⁺ NK cell contacting vimentin+dermal fibroblast. n=7 from two independent experiments. Scale bar=50 μm.
(C) Representative images of m157tg, B2m−/− skin grafts on Lair1−/− B6 at day 20 post-transplant. n=2-3 mice per experiment from two independent experiments (scale bar=1 cm).

Photo Credit: Maulik Vyas and Marta Requesens, Massachusetts General Hospital.

FIG. 13. NK cells express a wide variety of ECM receptors that are altered upon entry into the skin graft.

Differential expression of ECM receptor genes by circulating cNK cells, m157-expressing donor skin-derived cNK and dpNK cells, and trNK cells from donor and recipient skin as determined by RNA-Seq analysis of sorted NK cell subpopulations. n=6 mice for cNK cell and recipient skin trNK cell samples, n=3 mice for donor skin dpNK and trNK cells. NK cell sorting strategy is shown in FIG. 11A.

FIGS. 14A-C. B16 melanoma infiltrating NK cells are found to interact with collagen within the tumor stroma.
(A) Schematic diagram of WT and B2m^{-/-} B16 melanoma cell inoculation and anti-NK1.1 antibody treatment for the subcutaneous (S.C.) melanoma model. Note: Losartan, DHB or PBS (carrier) treatments were given to mice receiving B2m^{-/-} B16 melanoma cells.
(B) WT B16 melanoma subcutaneous (S.C.) growth rate over time in WT mice treated with control IgG (n=17 for tumor growth) or anti-NK1.1 antibody (n=19 for tumor growth). Two-way ANOVA, ns: not significant.
(C) Representative images of B2m^{-/-} melanoma center (left) and periphery field of view (right) showing NKp46-GFP⁺ cells (white arrows) and collagen stain of the adjacent tumor section at day 20 postinjection. DAPI marks the cell nuclei. Scale bars=100 μm.

FIGS. 15A-L. Inhibition of collagen deposition leads to NK cell-mediated control of B2m−/− melanoma in the skin.
(A, B) B2m−/− B16 melanoma S.C. growth over time (A) and terminal weight (B) in WT mice treated with PBS (gray; n=28 (A), 24 (B)), losartan (orange; n=28 (A), 27 (B)) or DHB (purple; n=20 (A and B)). Note that all animals received control IgG and the data are also shown in FIGS. 7, A, D and F (IgG groups).
(C) Kaplan-Meier survival plot for WT mice treated with PBS (gray; n=28), Losartan (orange; n=28) and DHB (purple; n=20) following subcutaneous injection of B2m−/− B16 melanoma cells. Note that all animals received control IgG and tumor growth from these mice is shown in (A). Mice were considered moribund when tumor volume reached 1 cm3.
(D) B2m−/− B16 melanoma S.C. growth over time in WT mice treated with anti-NK1.1 antibody plus PBS (magenta; n=19), losartan (green; n=26) or DHB (blue; n=20). Note that the data are also shown in FIGS. 7, A, D and F (αNK1.1 groups).
(E, F) Representative images (E) and quantification (F) of CD3+ T cells in B2m−/− B16 melanoma at day 3 post S.C. injection in PBS (n=9), Losartan (n=9) and DHB (n=9) treated Ncr1iCre, RosamT-mG mice. (E) Scale bar=100 μm.
(G) Proportion of CD3+CD4+Foxp3+ regulatory T cells (Tregs) in B2m^{-/-} B16 melanoma from WT mice treated with PBS (n=17), Losartan (n=29) and DHB (n=20) examined by flow cytometry at the endpoint.
(H-J) Proportion of CD11b+ (H), F4/80+(I) and Arginasel+ (J) cells in B2m−/− B16 melanoma from WT mice treated with PBS (n=9), Losartan (n=7) and DHB (n=9) examined by immunofluorescence staining at the endpoint.
(K) B2m−/− B16 melanoma S.C. terminal weight in WT mice treated with collagenase and hyaluronidase plus control IgG (n=24) or anti-NK1.1 antibody (n=18).
(L) Scoring S.C. B2m−/− B16 melanoma metastasis to the draining lymph nodes in WT mice treated with PBS, losartan or DHB and received control IgG versus anti-NK1.1 antibody. PBS: IgG (n=8) and anti-NK1.1 (n=8); losartan: IgG (n=19) and anti-NK1.1 (n=23); DHB: IgG (n=15) and anti-NK1.1 (n=18), Fisher's exact test.
(A, D) Two-way ANOVA, ns: not significant, * p<0.05, ** p<0.01, *** p<0.001. (C) Log-rank (Mantel-Cox) test. (B, F-K) graphs show mean±SD. Mann-Whitney Utest, ns: not significant, * p<0.05, ** p<0.01, ***p<0.001.

Photo Credit: Marta Requesens, Massachusetts General Hospital

FIGS. 16A-E. Impact of NK cells on WT and B2m^{-/-} melanoma lung metastases.
(A) Schematic diagram of WT and B2m^{-/-} B16 melanoma cell inoculation and anti-NK1.1 antibody treatment for the metastatic (I.V.) melanoma model.
(B) Quantification of WT B16 melanoma metastatic foci in the lung following tail vein I.V. injection and treatment with control IgG versus anti-NK1.1 antibody. n=10 mice per group.
(C) Quantification of B2m^{-/-} B16 melanoma metastatic foci in the lung following tail vein I.V. injection and treatment with control IgG versus anti-NK1.1 antibody. n=10-15 mice per group.
(D, E) Representative macroscopic images of lung metastases of WT B16 (D) and B2m−/− B16 (E) at day 14 post-I.V. injection. Antibody treatments are listed below lung images.
(B, C) Graphs show mean+SD. Mann-Whitney Utest, ns: not significant, ** p<0.01, *** p<0.001, compared with IgG-treated group.

Photo Credit: Maulik Vyas, Massachusetts General Hospital

FIGS. 17A-F. B2M gene alterations in solid versus blood cancers in humans.
(A) B2Mgene alteration frequency in various solid cancers and leukemias from the TCGA pan-cancer dataset and other public cancer databases from eBioPortal.
(B) Quantification of B2M expression variance in the TCGA RNA-Seq expression dataset among several solid cancer types and AML. Line highlights the floor of B2M expression in AML.
(C-F) The distribution of mutational burden (number of mutations) in leukemias and various solid cancer samples from the TCGA pan-cancer dataset and other public cancer databases from cBioPortal, and the mutational burden of solid cancer samples that contain B2M deletion or mutation. Although B2M deletion was not found in leukemias, B2M deletion and mutation were readily detectable across solid cancer types even in those with low mutational burden (arrows).

FIGS. 18A-B. ECM protein-receptor interactions modulate mouse and human NK cell function.
(A-B) MDA-MB-231 (target) cell killing by EGFR-CAR NK92 or un-transduced NK92 cells (A) and by EGFR-CAR NK92 cells in 3D collagen-I matrix and pre-incubated with Lair1 blocking or IgG control antibody (B). 1:1 effector to target ratio was used to assess the target cell death by 7AAD uptake. N=3-5 per group, * P<0.05, ** P<0.01, NS: not significant, Mann-Whitney Utest.

FIG. 19. Format of exemplary CAR constructs for CAR-NK cells.

As described in detail below, “anti-EGFR scFv” moiety in the exemplary EGFR construct can be replaced with one or more scFv to achieve mono or multiple specificity for given antigens on target cells. This exemplary CAR construct contains 4-1BB co-stimulatory and CD3 signaling domains. Multiple signaling, co-stimulatory and co-inhibitory domains have been reported that can be used in this prototype construct to generate CAR constructs with desired effector function.

### DETAILED DESCRIPTION

Although in vitro models of NK cell activation have provided insights into signaling requirements that initiate NK cell killing of target cells, the conditions in vivo are much more complex in that non-MHC ligands and potentially unknown inhibitory and activation pathways of varying strengths, acting together with cytokines, set the NK cell activation threshold (21). Most in vivo NK cell activation models have focused on NKG2D and other activating ligands and cytokines that also activate CD8⁺ T cells (22), complicating interpretation of NK versus CD8⁺ T cell effects. To investigate the mechanism of NK cell activation in peripheral tissues, we utilized the NK cell-specific m157-Ly49H activation axis in a skin transplantation model. m157 is murine cytomegalovirus (MCMV)-encoded glycoprotein expressed on the surface of infected cells. This MHC-I-like molecule serves as an NK cell-specific activation signal, directing the targeted killing of MCMV infected cells through recognition by the Ly49H receptor, which is only present on the surface of NK cells on the C57BL/6 (B6) background (23). As a result, B6 mice are resistant to MCMV infection, while BALB/c and other strains are susceptible (24). Thus, the m157-Ly49H axis can be used to specifically study NK cell activation and its downstream function in vivo.

Large numbers of NK cells are recruited into m157-expressing donor skin; however, NK cells are unable to directly reject the skin grafts even with the loss of MHC-I and the addition of NK cell-stimulating poly(I.C) and cytokines in a syngeneic model. This demonstrates that the MHC-I-independent inhibition of NK cell's cytotoxicity dominated over multiple strong activation signals tested. Skin graft rejection is accomplished using F1 recipients, which provides missing-self for NK cell activation while simultaneously preserving MIC-I expression on the donor cells. In this system, NK cells licensed by BALB/c MIC-I are fully activated against B6 MHC-I-expressing m157⁺ donor skin cells (i.e., missing-self) (25) while CD8⁺ T cells are able to contribute to the rejection as the MHC-I signaling axis remains operational. Graft rejection begins after day 12 post-transplant, which is approximately 7 days later than the initial accumulation of NK cells in the graft. The majority of rejection is completed by day 20 post-transplant, which is in agreement with the timing of an adaptive immune response. The timing of NK and T cell infiltration into the skin graft, the kinetics of graft rejection and the requirement for both NK and T cells in this process suggest that NK cells activate the adaptive immune response and T cells function as effectors to reject m157′⁹ donor skin grafts in F1 recipients.

As demonstrated herein, upon exit from the circulation into the skin graft in response to m157, NK cells are exposed to extracellular matrix (ECM) proteins, which can block NK cell cytotoxicity while promoting helper function (FIG. 9). NK cell activation induced by the combination of m157 and missing self MHC-I (25) resulted in skin graft rejection in an NK and T cell-dependent manner. RNA-Seq analysis revealed significant downregulation in cytotoxic mediators together with upregulation in inflammatory cytokines and chemokines, when comparing cNK cells that entered the skin with circulating cNK cells. We found that in vitro exposure to ECM components, collagens and elastin, transformed the function and signaling within cNK cells. Furthermore, control of circulating melanoma cells occurred in an NK cell-dependent manner, yet the growth of these same melanoma cells subcutaneously was not impacted by NK cells embedded in ECM. Importantly, blocking collagen deposition in subcutaneous melanomas led to an NK cell-mediated tumor suppression. Human solid cancers, but not leukemias, could afford to downregulate MHC-I to escape CD8⁺ T cell-mediated elimination, which suggested that NK cells within peripheral tissues, unlike those in circulation, lacked a direct cytotoxic function. Our study reveals a fundamental aspect of NK cell biology, which governs the interplay of NK cells with cancer, organ transplantation and viral infection.

Our data strongly suggest that the interplay between several ECM proteins and NK cell receptors play a cooperative role in this functional shift immediately upon entry of cNK cells into the skin. NK cell-ECM protein interactions suppress NK cell's direct cytotoxicity and instead promote its helper function to recruit and activate adaptive immunity. Although this regulation of NK cell cytotoxicity may limit nonspecific tissue damage in the context of localized viral infection in peripheral tissues, it allows the outgrowth of cancer cells that have managed to evade cytotoxic T cell immunity.

Previous studies have shown a role for TGFβ in impairing NK cell function in cancer models by promoting the conversion of cNK cells to trNK cells (12, 13, 37). We show the impairment in NK cell cytotoxicity is an immediate consequence of cNK cell exit from the circulation into a peripheral tissue microenvironment. In response to skin transplantation, CD49a⁺CD49b⁻ trNK cells develop from cNK cells that have exited the circulation and populated the skin graft over time. Interestingly, we find that cNK cells upregulate TGFβ receptor as soon as they exit circulation, which may promote their conversion into trNK cells in the TGFβ-rich environment of the skin graft. Although they may provide cytokine-mediated help to T cells later in the response, trNK cells are not seen in significant numbers until after day 14 post-transplant. In addition, blocking trNK cell development by deleting Tgfbr2 (12, 13) or Hobit transcription factor (38) does not result in NK cell cytotoxicity or graft rejection. These findings demonstrate that the switch in NK cell function from killer to helper is an immediate consequence of its exit from circulation and entry into the peripheral tissue microenvironment.

Cancers in peripheral tissues that are expected to be strong targets for NK cells have often failed to show objective responses (57-59). The present results demonstrate that NK cells experience inhibition of their cytotoxicity likely mediated by collagens and elastin once they exit the circulation and enter the stroma. This may explain the distinct selection for loss of MHC-I by solid cancers but not leukemias in humans. We speculate that NK cell's rapid cytotoxic response in the circulation and delayed helper response in tissues can be explained by selection pressure to prolong host survival. The suppression of a cytotoxic innate immune response in the peripheral tissues may prevent over-reaction to localized pathological insults, which could predispose to excessive tissue damage and the development of chronic inflammation. Meanwhile, a “helper” immune response for the development of an overall more targeted, strength-appropriate adaptive immunity may be best suited to combat viral infections in peripheral tissues. In contrast, infection of the blood requires immediate control to ensure host survival. These evolutionary choices may explain the switch in NK cell function as they exit circulation and enter peripheral tissues.

### Modified NK Cells—Overexpressing or Lacking One or More ECM Receptors

Provided herein are methods and compositions that use NK cells modified by overexpression, knockout or knockdown of one or more ECM receptors. NK cells modified to overexpress one or more ECM receptors can be used to treat or reduce risk of graft rejection or infections (e.g., viral infections), while NK cells modified to have reduced or eliminated expression of one or more ECM receptors can be used to enhance anti-tumor activity, especially against solid tumors. The NK cells can be untreated, pre-treated with cytokines (e.g., IL-12, IL-15 and IL-18) and/or further modified to express chimeric antigen receptor (CAR, e.g., CAR-NK cells), Bi- and tri-specific killer engagers, BiKEs and TriKEs (e.g., CD16/IL-15/CD33 TriKE, GTB-3550), or Tri-functional NK cell engagers (NKCEs) that crosslink both NKp46 and CD16; see, e.g., Liu et al., Journal of Hematology & Oncology 14, Article number: 7 (2021). The methods and compositions can use autologous or allogeneic NK cells, which are modified using genetic engineering methods or inhibitory nucleic acids to delete or reduce expression or function of one or more ECM receptor proteins, e.g., as listed in Table 1, e.g., Lair1, GPR56, Integrin α2 and integrin-β. The compositions can include NK cell-based therapeutic products, such as CAR NK cells, allogeneic and autologous NK cells for adoptive transfer, iPSC-derived NK cells, and NK cell line products.

### NK Cells

NK cells used in the present methods and compositions can be obtained from a number of sources. For example, patients' and/or donors' peripheral blood mononuclear cells (PBMC) can be used as an autologous and/or allogeneic source of NK cells, respectively (61). Further allogeneic NK cell sources include umbilical cord blood (UCB), placental blood, embryonic stem cells (ESC), induced-pluripotent stem cells (iPSC), multipotent progenitor cells (CD34⁺ hematopoietic stem cells) (61). Thus the cells can be derived from healthy donor derived peripheral blood, induced pluripotent stem cells (iPSC), umbilical cord stem cells, oNKord cells (allogeneic partial HLA-matched NK cells derived from UCB-CD34+ progenitors), placenta-expanded NK cells (CYNK-001), CTV-1 lysate-primed human NK cells (CNDO-109-NK cells), or other natural sources; or modified immortalized cells, NK-92 cells, NK-101 cells, or other NK cells obtained and expanded from patients with NK lymphomas, or variants of each of these. Immortalized NK cell lines are alternative source of NK cells. Eight malignant NK cell lines that are established include NK-92 and its derivatives, NKL, NK101, YT and derivatives, HANK-1, KHYG-1, NK-YS and NKG (61). NK-92, established from the blood of 50-year old male patient with non-Hodgkin lymphoma, is FDA approved NK cell line for the clinical testing as an adoptive NK cell therapy (62). Freshly isolated and/or expanded NK cells are traditionally used as therapeutic products but recent reports describe the successful application of cryopreserved NK cells or NK cell source as starting material. When using primary NK cells as source for CAR-NK cells, expansion of NK cells ex vivo is necessary to achieve clinically useful amount of NK cells. Several methods have been described to achieve the robust expansion of NK cells (63). Most widely-used strategies include cytokine-based (IL-2, IL-12, IL-15, IL-18 and/or IL-21) expansion, K562-feeder cell based (K562-IL-15/IL-21/4-1BBL) expansion, autologous PBMC stimulated cells as feeder cells to expand NK cells and synthetic agonist based expansion (63). A brief exposure to a combination of IL-12, IL-15 and IL-18 results in the generation of cytokine-induced memory like NK cells with enhanced effector functions (64). Serum or feeder-free iPSC derived NK cell generation is already established and this differentiation has been shown to reproducibly generate clinical-scale mature NK cells in just 5 days (65). Based on the positive preclinical outcome of iPSC-derived NK cells, the FDA has approved a phase I clinical trial to investigate the iPSC-NK product, FT50 (65).

The modified NK cells described herein can be provided, e.g., freshly isolated and/or expanded from primary cells from a subject, from cell lines, or can be cryopreserved using methods known in the art (84, 85). Thus provided herein are compositions comprising the modified NK cells wherein the cells are cryopreserved or are present in cell-supportive media or physiologically acceptable buffer.

### CAR-NK Constructs

The adoptive therapy of NK cells takes advantage of inherent cytotoxic potential of NK cells that is even more pronounced in allogeneic setting due to HLA and KIR mismatch between the donor and recipient. Various genetic modifications have been incorporated in NK cells to overcome some of the limitations faced by these NK cell adoptive transfer therapy in cancer and other diseases. As described herein, modifying ECM receptor on NK cell therapies can improve their efficacy and aims to lower the side effects. One such genetic modification that is most commonly used is to express chimeric antigen receptor (CAR) on NK cells. CAR constructs may be produced and used as disclosed in the art or described herein, e.g., in the examples. Generally, the constructs may comprise a leader sequence linked to a single chain variable fragment (scFv), Fab or other antibody moiety, generally with a hinge or other linker between the scFv and a transmembrane domain (66). The transmembrane domain will be generally attached to an intracellular signaling domain and/or one or more co-stimulatory and/or co-inhibitory domains. Such domains can include CD28, CD3ζ, DAP12, DAP10, 2B4, CD137 (4-1BB), DNAM-1, γ chain of IgG Fc receptor and/or PD-1, CTLA-4 (67). Other co-stimulatory and co-inhibitory receptors have been reported in T and NK cells and intracellular domains of these receptors may be utilized to achieve desired response from the CAR-NK cells (see, e.g., FIG. 19). Activating and inhibitory receptors specific to NK cells include CD16 (FcγRIIIa), NKp30, NKp44, NKp46, NKp80, NKG2D, DNAM-1,TIGIT, CD94, NKG2C, NKG2A, 2B4, CD48, CD44, CD2, activating and inhibitory Killer-cell Ig like-receptors (KIR) (68). T cell specific co-stimulatory and co-inhibitory receptors include CD28, ICOS, CTAL-4, PD1, TIM1, TIM2, TIM3, TIM4, TIGIT, CD2, 2B4, DNAM-1, CD96, CD160, LAG3 and several others (69). Signaling domains from these and other co-stimulatory and/or co-inhibitory receptors may be incorporated in the subject CAR-NK therapy.

The majority of CAR constructs have utilized one or more scFv moieties to recognize one or more of specific antigens on target cells. Alternative antigen binding domains have been reported in the recent novel CAR constructs including antibody Fab domain, nanobodies (smaller, naturally occurring single-domain antibodies comprising the variable domains of antibody heavy chains (VH)), universal CARs based on avidin-streptavidin or other binding partners, receptors (e.g., NKG2D) and ligands (e.g. APRIL) (67). Any of these antigen-binding domains used for current CAR-T cells may be applied to the CAR-NK cells. Current forms of CAR-T and CAR-NK therapies target various antigens on target cells including but not limited to EGFR, EGFRvIII, CEA, Mesothelin, HER2 (Erbb2), MUC-1, MCD33, PSMA, GD2, EpCAM, Glypican-3, CD7, CD19, CD20, CD30, CD33, CD22, CD19/CD22, CD123, BCMA (CD269), ROBO1, NKG2DL, and CD138 (see, e.g., Pan, K., Farrukh, H., Chittepu, V.C.S.R. et al. CAR race to cancer immunotherapy: from CAR T, CAR NK to CAR macrophage therapy. J Exp Clin Cancer Res 41, 119 (2022)). Beyond cancer, viral antigens including HIV, HBV, HCV, CMV viral proteins have been targeted by scFv on CAR-T cells for infectious diseases (70). Other recent examples include fibroblast activation protein (FAP) for Fibrosis and auto-antigen expressing chimeric auto-antibody receptor (CAAR) T cells for auto-immune disease (71). All such target antigens may be selected to generate CAR NK cells expressing target antigen recognizing component (e.g., scFv/Fab/nanobody/receptor/ligand), e.g., as shown in FIG. 19.

To construct a CAR, an expression vector with specific CAR sequences in the desired order can be utilized. Various expression vectors are known and any such vector may be utilized to generate a complete CAR sequence (63). Multiple approaches have been utilized to deliver the CAR sequences in NK cells. Main approaches include viral transduction using lentiviruses or retroviruses, transfection with naked plasmid DNA, transposon mediated DNA integration or mRNA delivery by electroporation (86). Among these approaches, viral transduction is the most common choice to generate CAR-NK cells that are currently in pre-clinical and clinical development and fewer CAR-NK cell therapies have been reported with electroporation or nanoparticle-based transfection to deliver CAR constructs (63). Viral and non-viral based approaches mainly differ for their ability to induce stable CAR expression. Viral transduction is preferred for more stable and long-term (multiple weeks) expression of CAR in primary NK cells while electroporation results in transient expression of CAR lasting for about one week (63). To improve viral transduction of NK cells, several different transduction enhancers including statins, cationic polymers such as polybrene, protamine sulfate, dextran and retronectin have been used to generate CAR-NK cells using viral transduction and a choice of this depends upon the suitability based on the source of the NK cells (e.g., primary NK cells versus NK cell lines) (63). Further, cytokines and K562-mIL21/4-1BBL feeder cells are used to enhance the viral transduction of NK cells (63). CAR expression levels in primary NK cells greatly vary upon CAR expression (20-70%) while when using NK cell lines as source of CAR-NK cells, CAR expressing cells can be sorted by fluorescence activated cell sorting to achieve near 100% pure CAR-NK cell population (63).

### Modification of ECM Receptors on NK Cells

ECM proteins bind to the receptors on NK cells to modulate their functions and modifying the ECM receptor expression on NK cells is a promising therapeutic approach in various health conditions. The present methods include modification of one or more ECM receptors on NK cells and/or CAR-NK cells generated as disclosed above. Modification of one or more ECM receptors involves upregulation, downregulation, and/or knocking out of specific ECM receptor(s) via inhibitory nucleic acids (e.g., RNA interference) and/or genome editing techniques.

### Increased Expression of ECM Receptors

Generally speaking, the NK cells can be modified to overexpress an ECM receptor by transduction with a nucleic acid, e.g., expression vectors, containing a nucleic acid encoding an ECM receptor polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors can provide effective delivery of genes into cells. Whereas the transgene within a retroviral vector is typically stably integrated into the chromosomal DNA of the host, the transgene of an AAV vector usually exists as extrachromosomal episomes within the cytoplasm of infected cells. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include TCrip, TCre, T2 and TAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

Typically, an expression vector includes the nucleic acid in a form suitable for expression of the human an ECM receptor in an NK cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the level of expression of protein desired and whether regulated or inducible expression is desired. The expression vectors can be introduced into NK cells. The expression vector is preferably a vector suitable for expression in mammalian cells, and the expression vector's control functions can be provided by viral regulatory elements. For example, commonly used promoters are derived from polyoma, Adenovirus 2, cytomegalovirus and Simian Virus 40. See, e.g., Wang et al., Exp Hematol. 2008 July; 36(7):823-31.

In another aspect the invention provides NK cells that overexpress an ECM receptor nucleic acid molecule described herein, e.g., an ECM receptor nucleic acid molecule within a recombinant expression vector or an ECM receptor nucleic acid molecule containing sequences which allow it to homologously recombine into a specific site of the NK cell's genome. The term NK cell refers not only to the particular subject cell that is transduced but to the progeny or potential progeny of such a cell that contain the ECM receptor nucleic acid. Because certain modifications may occur in succeeding generations due to either mutation or environmental influences, such progeny may not, in fact, be identical to the parent cell, but are still included within the scope of the term as used herein.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

In another aspect, the invention features an NK cell or purified preparation of NK cells that include an ECM receptor transgene, which over-express an ECM receptor or express an ECM receptor in response to a stimulus.

Also provided herein are human NK cells, in which an endogenous ECM receptor is under the control of an exogenous regulatory sequence that does not normally control the expression of the endogenous ECM receptor gene, and that express the ECM receptor under circumstances in which a cell that lacks the exogenous regulatory sequence do not express the ECM receptor. The expression characteristics of an endogenous ECM receptor gene within a cell can be modified by inserting a heterologous DNA regulatory element into the genome of the cell such that the inserted regulatory element is operably linked to the endogenous ECM receptor gene. For example, an endogenous ECM receptor gene that is “transcriptionally silent,” e.g., not normally expressed, or expressed only at very low levels, may be activated by inserting a regulatory element capable of promoting the expression of a normally expressed gene product in that cell. Techniques such as targeted homologous recombination can be used to insert the heterologous DNA as described in, e.g., Chappel, U.S. Pat. No. 5,272,071; WO 91/06667, published in May 16, 1991.

The methods can also include identifying, selecting, and/or purifying those cells that overexpress ECM receptor, or that express ECM receptor over a desired level. The ECM receptor-expressing cells can be used for administration to a subject, can be frozen or otherwise stored for later administration to a subject.

### ECM Receptor Knockdown Knockout

Genome editing using the clustered regularly interspaced short palindromic repeats (CRISPR)/CRISPR-associated protein (Cas)-based gene editing in NK cells remains the most preferred choice of method to knock-out a particular gene in NK cells (72, 73). CRISPR-Cas9 system takes advantage of RNA-guided DNA recognition to specify the genomic modification locus to enable Cas9 mediated DNA cut precisely. Genome editing occurs through non-homologous end joining (NHEJ) and homology directed repair (HDR) mechanisms that takeover after double strand break (DSB) induction (73). Further modifications to improve the CRISPR/Cas9 system include introduction of inactivating mutation in one or both of the nuclease domains of Cas9 resulting in nickase activity of Cas9 or deactivated Cas9 that can be further fused with other enzymes with more specific base editing capabilities to enable more precise gene knockout with lesser off-target effects (73). Alternative approaches to knockout a specific gene involves utilizing zinc-finger nucleases (ZFN) and transcription activator-like nucleases (TALENs)-based systems. In contrast to CRISPR/Cas9 method, ZFN- and TALEN-based techniques target specific genomic loci via protein-DNA interactions (72). Any of the CRISPR-Cas9-, ZFN- and/or TALEN-based systems described herein or known in the art can be utilized to knockout one or more ECM receptors in NK cell therapeutic products. Such methods can be used to introduce deletions or stop codons or other alterations that prevent expression of a functional ECM receptor.

Inhibitory nucleic acids, e.g., antisense oligonucleotides, LNAs, RNA interference (RNAi), or shRNA, targeted to sequences encoding the ECM receptor can also be used. Unlike aforementioned techniques that are based on the modification of the genomic content within the nucleus, siRNA technology acts directly in the cytoplasm of cells and does not require nuclear import. Delivery of double-stranded short interfering RNAs (siRNA) of 21-23 nucleotides matching the specific sequences in the target gene mRNA results in short-term knockdown or downregulation of the gene expression (74). Alternatively, short hairpin RNA (shRNA) can be expressed in NK cells and processed by the Dicer endonuclease complex to achieve long-term expression of double-stranded siRNAs for more stable knockdown or downregulation of desired gene (74). RNAi techniques can be applied to target one or more ECM receptors in NK cells to achieve their knockdown.

Further modifications of Cas9 have been developed recently to expand the use of CRISPR/Cas9 system for gene activation or repression via epigenetic modifications. Dead Cas9 (dCas9) with defective nucleolytic activity but unaltered DNA-binding at the precise locus guided by RNA can be used to deliver effector molecule(s) with desired functions (73). The most common molecules that can be fused to dCas9 are transcription activators and repressors and/or epigenetic remodeling factors. Any of these factors can be used with the dCas9 based CRISPR-based activation (CRISPRa) or CRISPR-based interference (CRISPRi) to enable epigenetic regulation of one or more ECM receptors in NK cells (73).

Delivery of above-mentioned components in NK cells can be achieved by viral- or non-viral based transfection methods that include viral transductions using retroviral or lentiviral vectors, lipofection and/or electroporation to deliver DNA and/or RNA components to enable RNAi, CRISPR-based techniques or ectopic expression of transgenes. Similar to CAR expression in NK cells, one or more ECM receptors can be ectopically expressed in NK cells via viral transduction methods using retroviral and/or lentiviral vectors for stable integration of ECM receptor(s) gene and long-term expression of desired ECM receptor(s). Low transfection efficiency of NK cells is solved by delivering guide RNAs and Cas9 in the form of mRNA or protein after the formation of ribonucleoprotein (RNP) complexes via electroporation (75).

### ECM Receptors

Direct regulation of NK cell effector function occurs via receptor stimulation by ECM proteins. Lair1 is an inhibitory receptor with broad specificity for collagen and is expressed on many immune cells including human NK cells derived from various sources and selected NK cell lines (76, 77). With an example of Lair1 knockout in NK cell line NK92MI, our invention can be applied to modification of one or more of many such ECM receptor(s) on NK cells (Table 1). Integrins represent the principal ECM receptor family and function as transmembrane heterodimers comprised of Integrin α-(n=18) and β-subunits (n=8) with 24 different combinations (78, 79). Many of these combinations are expressed on NK cells (e.g., Integrin α2 with integrin-β1) and can be modified, e.g., via methods such as genetic and/or epigenetic manipulation or inhibitory nucleic acid techniques known in the art and described herein. Apart from integrin, there are several other non-integrin-based receptors like GPR56, CD44 and Toll-like receptors (TLR4, TLR2) that are expressed on NK cells and bind to different ECM components (80-82). Any of these or other ECM receptors expressed on NK cells can be modified through genetic and/or epigenetic manipulation techniques. Further, NK cells can be modified to induce or upregulate the expression of one or more of ECM receptors via any known methods including genetic and/or epigenetic manipulation techniques.

TABLE 1
ECM Proteins and Human Genomic Sequences

| ECM receptor (protein name) | GenBank human gene locus GRCh38.p14 |
|---|---|
| (LAIR1) leukocyte associated | NC_000019.10 (54351384 . . . 54376088, |
| immunoglobulin like receptor 1 | complement) |
| GPR56 aka (ADGRG1) adhesion G | NC_000016.10 (57619738 . . . 57665567) |
| protein-coupled receptor G1 | |
| (ITGA1) integrin subunit alpha 1 | NC_000005.10 (52787916 . . . 52959209) |
| (ITGA2) integrin subunit alpha 2 | NC_000005.10 (52989352 . . . 53094779) |
| (ITGA3) integrin subunit alpha 3 | NC_000017.11 (50056110 . . . 50090481) |
| (ITGA4) integrin subunit alpha 4 | NC_000002.12 (181457205 . . . 181538940) |
| (ITGA6) integrin subunit alpha 6 | NC_000002.12 (172427336 . . . 172506459) |
| (ITGAM) integrin subunit alpha M | NC_000016.10 (31259975 . . . 31332877) |
| (ITGAV) integrin subunit alpha V | NC_000002.12 (186590056 . . . 186680901) |
| ITGB1 integrin subunit beta 1 | NC_000010.11 (32900318 . . . 32958230, |
| | complement) |
| (ITGB2) integrin subunit beta 2 | NC_000021.9 (44885953 . . . 44928815, |
| | complement) |
| (ITGB3) integrin subunit beta 3 | NC_000017.11 (47253827 . . . 47313743) |
| (ITGB5) integrin subunit beta 5 | NC_000003.12 (124761948 . . . 124901418, |
| | complement) |
| Elastin binding protein (EBP) aka | NC_000003.12 (32961108 . . . 33097146, |
| (GLB1) galactosidase beta 1 | complement) |
| (NEU1) Neuraminidase 1 | NC_000006.12 (31857659 . . . 31862821, |
| | complement) |
| (CTSA) Cathepsin A | NC_000020.11 (45891335 . . . 45898820) |
| (LGALS3) Galectin 3 | NC_000014.9 (55129252 . . . 55145430) |
| (CD44) CD44 molecule (Indian | NC_000011.10 (35139171 . . . 35232402) |
| blood group) | |
| (TLR2) toll like receptor 2 | NC_000004.12 (153684280 . . . 153710637) |
| (TLR4) toll like receptor 4 | NC_000009.12 (117704403 . . . 117724735) |
| (DDR1) discoidin domain receptor | NC_000006.12 (30880970 . . . 30900156) |
| tyrosine kinase 1 | |
| (DDR2) discoidin domain receptor | NC_000001.11 (162630863 . . . 162787405) |
| tyrosine kinase 2 | |
| (PLAUR) plasminogen activator, | NC_000019.10 (43646095 . . . 43670169, |
| urokinase receptor | complement) |

### Methods of Treatment

Provided herein are methods of treatment that include administering the modified NK cells and compositions as described herein.

The methods include treatment of disorders associated with unwanted or abnormal immune activation. Generally, the methods include administering a therapeutically effective amount of a treatment comprising a composition comprising modified NK cells having increased expression of one or more EMC receptor proteins as described herein, to a subject who is in need of, or who has been determined to be in need of, such treatment. In some embodiments, the present methods can include identifying a subject who has a disorder associated with unwanted or abnormal immune activation associated with NK cell activation, and administering to the subject a therapeutically effective amount of a population of modified NK cells. Such disorders include autoimmune diseases, chronic inflammatory diseases, alloreactive immune response against transplanted grafts, and alloreactive immune response against a fetus for the treatment of pregnancy complications. In some embodiments, the disorder is a chronic inflammatory condition such as allergic asthma, atopic dermatitis, or inflammatory bowel disease (IBD). The autoimmune disease can be, for example, e.g. rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, or lymphocytic colitis. The transplant rejection can be a rejection of a kidney, lung, heart, liver, limb, skin, or multi-organ transplant. In cases of transplant rejection where T or B cells are involved, the methods can include administering a treatment comprising a composition comprising modified NK cells having reduced or eliminated expression of one or more EMC receptor proteins as described herein. Methods for identifying a subject who has transplant rejection associated with NK cell activation versus B or T cell activation are known in the art (see, e.g., Li XC. The significance of non-T-cell pathways in graft rejection: implications for transplant tolerance. Transplantation. 2010 Nov. 27; 90(10):1043-7).

The methods further include treatment of chronic infections, or disorders associated with abnormal apoptotic or differentiative processes. A chronic infection can last three weeks or more, or if the infection is recurrent despite completion of antibiotic treatment. Chronic infections can be viral or bacterial infections, e.g., a lung infection, pneumonia, septic shock, urinary tract infection, a gastrointestinal infection, an infection of the skin and soft tissue, an infection that modulates gut permeability, or an infection that modulates brain function, HIV infection, herpes infection, CMV infection, or any combination thereof. Abnormal proliferative disorders or cellular differentiative disorders, e.g., cancer, can include both solid tumors and hematopoietic cancers. In some embodiments, the disorder is a solid tumor, e.g., breast, prostate, pancreatic, brain, hepatic, lung, kidney, skin, or colon cancer. Generally, the methods include administering a therapeutically effective amount of a treatment comprising a composition comprising modified NK cells having reduced or eliminated expression of one or more EMC receptor proteins as described herein, to a subject who is in need of, or who has been determined to be in need of, such treatment. In some embodiments, the present methods can include identifying a subject who has cancer, and administering to the subject a therapeutically effective amount of a population of modified NK cells. The present methods can be used, e.g., in mammalian subjects, e.g., human or non-human veterinary subjects (e.g., non-human primate, mouse, rat, dog, cat, horse, or cow). In some embodiments, the modified NK cells are prepared from a population of the subject's own (autologous) NK cells; in some embodiments, the modified NK cells are prepared from cells obtained from a related or unrelated type-matched donor, or from a cell line or iPSC as known in the art or described herein. In some embodiments, the methods include administering a modified NK cell-based cancer immunotherapy, e.g., adoptive NK cell transfer, using modified natural killer cells (NK cells, i.e., CD3− cells), e.g., derived from healthy donor derived peripheral blood, induced pluripotent stem cells (iPSC), umbilical cord stem cells, oNKord cells (allogeneic partial HLA-matched NK cells derived from UCB-CD34+ progenitors), placenta-expanded NK cells (CYNK-001), CTV-1 lysate-primed human NK cells (CNDO-109-NK cells), or other natural sources; or modified NK-92 cells, NK-101 cells, or other NK cells obtained and expanded from patients with NK lymphomas, or variants of each of these, which can be further genetically modified to express chimeric antigen receptors (e.g., CAR-NK cells), Bi- and tri-specific killer engagers, BiKEs and TriKEs (e.g., CD16/IL-15/CD33 TriKE, GTB-3550), Tri-functional NK cell engagers (NKCEs) that crosslink both NKp46 and CD16; see, e.g., Liu et al., Journal of Hematology & Oncology 14, Article number: 7 (2021).

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the cancer is Skin cancer, Melanoma, squamous cell carcinoma, or breast cancer.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof.

Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, a composition described herein comprising modified NK cells is administered in combination with standard of care chemotherapy for a cycle or two or three.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity a composition as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

### EXAMPLES

The invention is further described in the following examples, which do not limit the scope of the invention described in the claims.

### Materials and Methods

The following materials and methods were used in the Examples below.

### Study Approval

Animal studies were approved by Massachusetts General Hospital Institutional Animal Care and Use Committee (IACUC).

### Mice

C57BL/6 albino WT, B2m^{-/-}, m157^{tg}, m157^{tg}, B2m^{-/-}, C57BL/6 Ncr1^{iCre}, ROSA^{mT-mG}, Ncr1^{iCre},Tgfbr2^{fl/fl}, Act-mOVA^{tg}, C57BL/6×BALB/c F1 Ncr1^{iCre}, ROSA^{mT-mG}, C57BL/6 Ncr1^{iCre}, ROSA^{DTR} C57BL/6×BALB/c F1 Ncr1^{iCre}, ROSA^{D}TR and C57BL/6×BALB/c F1 CD4^{Cre}, ROSA^{DTR} were bred in house. C57BL/6 mice (Charles River, Wilmington, MA, USA, strain code: 207) were used in the parabiosis, LEGENDplex and tumor experiments. C57BL/6-Ly5.1 mice (Charles River) were used for in vivo BM rejection experiments. C57BL/6 Hobit^{-/-} mice and Lair1^{-/-} mice were kind gifts from KP van Gisbergen, Sanquin Research and Landsteiner Laboratory, Amsterdam UMC, Amsterdam, The Netherlands, and Svetlana Komarova, Faculty of Dentistry, McGill University, Quebec, Canada, respectively. Mutant mice were genotyped using the primers listed in Table A. All mice were housed under specific pathogen-free conditions, given water and food ad libitum, in the animal facility at Massachusetts General Hospital in accordance with animal care regulations. All mice were closely monitored by the authors, facility technicians and an independent veterinarian when necessary. All procedures were performed according to the protocols approved by the Institutional Animal Care and Use Committee (IACUC) at Massachusetts General Hospital.

TABLE A
PCR primers:

| Gene | | SEQ ID |
|---|---|---|
| Name | Primer sequence | NO: |
| m157 | forward: TCAACTTCCGACGCAAAAGAAAT | 1. |
| | reverse: ACCGTCGATTCGTCAGTAACG | 2. |
| B2m | forward wild type: | 3. |
| | ACGGTGCAGACTGAGCTCTGTTTTCATCTGTCTTC | |
| | reverse: CAGGCGTATGTATCAGTCTCAGTGGGGGTGAATTC | 4. |
| | forward mutant: | 5. |
| | TCAGGATGATCTGGACGAAGAGCATCAGGG | |
| | reverse: CAGGCGTATGTATCAGTCTCAGTGGGGGTGAATTC | 6. |
| Act- | forward: CTTCCAGGATTCGGAGACAG | 7. |
| mOVA | reverse: TCTGAAAGGCATTGCTTGTG | 8. |
| Tgfbr2 | forward: TATGGACTG GCTGCT TTTGTA TTC | 9. |
| | reverse: TGGGGATAGAGGTAGAAAGACATA | 10. |
| Hobit | forward: TGCCTCCTTTTGACCCATGACATCCAAATT | 11. |
| | reverse: CTCTTTCTGGCTCATTTCCCATCTCCTCTC | 12. |
| | mutant: ATCAGTTCGCTTCTCGCTTC | 13. |
| Lair1 | forward wild type: GGTTTGAGGACAGACAGATGACC | 14. |
| | reverse: TTTATGAGGAGGACGACTGAGC | 15. |
| | forward mutant: CCAGGTGCACATGATCAGC | 16. |
| | reverse: TTTATGAGGAGGACGACTGAGC | 17. |

### Skin Transplantation

C57BL/6 albino ear skin from WT, B2m^{-/-}, m157^{tg}, m157^{tg}, B2m^{-/-} and Act-mOVA^{tg} mice was harvested after euthanasia, cartilage was removed and skin placed in a petri dish with 1×PBS (Life Technologies, Thermo Scientific, Grand Island, NY, USA, catalog no. 14190144). Recipient mice were anesthetized for the procedure with an intraperitoneal (i.p.) injection of 4.5 μL/g body weight of 10 mg/mL ketamine (KetaVed®, Boehringer, Ingelheim Vetmedica, Fort Dodge, IA, USA, catalog no. 045-290) combined with 0.5 mg/mL xylazine (AnaSed®, Akorn, Lake Fortes, IL, USA, catalog no. 139-236) sterile solution in addition to isofluorane (Baxter Healthcare Corporation, Deerfield, IL, USA, catalog no. 1001936040) as required. Mice were placed on a heating pad during and after the surgery. Backs of recipient mice were shaved and cleansed with 70% ethanol. A circular piece of recipient skin approximately 1.5 times the area of ear skin to be transplanted was excised from the back of recipient mice and subcutaneous fascia was removed. Donor ear skin was placed split side down and sutured using n° 6-0 silk surgical sutures (Ethicon, Puerto Rico, catalog no. K889H). Approximately one dozen equidistant sutures were made around the donor skin to attach the ear to the recipient tissue. Next, the skin transplant was coated with antibiotic ointment (Medline Industries, Northfield, IL, USA, catalog no. CUR001231) and protected with a 2 cm×2 cm patch of Vaseline gauze (Convidien, Mansfield, MA, USA, catalog no. 884413605). A bandage (Tensoplast®, BSN medical, Charlotte, NC, USA, catalog no. 02115-00) was wrapped around the torso of the mouse and sutured in place using n° 4-0 silk surgical sutures (Ethicon, Puerto Rico, catalog no. K871) on the dorsal region suturing into the back skin adjacent to the shoulders and hips. Seven days post-surgery the bandage was removed, and donor skin was monitored daily for any sign of inflammation and/or rejection. Pictures were taken every other day and ImageJ (Version 1.52a) was used to measure graft size. Recipient and donor mice were sex and aged-matched.

### Tissue Harvesting

Mice were anesthetized using ketamine/xylazine and 1 μg of monoclonal anti-CD45-BV605 (clone 30-F11, BioLegend, San Diego, CA, catalog no. 103155) was injected by retroorbital injection to label circulating CD45⁺ cells. After three minutes had elapsed, peripheral blood was collected by retroorbital bleeding and, following euthanasia, liver, spleen, lymph nodes, donor and recipient skin were collected for analysis. Red blood cells in peripheral blood, spleen and liver were lysed using red blood cell lysis buffer (RBC lysis buffer 10×, Biolegend, catalog no. 420301). After washing with 1×PBS/2% FCS/5 mM EDTA, 5×10⁶ cells were prepared for flow cytometry staining.

### Skin Harvesting

The back of the mouse was shaved and a large rectangle of shaved skin encompassing the donor graft and recipient skin was excised. Large regions of fat on the underside of the skin were carefully scraped away then the donor and recipient skin were separated with a razor blade. These were placed in separate dishes and chopped with scissors into −1 mm pieces. Each sample was then placed into a 15 mL tube with 10 mL digestion buffer (RPMI 1640 (Life Technologies, catalog no. 21870076), 200 U/mL Collagenase IV (Worthington Biochemical, Lakewood, NJ, USA)). Skin was incubated for 2 hours at 37° C. with shaking. Following incubation, digested skin was poured onto a 70 μM strainer placed in a 50 mL tube. The 15 mL tube was rinsed with −3 mL of 1×PBS/2% FCS/5 mM EDTA and added to the digested sample. Skin was mashed with a plunger through the 70 μM strainer which was then rinsed −3 times with a small volume of 1×PBS/2% FCS/5 mM EDTA. The cells were centrifuged at 300 g for 5 minutes at 4° C. and resuspended in 2.4G2 blocking solution before flow cytometry staining. NK cells were stained for activating, inhibitory receptors and CD107a surface expression for baseline evaluation. For the ex vivo NK cell stimulation assay, donor skin graft derived cells were resuspended in RPMI/10% FBS/1% penicillin and streptomycin (P/S, Thermo Fisher, Waltham, MA, catalog no. 14140122)/20 mM Glutamine and were plated on control IgG (clone MOPC-173, Biolegend, catalog no. 400203) or aNK1.1 (clone PK136, Biolegend, catalog no. 108703) pre-coated plate (Nunc™ MaxiSorp™ plate, Biolegend, catalog no. 423501) or treated with 1×PMA/Ionomycin solution (Biolegend, catalog no. 423301). The cells were incubated at 37° C./5% CO2 for total 4 hours with last 3 hours in the presence of 1 μL of anti-CD107a-BV421 (1D4B, Biolegend, catalog no. 121618), 1× Brefeldin A (Biolegend, catalog no. 420601) and 1× Monensin (Biolegend, catalog no. 420701). Following the co-culture, cells were washed with 1×PBS/2% FCS/5 mM EDTA and the pellet was resuspended in 2.4G2 blocking solution before flow cytometry staining.

### Liver Harvesting

The peritoneal cavity was opened to expose the liver and 5-10 mL of 1×PBS was injected into the hepatic portal vein using a 27G needle to perfuse the liver. The gall bladder was then removed and all lobes of the liver collected and mashed through a 70 μm filter sitting in a 50 mL tube using a syringe plunger. The filter was rinsed with 1×PBS/2% FCS/5 mM EDTA 3-4 times during the mashing procedure. The cell suspension was centrifuged at 300 g for 5 minutes at 4° C. then supernatant aspirated and the cell pellet washed with 10 mL of 1×PBS/2% FCS/5 mM EDTA. Following centrifugation and aspiration of supernatant the pellet was re-suspended in 25 mL of isotonic Percoll (8.44 mL Percoll [Healthcare Biosciences, Uppsala, Sweden, catalog no. 17-0891-01]/0.47 mL 20×PBS/16.09 mL 1×PBS) at room temperature (RT). Liver cell suspensions were centrifuged at 693 g for 12 minutes at RT with no brake, the leukocyte pellet at the bottom of the tube was then washed with 10 mL of 1× PBS/2% FCS/5 mM EDTA and red blood cells lysed (RBC lysis buffer 10×, BioLegend, catalog no. 420301). Following washing with 1×PBS/2% FCS/5 mM EDTA the pellet was resuspended in 2.4G2 blocking solution before flow cytometry staining.

### Flow Cytometry

Single-cell suspensions from all samples were prepared by straining through a 70 μm filter. 2.4G2 supernatant treated cells were stained in 1×PBS/2% FCS/5 mM EDTA with the appropriate surface antibodies (Table B) for 30 minutes at 4° C., washed and analyzed by flow cytometry. For intracellular staining, cells were fixed and permeabilized using True-Nuclear Transcription Buffer set (Biolegend, catalog no. 424401) according to the manufacturer's protocol then incubated with appropriate antibodies (Table B) for 60 min at RT, washed and analyzed by flow cytometry. For in vitro signaling experiments, following the specified time points, cells were fixed with an equal volume of pre-warmed 1×aldehyde based fixation buffer (Biolegend, catalog no. 420801) for 20 min at RT. Fixed cells were permeabilized by drop-wise addition of chilled methanol-based solution True-Phos Perm Buffer (Biolegend, catalog no. 425401) while mixed on a vortex. Cells were stored in −20° C. and dark for 1-3 days before washing them two times with −3 mL of 1×PBS/2% FCS/5 mM EDTA. Cells were subsequently stained with appropriate antibodies against surface markers and specific phosphorylated signaling proteins (Table B) for 60 min at RT, washed and analyzed by flow cytometry. Cells were assayed on a BD LSRFortessa X-20 flow cytometer (BD Bioscience, Billerica, MA, USA) and data were analyzed using FlowJo software Version 10 (Tree Star, Ashlad, OR, USA). NK cells in WT non-reporting mice were identified as CD3⁻NK1.1⁺NKp46⁺.

### Bone Marrow Transplantation

Bone marrow cells from B6-Ly5.1 (CD45.1), B6 WT (CD45.2) and B6 B2m^{-/-}(CD45.2) were collected from the tibia and femur by flushing bones with RPMI 1640 into a petri dish under sterile conditions. Cells were then counted and resuspended in RPMI 1640. Ly5.1:WT (control) and Ly5.1:B2m^{-/-} (test) BM cells were mixed 1:1 and intravenously injected into sub-lethally irradiated (450 cGy) Ncr1^{icre}, Rosa^{mT-mG} recipients. Three days later, spleens of recipient mice were harvested and the ratio WT:Ly5.1 and WT:B2m^{-/-} cells was determined by flow cytometry. % rejection was calculated as follows:

$$1 \; 00 - ( [ \frac{B 2 {m}^{- / -} / B 2 {m}^{+ / +} output}{B 2 {m}^{- / -} / B 2 {m}^{+ / +} input} ] \times 100 )$$

### Adoptive NK Cell Transfer

m157^{tg}, B2m^{-/-} donor skin was transplanted onto the back of C57BL/6 WT mice following the above-mentioned skin transplant procedure. On day one post-transplant, six spleens from Ncr1^{iCre}ROSA^{mTmG} were harvested and mashed through a 70 μm filter. Spleens were resuspended in RBC lysis for two minutes and washed with 1×PBS/2% FCS/5 mM EDTA. NKp46-GFP⁺ROSA⁻ cells were sorted using a Sony FX500 cell sorter (Sony Biotechnology, San Jose, CA, USA). Sorted cells were centrifuged and 8.5×10⁵ NKp46-GFP⁺ cells resuspended in 200 μL of sterile RPMI 1640 were filtered through a 40 μm strainer followed by intravenous injection into recipient mice using a sterile 28G syringe (0.5 mL BD insulin syringe, Franklin Lakes, NJ, USA, catalog no. 329461). Negative controls consisted of mice injected with 200 μL RPMI 1640 alone. 20 days post-transplant, mice were euthanized and peripheral blood, spleen, liver, donor and recipient skin were collected for analysis.

### Parabiosis

Parabiont partners were co-housed in the same cage for a week before the surgery. Eight to twelve week old female C57BL/6 WT mice were surgically connected to Ncr1^{iCre}, ROSA^{mTmG} weight and age-matched partner female mice. Surgery was performed on a heating pad and animals anesthetized using isoflurane. Longitudinal skin incisions were made on the shaved sides of each animal. Knee and elbow joints from each animal were first sutured using n° 4-0 surgical suture. Following attachment of joints, the skin of the animals was connected using n° 6-0 sutures ending with a double surgical knot. To minimize pain, 0.1 mg of Carpofren (Rimadyl, Zoetis, Brazil, catalog no. 141-199) was injected I.P. each day for two days post-surgery, in addition to close monitoring every day for signs of pain or stress. On day 21 post-parabiosis, ear skin from m157^{tg}, B2m^{-/-} was transplanted onto the back of the C57BL/6 WT mouse of each pair. After 20 days, mice were euthanized and peripheral blood, spleen, liver, donor and recipient skin from the C57BL/6 WT parabiont were collected for analysis.

### NK Cell Sorting

At day 20 post m157^{tg}, B2m^{-/-} transplantation onto Ncr1^{iCre}, ROSA^{mTmG} B6 mice and day 10 post m157^{tg} transplantation onto Ncr1icre ROSA^{mTmG} F1 mice, NK cells from blood, spleen and donor skin (cNK and trNK) and recipient skin (trNK cells) were sorted for RNA-sequencing. First, mice were anesthetized using ketamine/xylazine and 1 μg of monoclonal anti-CD45-BV605 (clone 30-F11, BioLegend) was injected via the retroorbital route three minutes before tissue harvest to label circulating CD45⁺ cells. Tissues were harvested as described above and, following staining, NKp46⁺ROSA-CD45⁺CD49a⁻CD49b⁺ (cNK) from peripheral blood and spleen, CD3⁻NKp46⁻ROSA⁻CD45⁻CD49a⁻CD49b⁺ (cNK), CD3⁻NKp46⁺ROSA⁻CD45⁻CD49a⁺CD49b⁺ (dpNK), CD3⁻NKp46⁺ROSA-CD45⁻CD49a⁺CD49b⁻ (trNK) from donor skin and CD3⁻NKp46⁺ROSA⁻CD45⁻ CD49a⁺CD49b⁻ (trNK) cells from recipient skin were sorted using a BD FACSAria II (BD Bioscience, Billerica, MA, USA). Sorted NK cells were collected in 15 mL tubes containing 3 mL of RPMI 1640 medium supplemented with 10% Fetal Bovine Serum (FBS, Corning, Manassas, VA, USA) and 5% P/S.

### RNA Sequencing and Analysis

Sorted cells were centrifuged, media aspirated and resuspended in 5 μL of TCL buffer with 1% β-mercaptoethanol (BME, Fisher Scientific, catalog no. 21-985-023) and added to a 96-well Eppendorf twin-tec barcoded plate (Eppendorf, NY, USA). Plates were stored at −80° C. until sequencing as described (60). Modified SmartSeq2 cDNA and Illumina Nextera XT library construction and sequencing were conducted at the Broad Institute of MIT and Harvard using an Illumina NextSeq 500 System (Boston, MA, USA). All samples were quasi-mapped to GRCm38 (mm10) using Salmon with the “gcBias” and “seqBias” options and collapsed down to gene-level abundance estimates using the “EnsDb.Mmusculus.v79” annotation package. All downstream differential expression analysis was carried out using DESeq2. The results table was restricted to genes with a minimum of 10 total counts across the dataset, and mitochondrial, pseudo-, and ribosomal genes were removed. Original data are available at the NCBI Gene Expression Omnibus (GEO), accession numbers: GSE148600. Gene set enrichment analysis (GSEA) (broad.mit.edu/gsea/) was used to identify significantly up- or down-regulated pathways in CD49b⁺ cNK cells in donor skin in comparison with CD49⁺ cNK cells in circulation (blood and spleen). GSEA analysis was performed using MSigDB 50 hallmark gene sets (h.all) and the curated 6226 gene sets (c2.all) and pathways containing enriched genes between 15 and 1000 genes were considered.

### Cell Lines

B16-F10 wildtype (WT) and B2m^{-/-} cell lines were maintained in RPMI 1640 media supplemented with 10% FBS and 1% P/S and cultured at 37° C./5% CO2. WT Mouse Embryonic Fibroblasts (WT-MEF) and m157-expressing MEF (m157-MEF) were a gift from Wayne Yokoyama, Washington University, St Louis, MO, USA. MEFs were cultured in RPMI 1640 media, containing 10% FBS, 1% P/S/20 mM Glutamine (Life Technologies, catalog no 25030-081).

### Melanoma Experiments

All tumor experiments were performed in eight to twelve-week-old C57BL/6 female WT or Ncr1^{iCre}, ROSA^{mT-mG} mice. For the S.C. tumor model, 2.5×10⁵ B16-F10 WT or B2m^{-/-} cells were subcutaneously injected into the shaved flanks of mice. Tumor growth was measured every other day using a digital caliper starting from day seven. Tumor volume was calculated as follows:

$$volume ( {cm}^{3} ) = \frac{( length \times {width}^{2} )}{2} .$$

To inhibit collagen deposition in and around the tumor, mice were administered with 60 mg/kg of Losartan Potassium (Fisher Scientific, catalog no. L02325G) or 40 mg/kg of 3,4-dihydroxybenzoic acid (DHB) (Sigma-Aldrich, catalog no. 37580) in 100 μL of sterile 1×PBS while the control mice were administered with the equivalent volume of sterile 1×PBS. These drugs were injected I.P. every day for the duration of the study starting the day of tumor inoculation.

For the collagenase/hyaluronidase experiment, 2.5×10⁵ B16-F10 B2m^{-/-} cells were resuspended in 1× collagenase and hyaluronidase mix (Stemcell, catalog no. 07912) and were subcutaneously injected into the shaved flanks of mice. To deplete collagen within the tumor, mice were administered with 1× collagenase and hyaluronidase mix (Stemcell, catalog no. 07912) in 100 μL of sterile 1×PBS while control mice were administered with the equivalent volume of sterile 1×sPBS. These enzymes were injected via intratumoral (I.T.) route starting on the day of tumor inoculation (day 0), day 1, day 2, day 3, day 4 and then every other day (day 6, 8, 10, etc.) until the end of the study. Except for day 3 tumor harvest, mice were euthanized when a tumor reached 2 cm in diameter in accordance with IACUC protocols. For early time point tumor analysis, specific Ncr1^{iCre}, ROSA^{mTmG} mice from PBS, Losartan and DHB group were euthanized on day 3 post tumor inoculation, the implanted tumor harvested and fixed in pre-chilled 95% ethanol overnight at 4° C. before subjecting them to subsequent processing.

For metastatic melanoma studies, mice were intravenously injected via tail vein with 2×10⁵ B16-F10 WT or B2m^{-/-} cells. Mice were euthanized 14 days post-injection, lungs were then harvested, fixed in 4% Paraformaldehyde (PFA, Sigma Aldrich, catalog no. P6148) and collected for histological analysis. Blinded quantification of the metastatic lung foci was performed on macroscopic images and the size of metastatic foci was measured using ImageJ software.

### Tumor Harvesting

Terminal subcutaneous tumors were excised with the skin and weighed before cutting in half. One half was placed in appropriate fixative while the other half was separated from the skin and chopped with scissors into −1 mm pieces. These were transferred to a 15 mL tube with 10 mL digest buffer (RPMI 1640 (Life Technologies, catalog no. 21870076), 200 U/mL Collagenase IV (Worthington Biochemical, Lakewood, NJ, USA)) and incubated for 2 hours at 37° C. with shaking. Following incubation, digested tumor was poured onto a 7 μM strainer placed in a 50 mL tube. The 15 mL tube was rinsed with −3 mL of 1×PBS/2% FCS/5 mM EDTA and added to the digested sample. The tumor was mashed with a plunger through the 7 μM strainer which was then rinsed ˜3 times with a small volume of 1×PBS/2% FCS/5 mM EDTA. The tumor-infiltrating CD45⁺ immune cells were isolated by magnetic-based positive selection using CD45 microbeads (Miltenyi Biotec, catalog no. 130-052-301) according to the manufacturer's protocol. The stained cells were passed through the LS columns (Miltenyi Biotec, catalog no. 130-042-401) placed between magnets for isolation of immune cells. The isolated CD45⁺ cells were centrifuged at 300 g for 5 minutes at 4° C. and resuspended in 2.4G2 blocking solution before flow cytometry staining.

### Cytokine, Poly(I:C) and DT Treatment

For cytokine treatment, mice were injected with 100 ng/mouse of mIL-12 (Biolegend, catalog no. 577002), 2 pg/mouse of mIL-15 (Shenandoah Biotechnology, Warwick, PA, catalog no. 200-07) and 100 ng/mouse of mIL-18 (Shenandoah Biotechnology, catalog no. 200-83) in 200 μL of sterile 1×PBS. For the combination Poly(I.C) and cytokines, mice were injected with 200 pg/mouse of Poly(I.C) (Sigma-Aldrich, catalog no. P9582), 2 pg/mouse of mIL-15 (Shenandoah Biotechnology, catalog no. 200-07) and 100 ng/mouse of mIL-18 (Shenandoah Biotechnology, catalog no. 200-83) in 200 μL of sterile 1×PBS. Treatments were injected I.P. starting the day of the skin transplant (day 0), day 2 and day 4 and then injected S.C. at days 7, 10, 13, 16 and 19 post-transplant. Diphtheria toxin (DT, Sigma-Aldrich, catalog no. D0564) was injected I.P. at 500 ng/mouse the day before the transplant and 200 ng/mouse at days indicated in fig. S5E combined with NK or T cell depletion.

### Nk Cell Depletion, T Cell Depletion and Checkpoint Blockade

Mice were I.P. injected with 500 pg/mouse of IgG isotype control (Southern Biotech, Birmingham, AL, USA, catalog no. 0107-01) or depleting anti-NK1.1 antibody (clone PK136, BioXcell, West Lebanon, NH, USA, catalog no. BE0036) two days before tumor cell injection and 250 pg/mouse every other day starting the day of the tumor cell injection. For depletion of NK or T cells (anti-CD4 antibody, clone GK1.5, BioXcell; anti-CD8a antibody, clone YTS 169.4, BioXcell) in a skin transplant setting, 500 pg/mouse of each depleting antibody were injected the day of the transplant and 250 pg at days indicated in fig. S5E. Anti-mouse PD-1 (clone 29F.1A12, BioXCell, catalog no. BE2073), anti-TIGIT (clone 1G9, BioXCell, catalog no. BE0274) and anti-CTLA4 (clone 9D9, BioXCell, catalog no. BE0164) antibodies were I.P. injected at 200 pg each/mouse at day −2 then every three days beginning at day 0 of skin transplantation.

### In Vitro Experiments

Prior to the experiment, blood from C57BL/6 WT and m157^{tg} mice was collected, incubated overnight at 4° C., centrifuged and serum was isolated from the pellet. Spleens from C57BL/6 mice were harvested, mashed through a 70 μm filter and cells were resuspended in RBC lysis for two minutes. After washing with 1× PBS/2% FCS/5 mM EDTA, cells were counted and 5×10⁶ splenocytes resuspended in 500 μL RPMI 10% FBS/1% P/S/20 mM Glutamine and 10% serum of either WT or m157^{tg} mice then plated in a 24-well plate and incubated for 3 h at 37° C./5% CO2. Cells were then washed, re-plated and 200 μL of a mixture containing 1×10⁵ WT-MEF or m157-MEF, mIL-12 (10 ng/mL, Biolegend, catalog no. 577002) or mIL-12/mIL-18 (12.5 ng/mL, Shenandoah Biotechnology, catalog no. 200-83) was added to the well. After incubation for another hour at 37° C./5% CO2, 1 μL of anti-CD107a-eF660 (clone 1D4B, eBioscience, catalog no. 50-1071-82), 1× Brefeldin A (Biolegend, catalog no. 420601) and 1× Monensin (Biolegend, catalog no. 420701) were added and cells were incubated for 6 hours. Then, the supernatant was removed, cells were washed and resuspended in 2.4G2 blocking solution before flow cytometry staining. Cells were assayed on a BD FACSCanto (BD Biosciences).

For in vitro experiments in the presence of ECM components, Collagen I (Rat tail, Sigma, catalog no. 08-115), collagen III (Human placenta, Sigma, catalog no. C4407), collagen IV (Mouse, Sigma, catalog no. C0543), Elastin (Mouse lung, Sigma, catalog no. E6402), Laminin (Mouse, Thermo Fisher, catalog no. 23017015) and Fibronectin (Rat plasma, Sigma, catalog no. F0635) were diluted in 1×PBS and coated at 20 μg/cm² in a 24 well tissue culture plate for 30 minutes at RT with gentle shaking followed by overnight at 4° C. Spleens from Ncr1^{iCre}, ROSA^{mT-mG}, WT or Lair1^{-/-} mice were harvested as described above. Then, 5×10⁶ splenocytes resuspended in RPMI 10% FBS/1% P/S/20 mM Glutamine were mixed with 20 μg of respective ECM protein per well and were incubated at RT for 10 minutes. Splenocytes and ECM mix were plated onto the ECM-coated well and were incubated at 37° C./5% CO2 for 2 hours (short ECM exposure) or 17 hours (long ECM exposure). At the end of the incubation, 1×10⁵ MEF (WT or m157) cells were added to each well to obtain a 50:1 splenocyte:MEF ratio. For cell signaling experiments, hydrogen peroxide (H₂O₂) at a final concentration of 2.5 mM or 1×10⁵ MEF-m157 cells were added to each well except in “0 min” wells which were unstimulated and fixed immediately. Splenocytes were stimulated with 2.5 mM H₂O₂ for 2 min and 5 min or were co-cultured with MEF-m157 for 30 min and 60 min (signaling) or 7 hours (CD107a and IFN□) at 37° C./5% CO2. Splenocytes and MEF in co-culture were supplemented with mIL-12 (10 ng/mL, Biolegend, catalog no. 577002), mIL-15 (1 ng/mL, Shenandoah Biotechnology, catalog no. 200-07) and hIL-2 (Biolegend, catalog no. 589104; only long ECM exposure) as well as for CD107a/IFN□ experiments. 1 μL of anti-CD107a-BV421 (1D4B, Biolegend, catalog no. 121618), 1× Brefeldin A (Biolegend, catalog no. 420601) and 1× Monensin (Biolegend, catalog no. 420701) were added. Following the co-culture, cells were washed with 1×PBS/2% FCS/5 mM EDTA and the pellet was resuspended in 2.4G2 blocking solution before flow cytometry staining.

### NK Cell Enrichment

NK cells purified from spleens under sterile conditions were used for LEGENDplex experiments. Spleens from 8-9-week-old C57BL/6 female WT mice were harvested and mashed through a 70 μm filter. Cells were resuspended in RBC lysis for two minutes and washed with 1×PBS/2% FCS/5 mM EDTA. NK cells were enriched by negative selection using an NK cell isolation kit (Milteny Biotec, catalog no. 130-115-818) and magnetic LS columns (Milteny Biotec, catalog no. 130-042-401) according to the manufacturer's instructions. Enrichment of isolated NK cells was confirmed by flow cytometry and was 88-90% CD3-NK1.1⁺NKp46⁺ cells. Enriched NK cells were resuspended in RPMI 1640 media supplemented with 10% FBS and 1% P/S.

### LEGENDplex

ECM protein coating on wells was performed as previously described. 1×10⁵ enriched splenic NK cells was mixed with 20 μg of respective ECM protein per well in RPMI 1640/10% FBS/1% P/S/20 mM Glutamine and incubated at RT for 10 minutes. NK cells and ECM mix were plated onto the ECM-coated well and were incubated at 37° C./5% CO2 for 2 hours. At the end of the incubation, 1×10⁵ MEF (WT or m157) cells were added to the respective wells to obtain a 1:1 NK:MEF ratio. NK cells and MEF were co-cultured at 37° C. 5% CO2 for 24 hours supplemented with mIL-12 (10 ng/mL, Biolegend, catalog no. 577002) and mIL-15 (1 ng/mL, Shenandoah Biotechnology, catalog no. 200-07-100ug). The supernatant was collected at 12- and 24-hour intervals, centrifuged to remove cells/cell debris and subsequently frozen at −80° C. until further use. Quantification of murine cytokines and chemokines (CCL2, CCL5, CXCL9, CXCL10) in supernatants was performed with LEGENDplex kits (Biolegend, catalog nos. 740451, 740622) according to manufacturer's instructions. In brief, a mix of capture beads with distinct size and fluorescence was incubated with undiluted S/N or standard dilutions for 2 hours at RT with constant shaking. Beads were washed, a biotinylated detection antibody was added and the plate was incubated for 1 hour at RT with constant shaking. The streptavidin-PE conjugate was added and further incubated for 30 minutes at RT on a shaker. Beads were washed and subsequently acquired on a BD LSRFortessa X-20 flow cytometer. Analysis and quantification of the results were done using LEGENDplex data analysis software (BioLegend). Quantification was reported as pg/ml for CCL2, CCL5, CXCL10 and as MFI for CXCL9.

### Histology, Collagen, Elastin and Immunofluorescence Staining

Skin grafts were collected and incubated in 4% PFA/25% sucrose (Fisher Scientific, Hampton, NH, USA, catalog no. S5-3) solution at 4° C. for 16 hours. The next day, slices were equilibrated for 4h in 50% sucrose solution, embedded in OCT (Fisher Scientific, catalog no. 23-730-571), snap frozen and stored at −80° C. For paraffin embedding, lungs and tumor were collected and fixed in 4% PFA overnight at 4° C. Subsequently, tissues were dehydrated in ethanol, processed and embedded in paraffin according to standard histology processes. For paraffin embedding of Ncr1^{icre}, Rosa^{mT-mG} a different fixation protocol was used to maintain the NKp46-GFP and Tdt-Tomato-ROSA fluorescence. Tumors from Ncr1^{icre}, Rosa^{mT-mG} mice were collected and fixed in pre-chilled 95% ethanol overnight at 4° C. Tissues were subsequently dehydrated in 100% ethanol, cleared in 100% xylene and embedded in paraffin according to standard histology processes. For IF staining of frozen tissue, 7 μm sections of skin were cut on a cryostat, re-hydrated in three washes of 1× Tris-buffered-saline (TBS) for 2 minutes each followed by blocking of nonspecific protein with 5% bovine serum albumin (BSA, Fisher Scientific, catalog no. BP1600) and 5% goat serum (Sigma-Aldrich, catalog no. G9023) in 1×TBS. Sections were stained overnight at 4° C. with primary antibodies (Table B). The following day, slides were washed as above and incubated for two hours at RT with secondary antibodies conjugated to fluorochromes (Table B). After washing as above, sections were incubated with a 1:4000 dilution of DAPI (Invitrogen, Carlsbad, CA, catalog no. D3571) in 1×TBS for 5 minutes at RT, washed, air-dried and coverslips mounted with Prolong Gold Antifade Reagent (Invitrogen, catalog no. P36930). Five or six randomly selected fields of view at 200× total magnification were obtained for each section using a Zeiss Axio Scan (Zeiss, Oberkochen, Germany). Blinded manual counting of NKp46⁺, CD3⁺, CD4⁺ and CD8⁺ T cells were performed using ZEN Blue Software (Zeiss, Oberkochen, Germany). Automated counting was performed for CD11b, F4/80 and Arginasel stains in the whole tumor region of the scanned slides by Halo3.0 software (Indica Labs, Albuquerque, USA). For IF of paraffin-embedded tissues, 5 μm sections were rehydrated and permeabilized with 1×PBS supplemented with 0.2% Triton X-100 (Thermo Fisher Scientific, catalog no. BP151) for 5 minutes. Antigen retrieval was then performed using a Cuisinart pressure cooker for 20 minutes at high pressure in antigen unmasking solution (Vector Laboratories, Burligame, CA, catalog no. H-3300). Slides were then washed three times for three minutes each in 1×PBS supplemented with 0.1% Tween 20 (Sigma-Aldrich, catalog no. P1379). Sections were blocked, stained (Table B) and mounted as described above. Elastin fiber staining was performed using Verhoeff Van Gienson Elastin Stain kit (ab150667, Abcam) following the manufacturer's protocol. For collagen staining, Masson's Trichrome staining (Polysciences, Warrington, PA, catalog no. 25088) was performed following the instructions of the manufacturer. Whole slide imaging was performed using a NanoZoomer S60 Digital slide scanner (Hamamatsu, Japan) or Axio Scan.Z1 (Zeiss, Germany) and analyzed with NDP-view2 software (Hamamatsu) or ZEN Blue Software (Zeiss, Oberkochen, Germany), respectively.

### B2M Analysis in Human Cancers

B2Mgene alterations and expression in solid cancer and leukemia samples in TCGA and other public databases were obtained and analyzed through cBioPortal for Cancer Genomics at cbioportal.org/

### Statistical Analysis

Graphs show mean values ±standard deviation (SD). The numbers of mice per group used in each experiment are annotated in the corresponding figure legend as n. Graphs and statistical analysis were performed using GraphPad Prism 8 (La Jolla, CA, USA) and RStudio. All tumor quantifications were performed blindly. Two-tailed Fisher's exact test was used to compare skin graft rejection grades and lymph node metastatic load among groups. Two-way ANOVA with Sidak's multiple comparison test was used to compare tumor growth over time between different groups. Comparisons of survival were performed with the Log-rank test. Two-tailed Mann-Whitney U test was used for all the other comparisons. A P value of less than 0.05 was considered significant.

TABLE B
antibodies for tissue analysis

| Immunostaining | | |
|---|---|---|
| Antibodies | Clone | Company |
| Rat Anti-Mouse CD3 | CD3-12 | Abcam, Cambridge, MA |
| Rabbit Anti-Mouse CD8 | D4W2Z | Cell Signaling Technology, Danvers, |
| | | MA |
| Rabbit Anti-Mouse CD4 | EPR19514 | Abcam |
| Rabbit Anti-mouse | EPR3776 | Abcam |
| vimentin | | |
| Rabbit Anti-Mouse | EPR19387 | Abcam |
| CD11b | | |
| Rabbit Anti-Mouse F4/80 | D2S9R | Cell Signaling Technology |
| Rabbit Anti-Mouse | D4E3M | Cell Signaling Technology |
| Arginase1 | | |

Secondary Antibodies and staining kits

| | |
|---|---|
| Goat anti-Rat IgG, Alexa Fluor ® 647 conjugate | Thermo Fisher Scientific, Waltham, |
| | MA |
| Goat anti-Rabbit IgG, Alexa Fluor ® 568 conjugate | Thermo Fisher Scientific |
| Goat anti-Rabbit IgG, Alexa Fluor ® 488 conjugate | Thermo Fisher Scientific |
| Elastic stain Kit - Verhoeff Van Gienson (EVG) | Abcam |
| Stain | |
| Masson's Trichrome Stain Kit | Polysciences, Warrington, PA |

Flow Cytometry Antibodies

| | | |
|---|---|---|
| Mouse | | |
| CD3-eF450 | 145-2C11 | BioLegend |
| CD3-AF700 | 145-2C11 | BioLegend |
| CD3-PerCP-Cy5.5 | 145-2C11 | BioLegend |
| CD3-PE | 145-2C11 | BioLegend |
| CD3-PE-Cy7 | 145-2C11 | BioLegend |
| CD3-APC-eF780 | 145-2C11 | eBioscience |
| CD3-BUV395 | 17A2 | BD-Biosciences |
| CD4-eF450 | GK1.5 | BioLegend |
| CD4-PerCP-Cy5.5 | GK1.5 | BioLegend |
| CD4-AF700 | RM4-5 | BioLegend |
| CD8-AF700 | 53-6.7 | BioLegend |
| CD8-APC-Cy7 | 53-6.7 | BioLegend |
| CD8-BUV395 | 53-6.7 | BD-Biosciences |
| CD45-BV605 | 30-F11 | BioLegend |
| CD45-PerCP-Cy5.5 | 30-F11 | BioLegend |
| CD45.1-APC-eF780 | A20 | eBioscience |
| CD45.2-eF450 | 104 | eBioscience |
| CD19-PE-Cy7 | 1D3 | eBioscience |
| CD19-APC-Cy7 | 6D5 | BioLegend |
| CD49a-PerCP-Cy5.5 | Ha31/8 | BioLegend |
| CD49b-APC | DX5 | BioLegend |
| CD49b-APC-Cy7 | DX5 | BioLegend |
| NK1.1-PE | PK136 | BioLegend |
| NK1.1-PECy7 | PK136 | BioLegend |
| NK1.1-APC | PK136 | BioLegend |
| NK1.1-PerCP-Cy5.5 | PK136 | eBioscience |
| NKp46-FITC | 29A1.4 | BioLegend |
| NKp46-PE | 29A1.4 | BioLegend |
| NKp46-BV610 | 29A1.4 | BioLegend |
| NKG2D-APC | CX5 | eBioscience |
| NKG2A-APC | 16A11 | BioLegend |
| CD11b-BV395 | M1/70 | eBioscience |
| CD48-AF700 | HM48-1 | BioLegend |
| CD69-eF450 | H1.2F3 | BioLegend |
| CD69-AF647 | H1.2F3 | BioLegend |
| DNAM1-PE-Cy7 | 10E5 | BioLegend |
| FoxP3-AF700 | MF-14 | BioLegend |
| Ly49H-AF647 | 3D10 | BioLegend |
| Ly49H-PE | 3D10 | BioLegend |
| Ly49H-PE-Cy7 | 3D10 | BioLegend |
| Ly49C/I-BV605 | 5E6 | BD-Biosciences |
| Ly49A-Pacific Blue | YE1/48.10.6 | BioLegend |
| CD103-AF647 | 2.00E+07 | BioLegend |
| CD107-eF660 | 1D4B | eBioscience |
| CD107a-BV421 | 1D4B | BioLegend |
| TRAIL-PE | N2B2 | BioLegend |
| TGFBRII-APC | Polyclonal | R&D systems, Minneapolis, MN |
| TIGIT-AF700 | 2190A | R&D systems |
| IL4rα-PE-Cy7 | I015F8 | Biolegend |
| Ccr7-PE-Cy7 | 4B12 | Biolegend |
| IFNγ-eF450 | XMG1.2 | eBioscience |
| IFNγ-AF700 | XMG1.2 | BioLegend |
| IFNγ-PE-Cy7 | XMG1.2 | eBioscience |
| IFNγ-BV605 | XMG1.2 | BioLegend |
| TNFα-BV605 | MP6-XT22 | BioLegend |
| Granzyme B-AF647 | GB11 | BioLegend |
| Granzyme B-Pacific Blue | GB11 | BioLegend |
| CD279 (PD-1)-BV605 | 29A.1A12 | BioLegend |
| Phospho-PLCg1 (Tyr783)-AF647 | D6M9S | Cell Signaling Technology, Danvers, |
| | | MA |
| Phospho-PLCg1 (Tyr783)-PE | A17025A | BioLegend |
| Phospho-ERK1/2 | 6B86B9 | BioLegend |
| (Thr202/Tyr204)-BV421 | | |
| Phospho-NFKB p65 (Ser536)- | 93H1 | Cell Signaling Technology, Danvers, |
| AF647 | | MA |
| Phospho-STAT3 (Tyr705)-BV421 | 13A3-1 | BioLegend |
| Phosho-STAT5 (Tyr695)-PerCP- | SRBCZX | Thermo Fisher Scientific |
| eFluor710 | | |

### Example 1. NK Cell-Activating Ligand Together with Loss of MHC-I is Insufficient to Induce Rejection of Syngeneic Skin Graft

In order to assess the requirements for NiK cell cytotoxicity in solid organs, we developed a syngeneic skin transplant model. Ear skin from donor mice on an albino B6 background was transplanted onto the back of B6 recipients. WT and m157^{tg} mice with or without the deletion of beta-2 microglobulin (B2m) gene were used as donors. Ncr1^{iCre}, ROSA^{mT-mG} reporter mice expressing green fluorescent protein (GFP) in NKp46⁺NIK cells and TdTomato in all other cells were used as recipients (FIG. 1A) (26). This approach enabled us to examine the impact of an NIK cell-specific activating ligand, loss of MHC-I (through B2m deletion) or the combination of both on skin transplant outcomes while precisely distinguishing donor from recipient skin by skin graft color (albino ear skin) and histologically by immunofluorescence (IF) imaging. WT, B2m^{-/-}, m157^{tg}, and m157^{tg}, B2m^{-/-} skin grafts were monitored for signs of inflammation and rejection over time (FIG. 10). At 20 days post-transplant, B2m^{-/-} skin grafts showed no signs of rejection (FIGS. 1, B and C). In contrast, B2m^{-/-} BM transplants were readily rejected in WT B6 recipients. m157^{tg} and m157^{tg}, B2m^{-/-} skin grafts displayed minor signs of inflammation, which was most apparent in the latter (FIGS. 1, B and C), yet no significant change in the size of the skin grafts was observed between the four groups from day 10 to day 20 post-transplant (FIG. 1D). Monitoring m157^{tg}, B2m^{-/-} skin grafts out to day 60 post-transplant revealed no additional changes. Massive numbers of NKp46-GFP⁺ NK, CD4⁺T and CD8⁺ T cells infiltrated the skin and the epidermis of m157^{tg} and m157^{tg}, B2m^{-/-} compared with WT grafts (FIGS. 1, E and F). B2m^{-/-} skin grafts also showed higher NK and T cell infiltrates compared with WT grafts (FIGS. 1, E and F). No NK or T cell infiltrate was detected in the recipient skin adjacent to the skin grafts across the four groups. These findings establish that the expression of NK cell-specific activating ligand, m157, in the skin grafts results in markedly enhanced recruitment of NK cells and an associated increase in T cell numbers in the grafts but does not lead to graft rejection.

Next, we examined the strength of m157-Ly49H interaction in NK cells that infiltrated the skin grafts. Recipient-derived CD49b⁺ cNK and CD49a⁺ trNK cells were found in m157-expressing skin grafts, which expressed Ly49H and exhibited m157-induced Ly49H downregulation compared with Ly49H⁻ and Ly49H⁺ cNK and Ly49H⁻ trNK cells in the recipient's liver (27). No significant alteration in NKG2D, CD48, DNAM1, NKG2A, Ly49A and Ly49C/I expression on the surface of CD49b⁺ cNK and CD49b⁺CD49a⁺ double-positive NK (dpNK) cells in m157^{tg}, B2m^{-/-} skin graft was detected. Although a large number of NKp46-GFP⁺ cNK and trNK cells infiltrated m157-expressing skin grafts, these cells did not upregulate IFNγ, TNFα or granzyme B expression. NK cells isolated from blood, spleen and lymph nodes of WT recipients transplanted with m157^{tg} skin did not downregulate Ly49H expression in contrast to those of m157^{tg} mice, ruling out systemic NK cell hyporesponsiveness in recipient animals (27). In addition, WT NK cells were fully responsive to m157 stimulation in the presence of serum from m157^{tg} mice in vitro, ruling out the possibility that a soluble form of m157 blocked NK cell activation in m157-expressing skin grafts. NK cells can become hyporesponsive upon chronic exposure to the loss of MHC-I (28, 29). To investigate this concept in our transplant system, we tested the ability of m157^{tg}, B2m^{-/-} skin graft-infiltrating NK cells at day 10 post transplantation to degranulate and produce IFNγ in response to ex vivo stimulation. Compared with splenic cNK cells, skin graft-derived cNK cells had comparable expression levels of MHC-I specific inhibitory receptors and were not found to be degranulating at baseline. However, upon ex vivo co-culture, Ly49H⁺ cNK cells from m157^{tg}, B2m^{-/-} skin graft showed robust degranulation and IFNγ secretion compared with splenic Ly49H⁺ cNK cells in control IgG-coated, αNK1.1 antibody-coated plates as well as upon phorbol myristate acetate plus ionomycin (PMA/Ion) stimulation. We did not observe any increase in degranulation and IFNγ secretion by Ly49H⁻ cNK cells in the skin graft versus spleen except for a minor increase in degranulation of skin Ly49H⁻ NK cells in IgG-coated plate. These findings demonstrate that Ly49H⁺ cNK cells from m157^{tg}, B2m^{-/-} skin graft are not hyporesponsive and are fully capable of activation and degranulation outside of the skin.

To investigate the timing of inflammation in m157-expressing skin grafts, we compared m157^{tg} grafts to skin grafts expressing a model T cell antigen, Ovalbumin (OVA). WT B6 mice were transplanted with m157^{tg}, m157^{tg}, B2m^{-/-}, mOVA^{tg} and mOVA^{tg}, B2m^{-/-} skin grafts and monitored. As expected, mOVA^{tg} donor skin grafts were mostly rejected by day 20 post-transplant while the absence of MHC-I inhibited the rejection of mOVA^{tg}, B2m^{-/-} grafts (FIG. 1G). The paradoxical increase in inflammation in m157^{tg}, B2m^{-/-} compared with m157^{tg} grafts supported the dominant role of NK rather than T cells in initiating the immune response in m157-expressing grafts. However, we noticed that inflammation in m157^{tg}, B2m^{-/-} skin grafts developed after day 12 post-transplant, along a similar adaptive immune response timeline to mOVA^{tg} grafts (FIG. 1G). These data suggest that early NK cell activation following m157^{tg} skin transplantation may initiate a delayed T cell response leading to skin inflammation. Nonetheless, NK cell activation against syngeneic skin expressing m157 and lacking MHC-I is insufficient to cause skin graft rejection.

### Example 2. CNK Cells Recruited from the Circulation into Skin Grafts Give Rise to trNK Cells and Fail to Reject the Graft Upon Additional Cytokine Stimulation

To overcome the apparent inhibition of cytotoxicity experienced by NK cells in m157-expressing skin grafts, we treated the recipient mice with IL-15, IL-18 and a TLR3 agonist, poly(I:C), or IL-12, IL-15 and IL-18. These NK cell survival and activating factors are commonly used to enhance NK cell cytotoxicity (30, 31). However, m157^{tg} and m157^{tg}, B2m^{-/-} skin grafts treated with these agents showed no sign of rejection at day 21 post-transplant (FIGS. 2, A and B). Although, combination of IL-12, IL-15 and IL-18 had no effect on NK cell recruitment and their activation and maturation profile, these cytokines appeared to enhance the conversion of cNK cells into trNK cells in the skin graft, which may be mediated by IL-15 (32, 33). To elucidate the basis for this profound lack of NK cell cytotoxicity, we investigated the phenotype of CD49b⁺ cNK and CD49a⁺ trNK cells, which were highly enriched in m157^{tg}, B2m^{-/-} skin grafts (FIG. 2C). Recipient liver cNK cells were largely negative for CD69, CD103 and TRAIL, while the large majority of liver trNK cells expressed CD69 and TRAIL. cNK cells in m157^{tg}, B2m^{-/-} skin grafts expressed CD69 with minimal CD103 and TRAIL expression. In contrast, two-thirds of trNK cells in the skin graft co-expressed CD69 and CD103 supporting their tissue-resident status. Surprisingly, trNK cells in the skin graft expressed low levels of TRAIL. Combining the ratio of NK cell subtypes from flow cytometry data with absolute NK cell counts in histological sections showed that CD49b⁺ cNK cells were significantly increased in B2m^{-/-}, m57^{tg} and m157^{tg}, B2m^{-/-} skin grafts compared with WT grafts (FIG. 2D). In addition, CD49a⁺ trNK cells and CD49b⁺CD49a⁺ dpNK cells were markedly increased in m157^{tg} and m157^{tg}, B2m^{-/-} skin grafts compared with WT grafts, which largely lacked trNK and dpNK cells (FIG. 2D). In contrast to cNK cells in WT skin grafts, all three NK cell subsets in m157^{tg}, B2m^{-/-} skin grafts expressed Ly49H at levels indicative of Ly49H downregulation upon binding to m157 on the donor skin cells (FIG. 2E). Labeling the circulating immune cells with intravenous (I.V.) injection of anti-CD45 antibody further demonstrated that the majority of cNK cells in WT and m157^{tg} skin grafts were CD45 negative and had exited circulation similar to trNK cells in the skin. Considering that trNK cells do not migrate (34-36) and ILCP-derived trNK cells are Ly49H negative (36), the large population of NKp46-GFP⁺ Ly49H+trNK cells in m157-expressing skin grafts indicates that circulating cNK cells give rise to trNK cells in the donor skin.

To determine whether NKp46-GFP⁺ NK cells in m157^{tg}, B2m^{-/-} skin grafts were recruited from circulation or the surrounding recipient skin, we performed a parabiosis experiment. Ncr1^{iCre}, ROSA^{mT-mG} mice were partnered with WT mice and their circulatory systems were allowed to conjoin for 20 days before m157^{tg}, B2m^{-/-}skin was transplanted onto the WT parabiont (FIG. 3A). At 20 days following skin transplantation, NKp46-GFP⁺ NK cells were found in the spleen and the skin graft, the majority of which were negative for anti-CD45 antibody injected I.V. (FIG. 3A). NKp46-GFP⁺ NK cells migrated into the dermis and epidermis of m157^{tg}, B2m^{-/-} skin followed by a large T cell infiltrate into the skin grafts (FIG. 3B). To directly test recruitment from circulation, we sorted splenic NKp46-GFP⁺ CD49b⁺CD49a⁻ cNK cells from Ncr1^{iCre}, ROSA^{mT-mG} mice and injected them I.V. into WT mice one day following m157^{tg}, B2m^{-/-} skin transplantation. After 20 days, NKp46-GFP⁺ NK cells were readily identified circulating in the recipient liver and a few NKp46-GFP⁺ NK cells were detected in the skin grafts, which had exited the circulation (FIG. 3C). Together, these data demonstrate that NK cells in the donor graft are recruited from the circulation and migrate into the dermis and epidermis where they likely adopt tissue-resident cell properties.

Next, we took genetic approaches to block trNK cell development, and thereby augment the cytotoxic function of cNK cells in the skin. In a cancer setting, enhanced NK cell cytotoxicity is accomplished through the elimination of the immunosuppressive signal from TGFβ (37), which converts cNK to trNK cells (13). WT and Ncr1^{iCre}, Tgfbr2^{fl/fl} mice were transplanted with m157^{tg}, B2m^{-/-} skin grafts; however, no rejection was observed at day 20 post-transplant (FIG. 3D). Likewise, the deletion of transcription factor Hobit, which is required for the development of liver trNK cells (38), in recipient mice did not impact m157^{tg}, B2m^{-/-} skin grafts after 20 days (FIG. 3D). Quantification of the frequency of cNK and trNK cells in the recipient liver and skin grafts revealed increased cNK and decreased trNK cells in m157^{tg}, B2m^{-/-} skin grafts in Ncr1^{iCre}, Tgfbr2^{fl/fl} and Hobit^{-/-} compared with WT recipients (FIG. 3E). Therefore, the signal(s) in the skin that restrict NK cell cytotoxicity are not superseded by exogenous NK cell-stimulating factors, restriction of immunosuppressive TGFβ signaling or prevention of cNK to trNK cell conversion.

### Example 3. m157 in the Absence of Self MHC-I Induces Robust Skin Graft Rejection

Our previous attempts to induce rejection of skin grafts through activation of the Ly49H pathway together with loss of MHC-I failed in a syngeneic transplant system. In order to preserve CD8⁺ T cell functionality while removing NK cell inhibition through the absence of self MHC-I, we generated Ncr1^{iCre}, ROSA^{mT-mG}recipient mice that were first-generation (F1) progeny of B6×BALB/c parents and transplanted them with WT and m157^{tg} B6 skin grafts (FIG. 4A). In this setting, CD8⁺ T cells were still functional through matched MHC-I signaling while NK cells licensed by BALB/c MHC-I lost MHC-I-mediated inhibition against B6 donor skin cells. Although WT skin grafts remained intact in F1 recipients, the majority of m157^{tg} skin grafts were rejected by day 20 (FIG. 4B). The remainder of m157^{tg} grafts were fully rejected by day 60 post-transplant (FIG. 4B). m157^{tg} skin graft size was significantly reduced compared with WT grafts starting from day 16 post-transplant, similar to the timeline of mOVA^{tg} skin graft rejection in WT B6 mice (FIG. 4C). Thus, an NK cell-specific activating ligand in the context of missing-self in F1 recipients induces skin graft rejection; however, this rejection does not initiate within a 48-72-hour window expected of NK cell direct cytotoxicity. Since BALB/c mice do not express Ly49H, the proportion of Ly49H-expressing cNK cells in the B6×BALB/c F1 mice was reduced compared with B6 mice. However, similar to B6 recipient mice (FIG. 2E), the majority of cNK cells that migrated into the m157^{tg} skin graft in B6×BALB/c F1 mice were Ly49H⁺ that downregulated Ly49H receptor expression upon m157 engagement in the donor skin. This data indicates that despite fewer circulating Ly49H⁺ cNK cells in B6×BALB/c F1 mice, these cells were fully active against m157-expressing B6 skin graft.

Significant increases in the number of NKp46-GFP⁺ NK, CD4⁺T and CD8⁺ T cells were found in m157^{tg} skin grafts at day 10 post-transplant (FIG. 4D). Quantifying the immune cell infiltrates over time revealed that significant numbers of NKp46-GFP⁺ NK cells appeared in m157^{tg} skin grafts at day 5 post-transplant while CD4⁺T and CD8⁺ T cells infiltrated m157^{tg} skin grafts with a delay (FIG. 4E). Recruitment of NK and T cells into the donor epidermis was also detected at later time points (fig. S5D). To determine whether m157^{tg} graft rejection in F1 recipients was NK cell-dependent, we transplanted m157^{tg} skin onto Ncr1^{iCre}, ROSA^{DTR} F1 recipient mice and treated them with diphtheria toxin (DT) and anti-NK1.1 antibody over 18 days to deplete NK cells. ROSA^{DTR} F1 recipient mice administered with DT and control antibody were used as controls. Depletion of NK cells in F1 recipients prevented rejection of m157^{tg} skin grafts (FIG. 4F). Importantly, depletion of T cells using CD4^{Cre}, ROSA^{DTR} F1 mice together with DT and CD4 and CD8 depleting antibodies also prevented rejection of m157^{tg} skin grafts (FIG. 4F). Tracking the survival of m157^{tg} skin grafts in Ncr1^{iCre}, ROSA^{DTR} mice after the termination of DT plus anti-NK1.1 antibody treatment demonstrated that recovery of NK cells in recipient mice led to complete graft rejection by day 60 post-transplant (FIG. 4G). Indeed, m157^{tg} skin grafts began to recede and develop scabs approximately 20 days after NK cell depletion was stopped (FIG. 4G). These data demonstrate that NK and T cells are required for the rejection of m157^{tg} skin grafts in the context of missing-self. The NK cell-specific nature of the m157 activation signal and the timeline of NK, and later, T cell infiltration into the skin suggests that activated NK cells may have recruited T cells as effectors to reject the skin graft.

### Example 4. CNK Cells Switch their Function from Cytotoxicity to Chemokine/Cytokine Production as Soon as they Exit the Circulation

To define the function of NK cells that infiltrated m157-expressing skin grafts, we examined the transcriptional profiles of cNK cells in the circulation (spleen and blood), cNK cells recently emigrated from the circulation into m157-expressing skin grafts, dpNK and trNK cells in the skin grafts and trNK cells in the recipient skin (FIG. 11A). This strategy enabled us to identify pathways that were up or downregulated in cNK cells early during their entry into the skin microenvironment. Strikingly, we discovered a dramatic switch in cNK cells' profile as they entered the skin grafts, which was highlighted by downregulation of cytotoxicity-related genes and upregulation of chemokines and inflammatory cytokines (FIG. 5A and FIG. 11B). Ccl1, Ccl2, Cxcl2, Cxcl9 and Cxcl10 were significantly upregulated in cNK cells in the skin graft compared to cNK cells in circulation (FIGS. 5, A and B). Likewise, Il1a, Il1b and Tnf were upregulated in donor skin cNK cells (FIGS. 5, A and B). No upregulation was seen in Ifng, Gzma or Gzmb expression, and Prf1 (perforin), Klrk1, Ccl5, Eomes and Tbx21 (T-bet) were significantly downregulated in donor skin cNK cells compared with their baseline expression in circulating cNK cells (FIGS. 5, A and B). Based on RNA-Seq data and confirmed by flow cytometry, we identified TGFβRII, IL-4Rα, CCR7 and TIGIT as novel markers to distinguish cNK cells in the skin from circulating cNK cells (FIGS. 11, C and D). Together, these data demonstrate that upon entry into the skin, cNK cells undergo a drastic change in their function, which involves downregulating their cytotoxic program while boosting their ability to provide ‘help’ to neighboring immune cell populations through the production of chemokines and inflammatory cytokines.

Considering the significant upregulation of Tigit, Pdcd1 and Ctla4 in cNK cells entering the skin grafts (FIGS. 5, A and B), we examined the impact of immune checkpoint blockade on the rejection of m157-expressing skin grafts (FIG. 11E). PD-1, TIGIT or TIGIT/PD-1/CTLA4 triple antibody blockade did not result in rejection of m157^{tg} or m157^{tg}, B2m^{-/-} skin grafts in WT B6 recipients (FIGS. 11, F and G). Further, TIGIT/PD-1/CTLA4 triple antibody blockade did not accelerate the rejection of m157^{tg} skin grafts in F1 recipients (FIG. 11G), indicating the persistent lack of NK cell direct cytotoxicity in the skin grafts.

### Example 5. ECM Proteins as Potential Modulators of cNK Cell's Effector Functions in the Skin

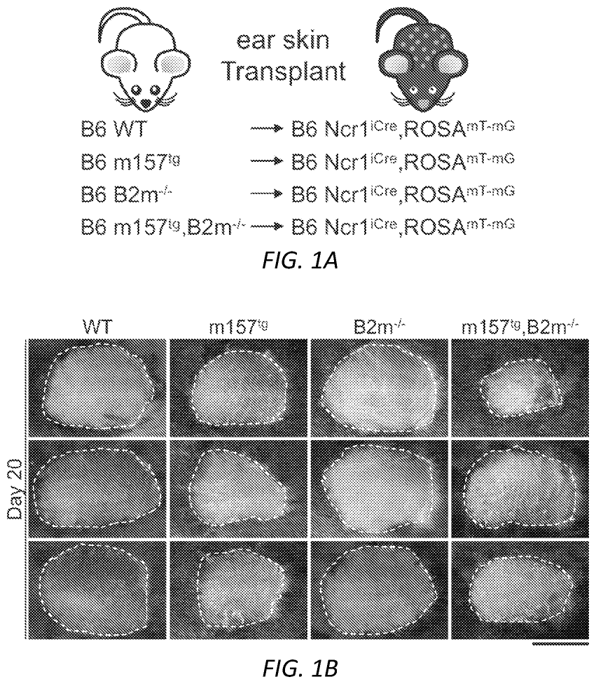

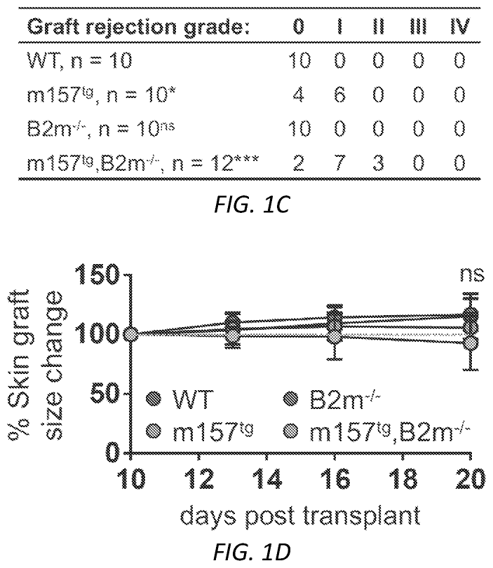

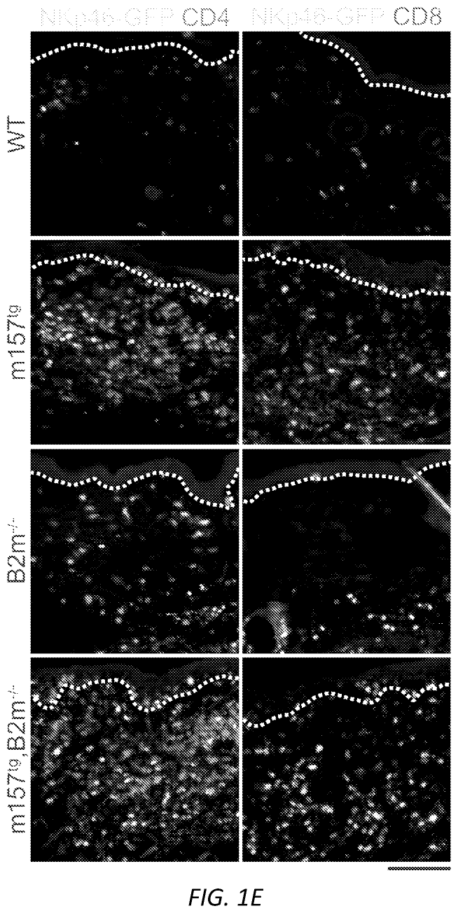

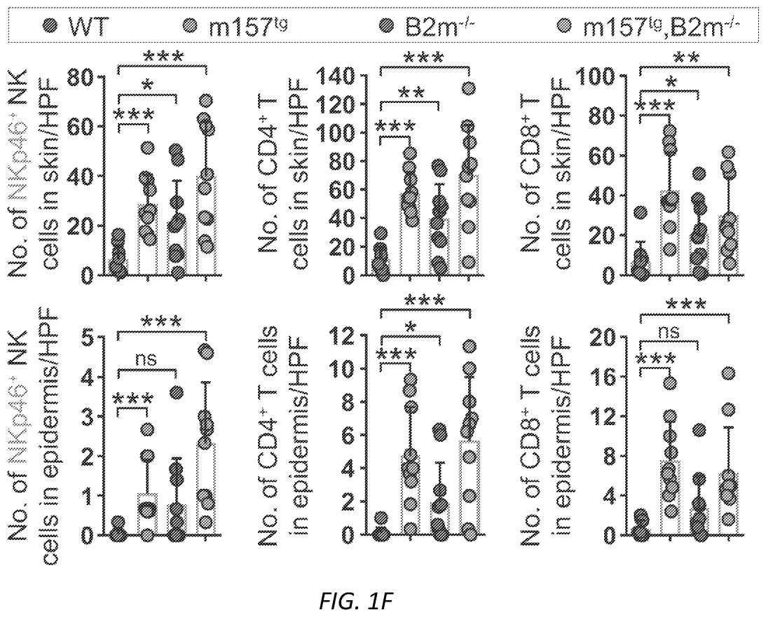

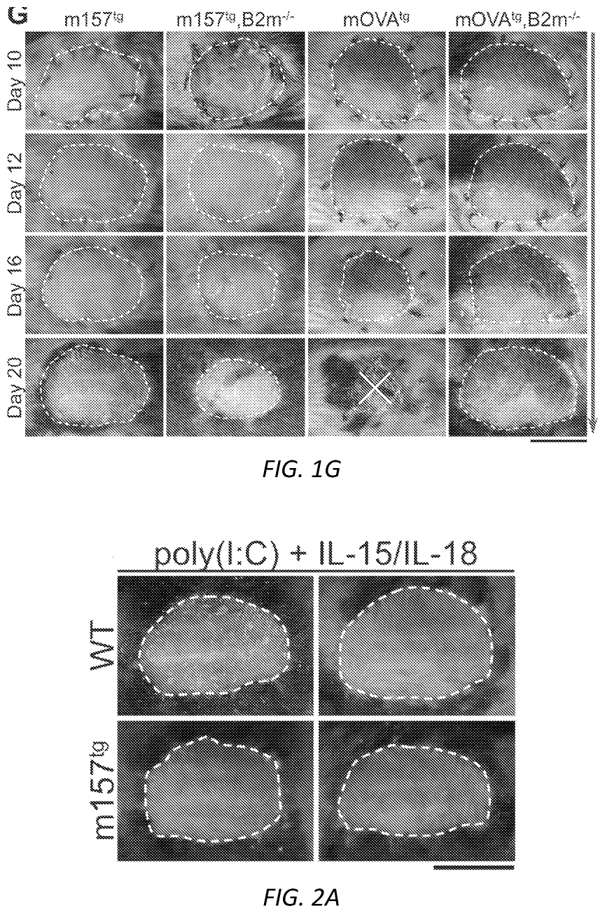

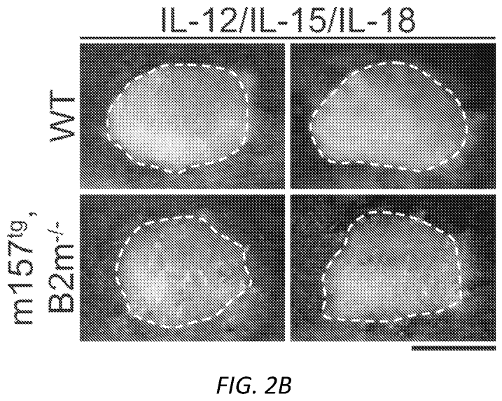

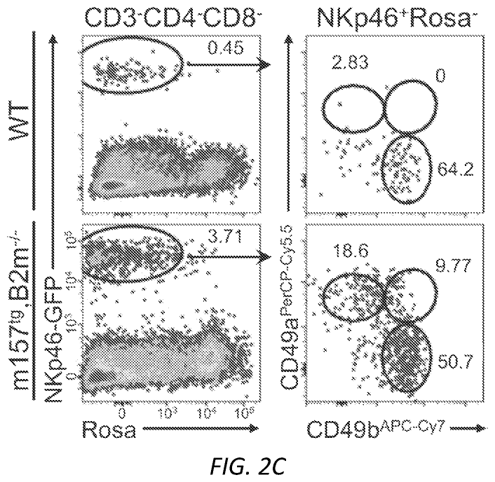

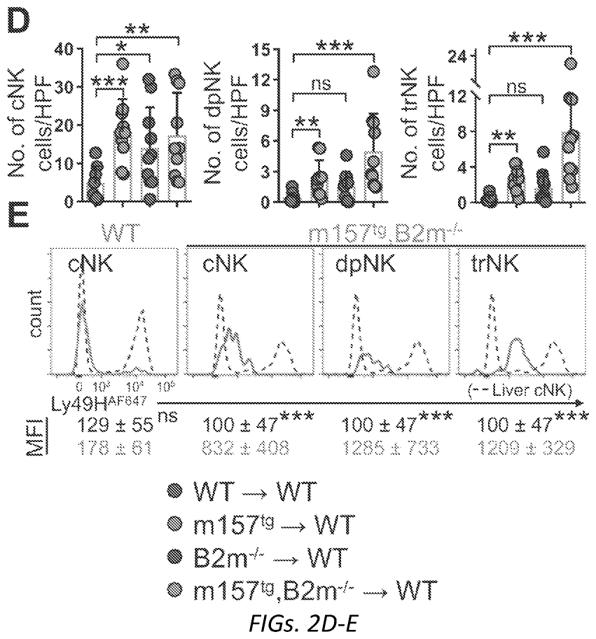

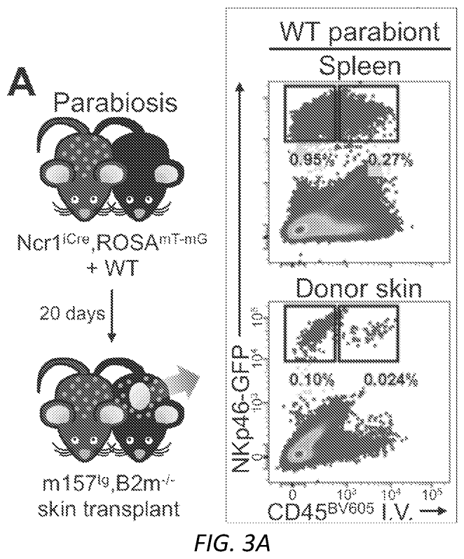

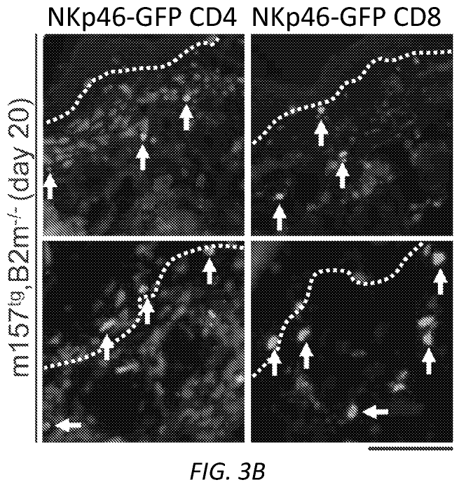

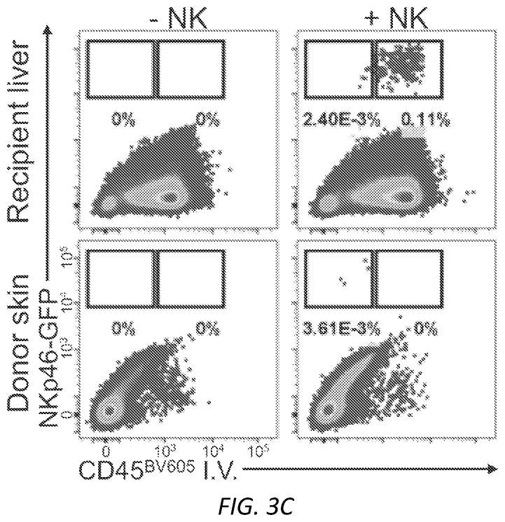

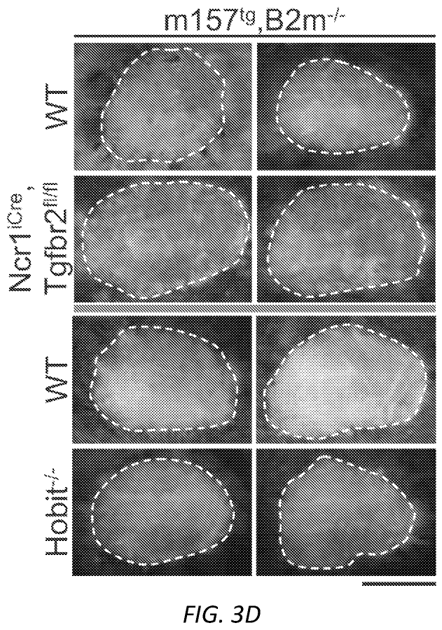

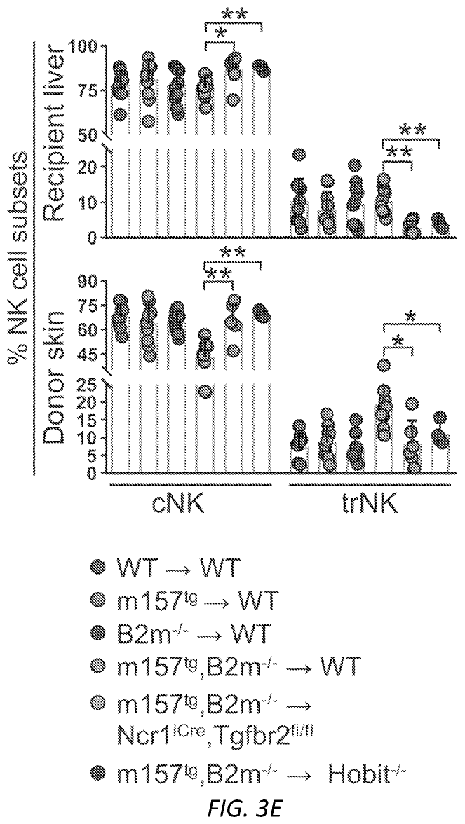

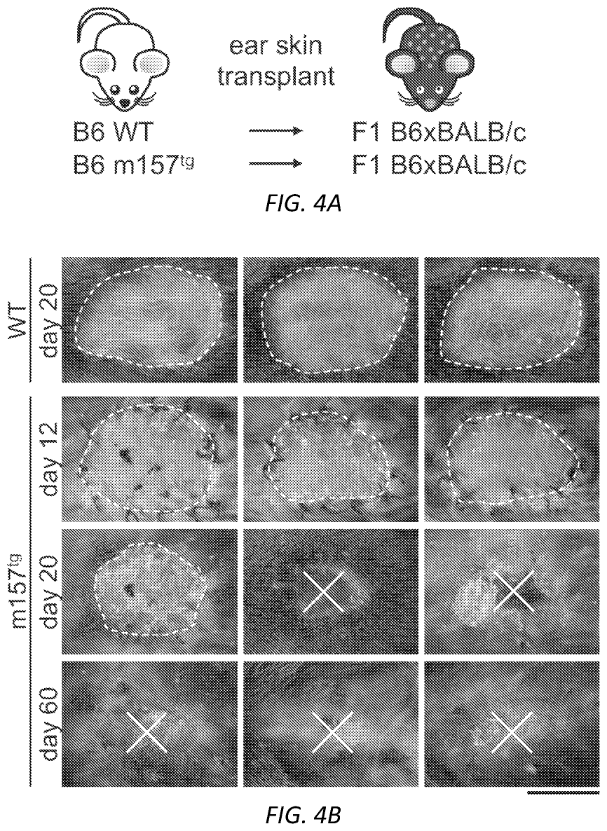

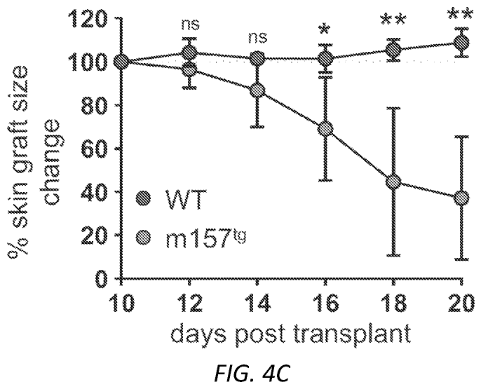

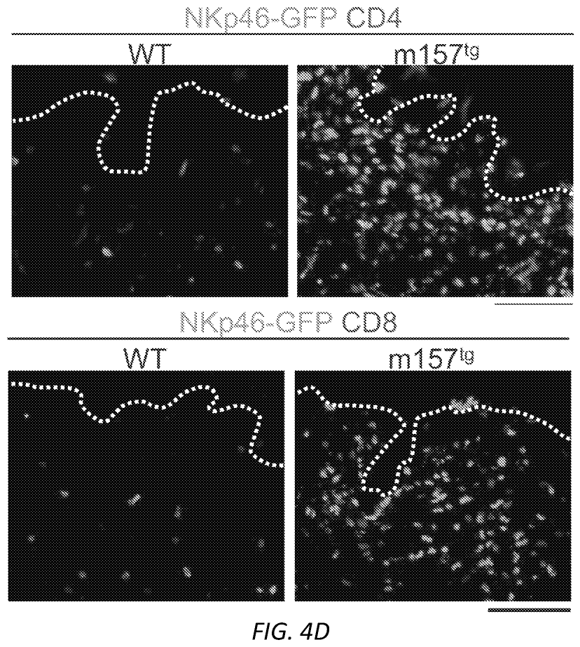

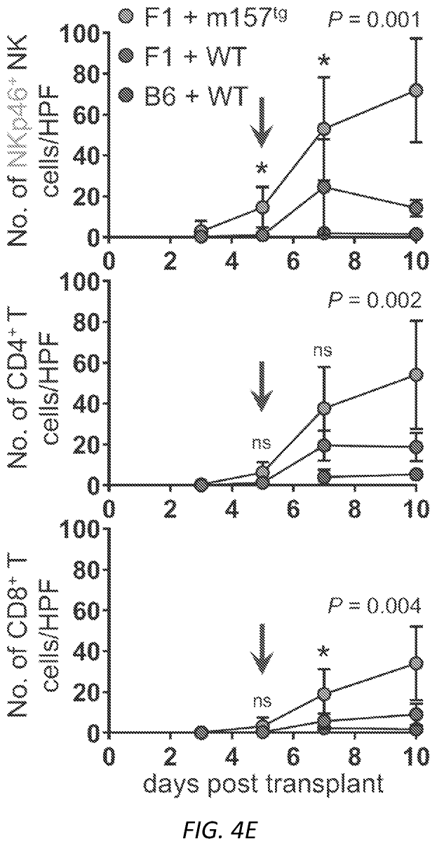

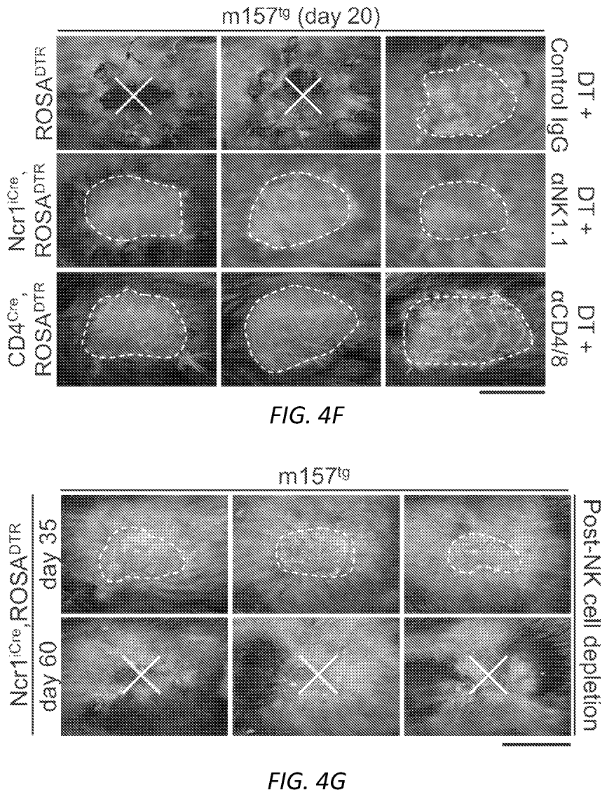

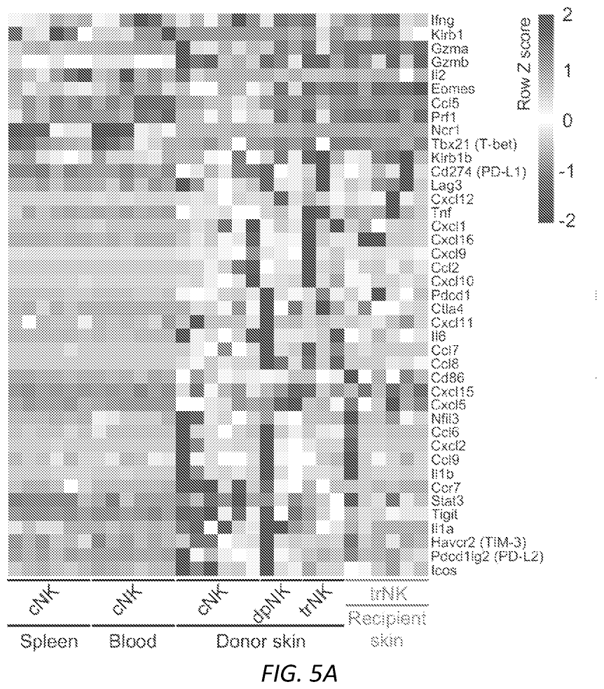

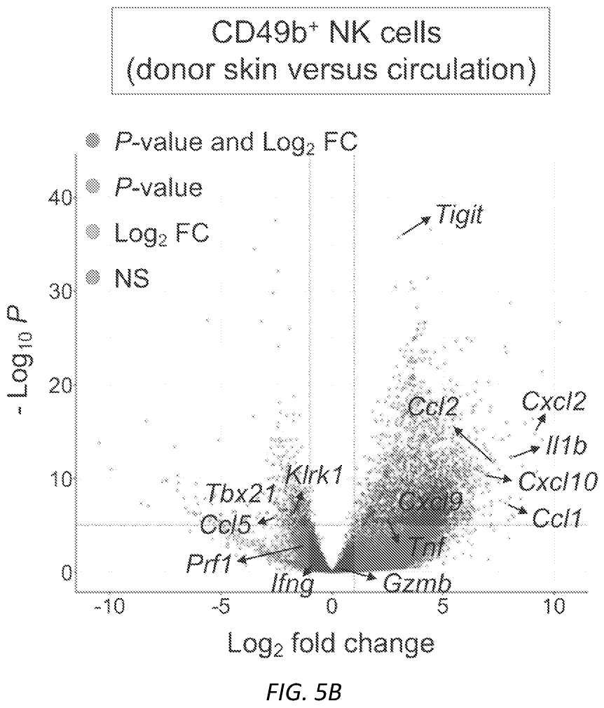

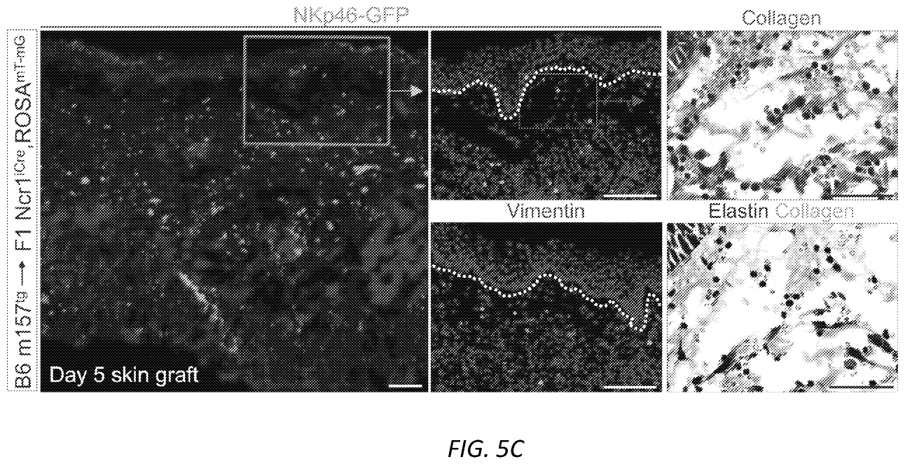

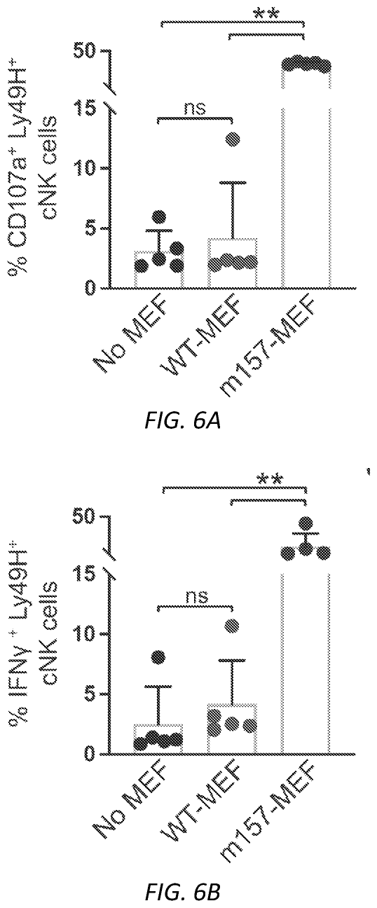

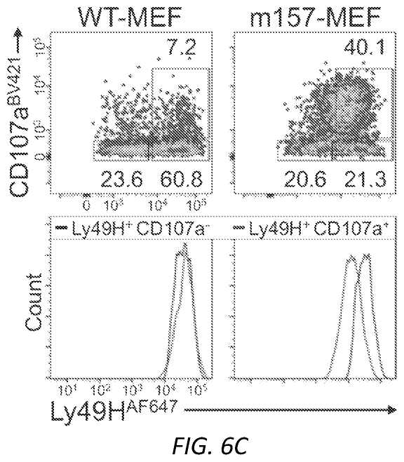

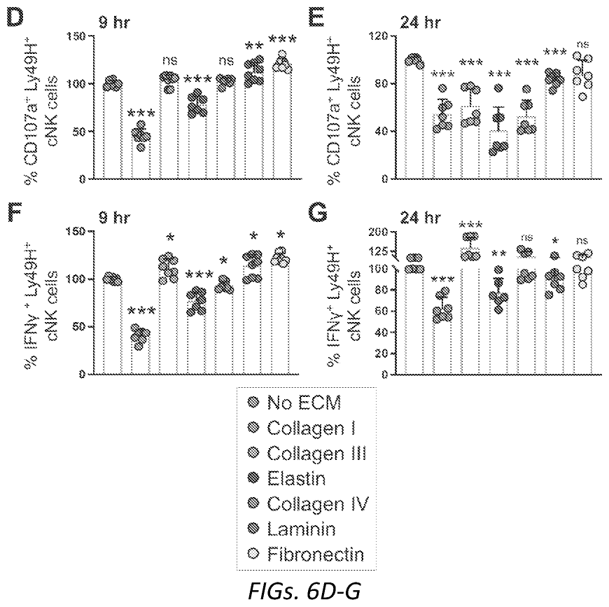

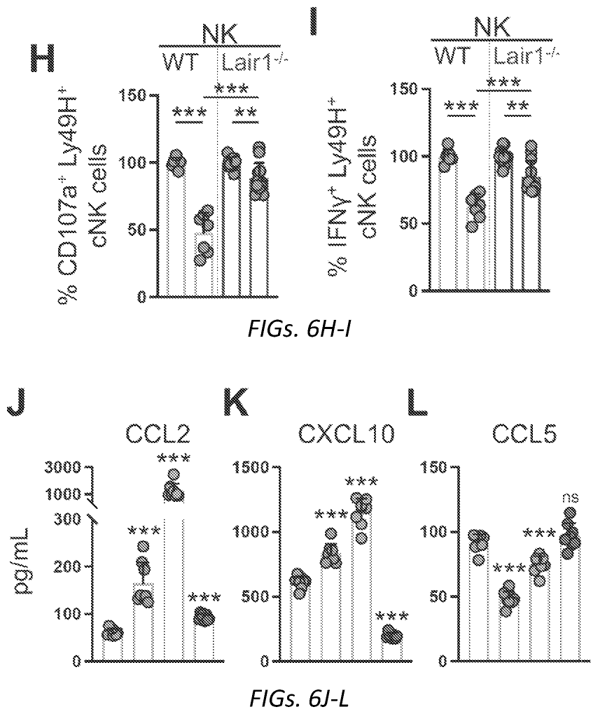

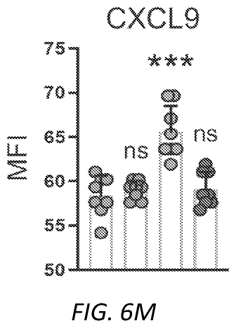

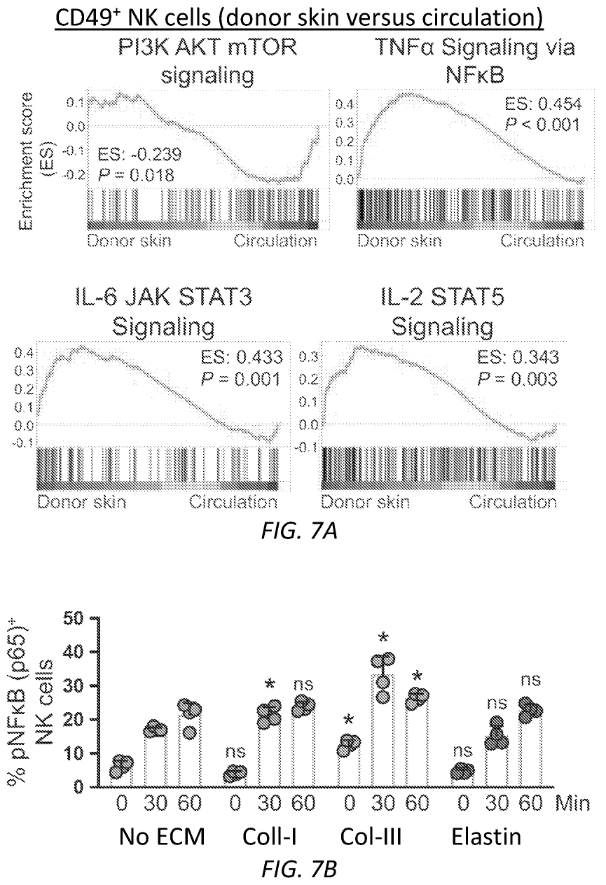

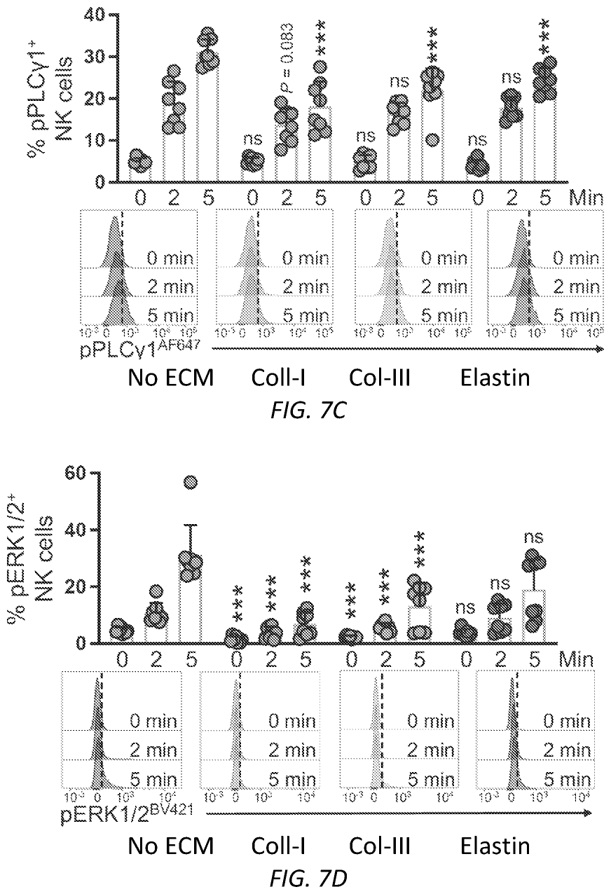

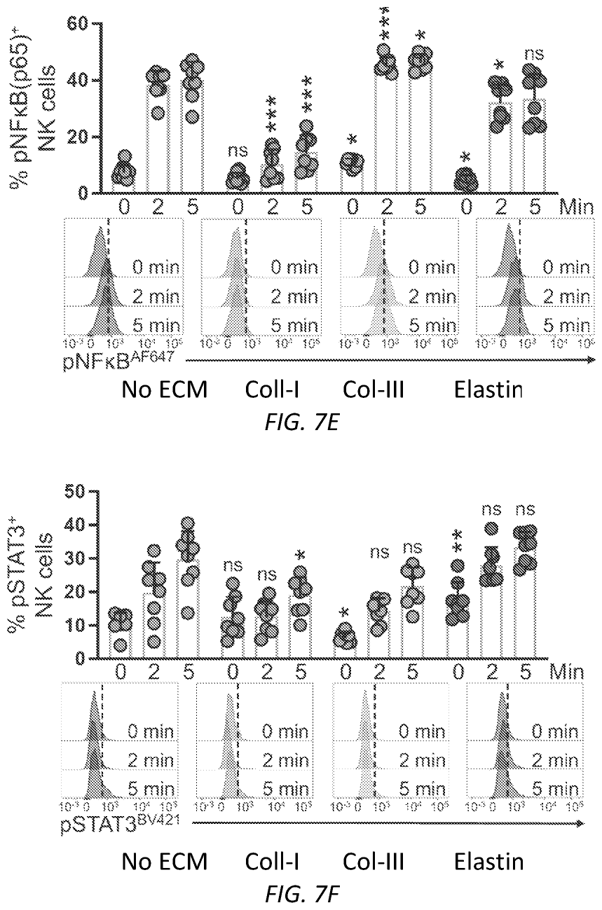

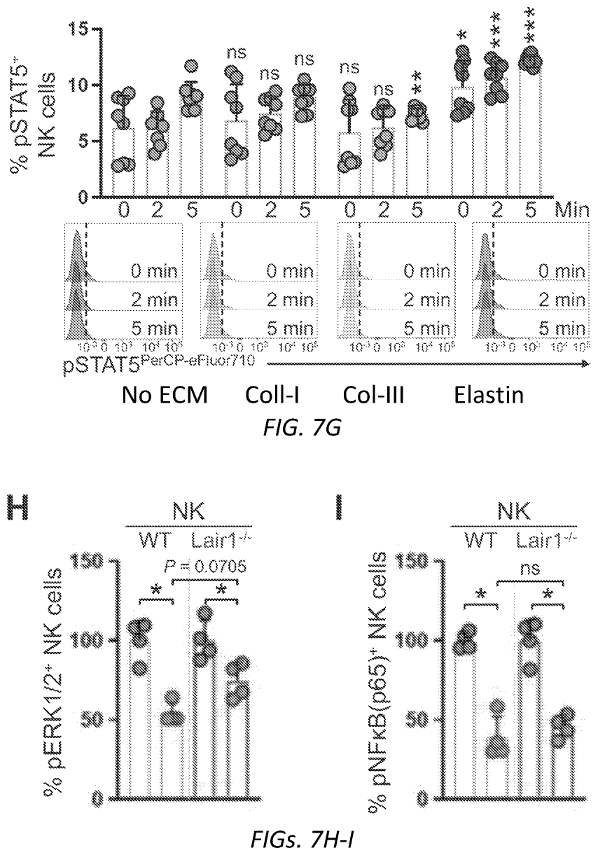

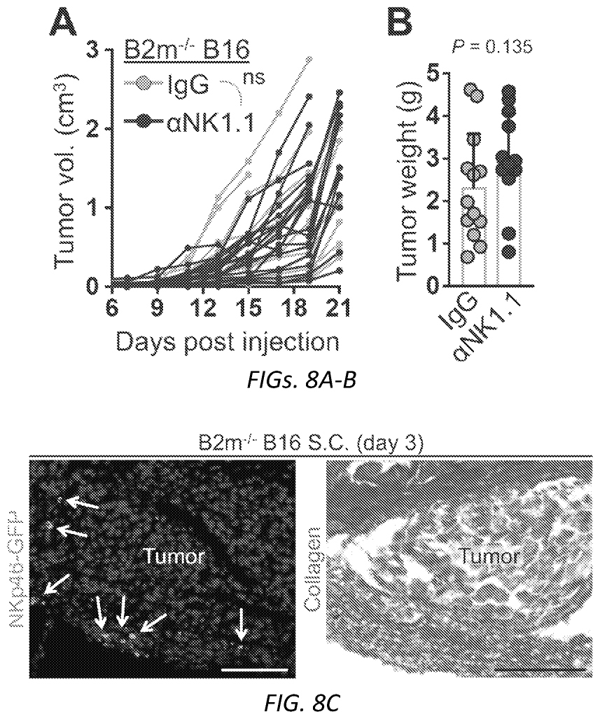

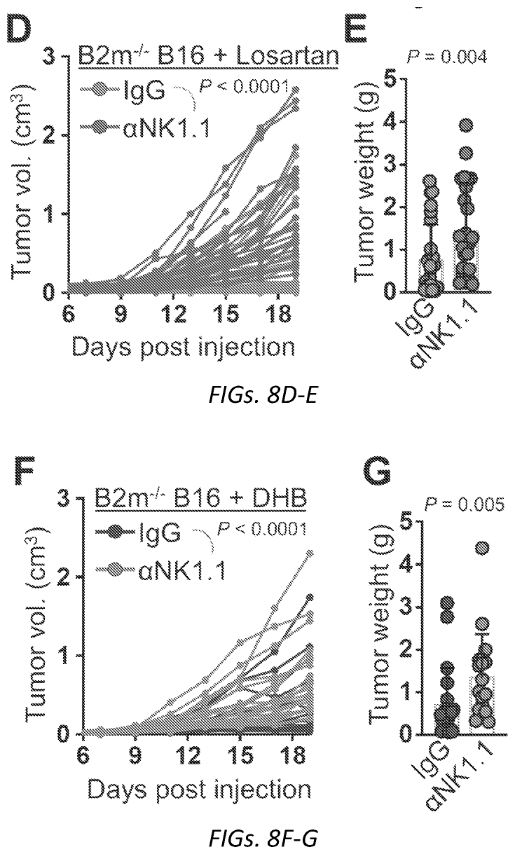

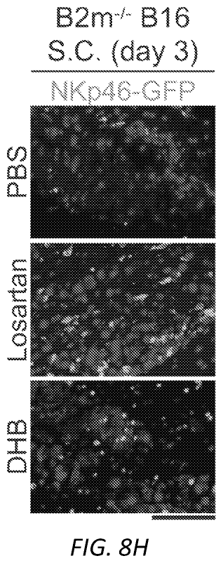

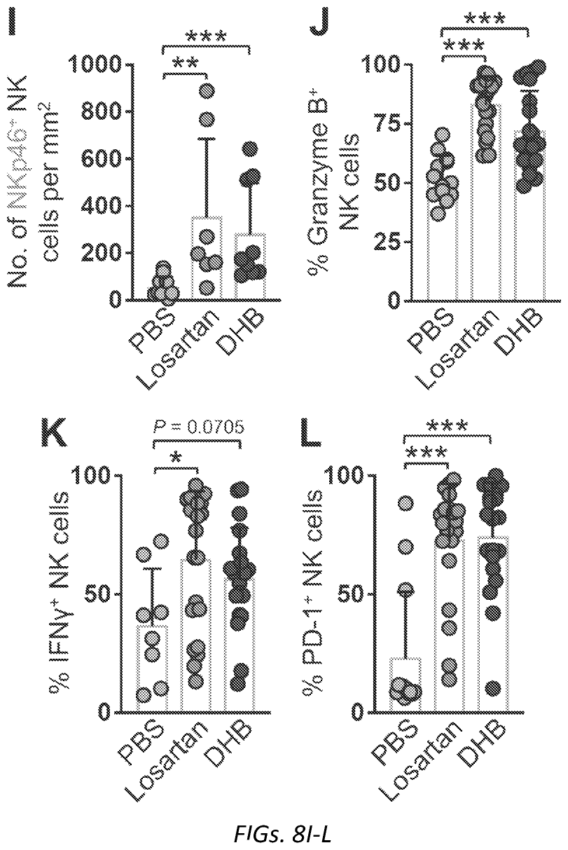

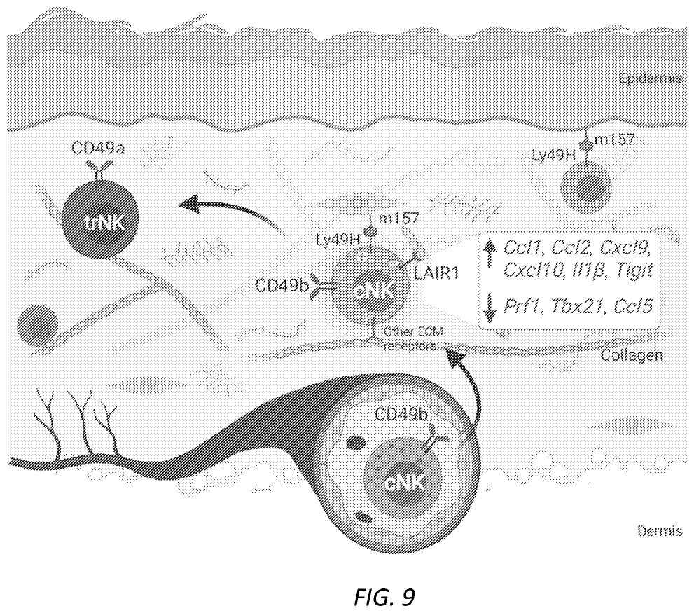

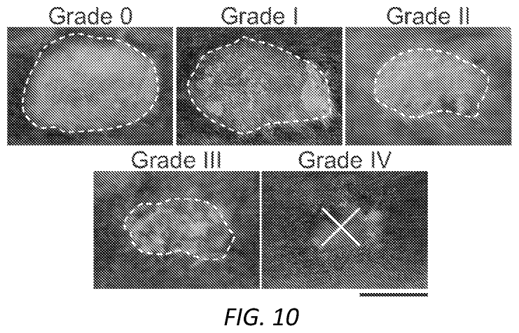

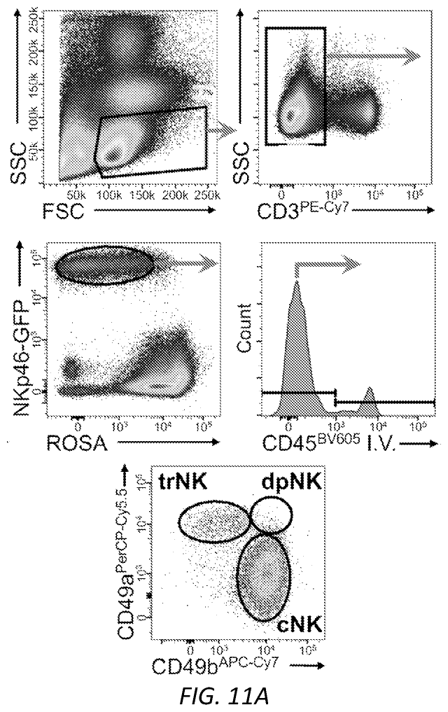

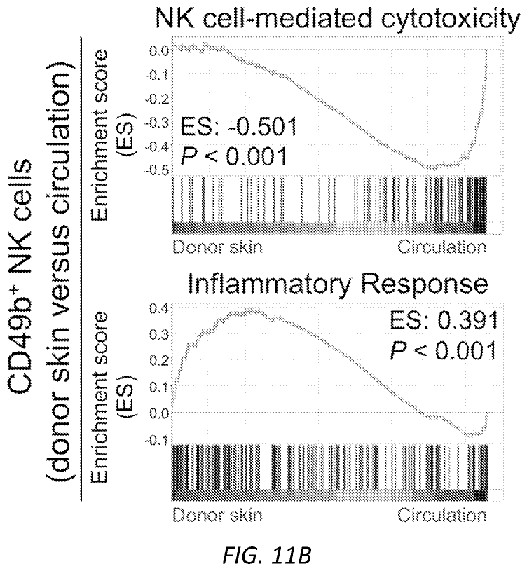

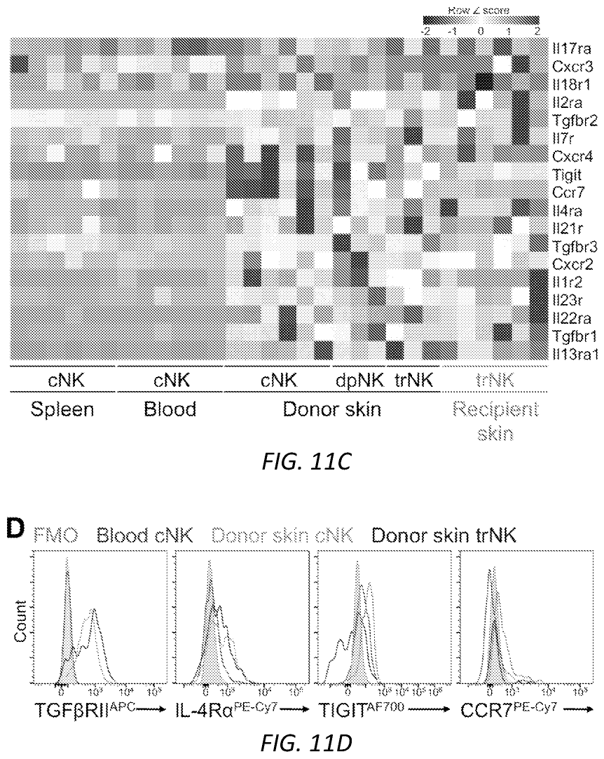

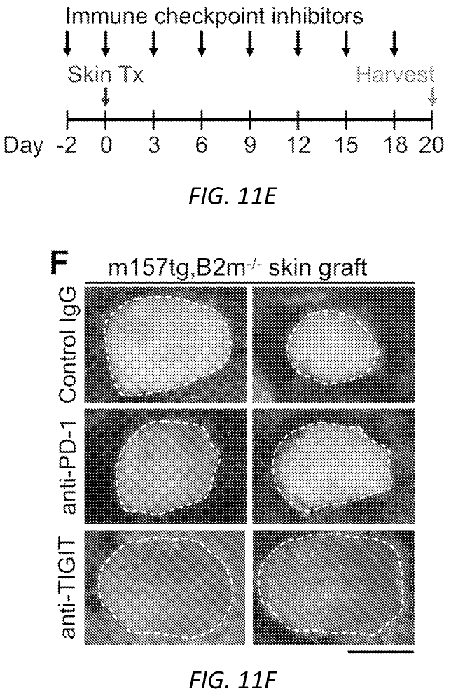

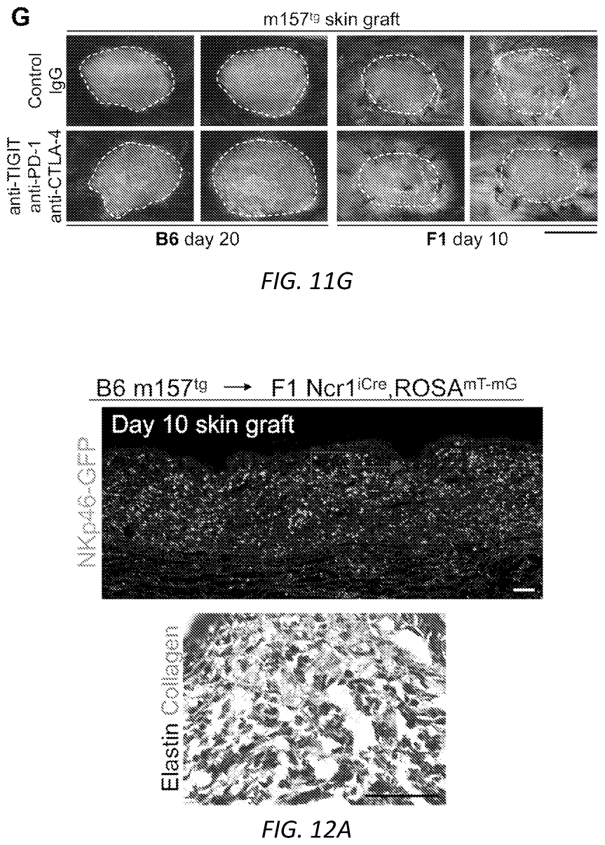

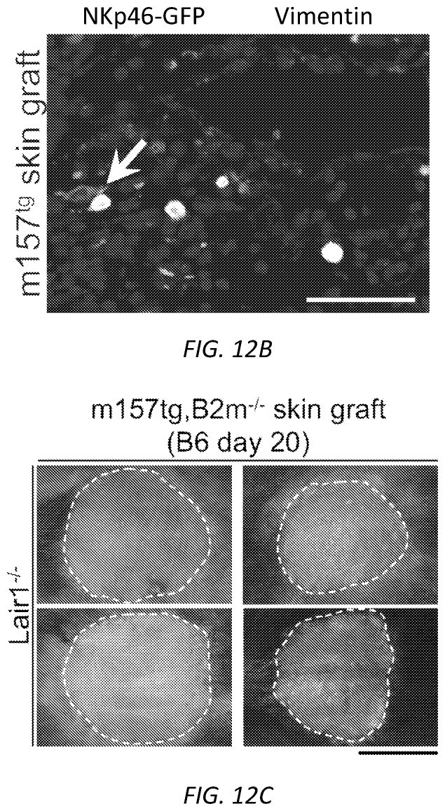

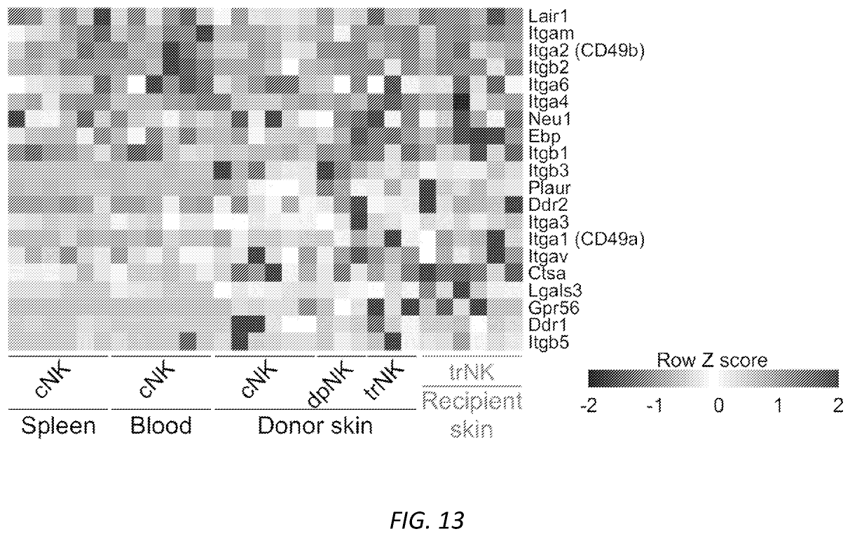

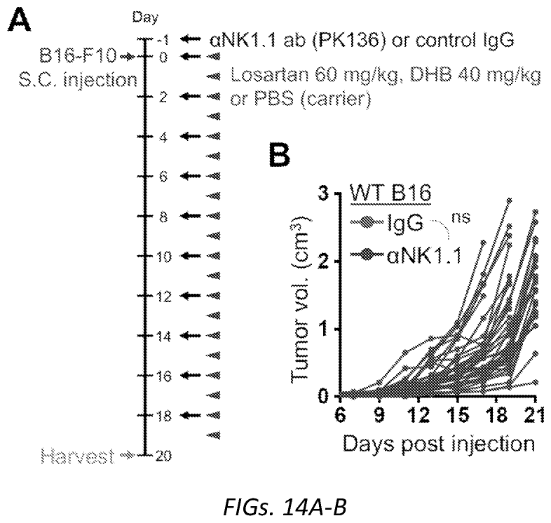

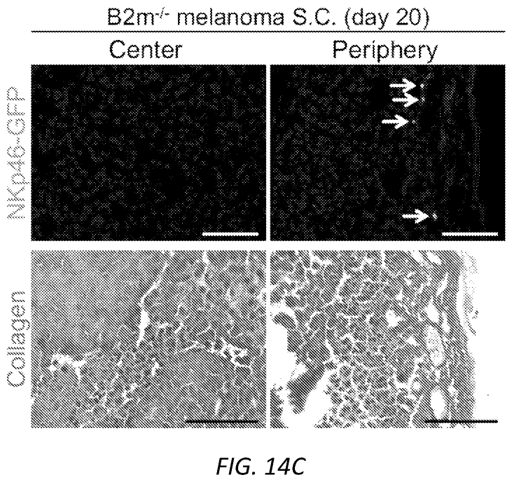

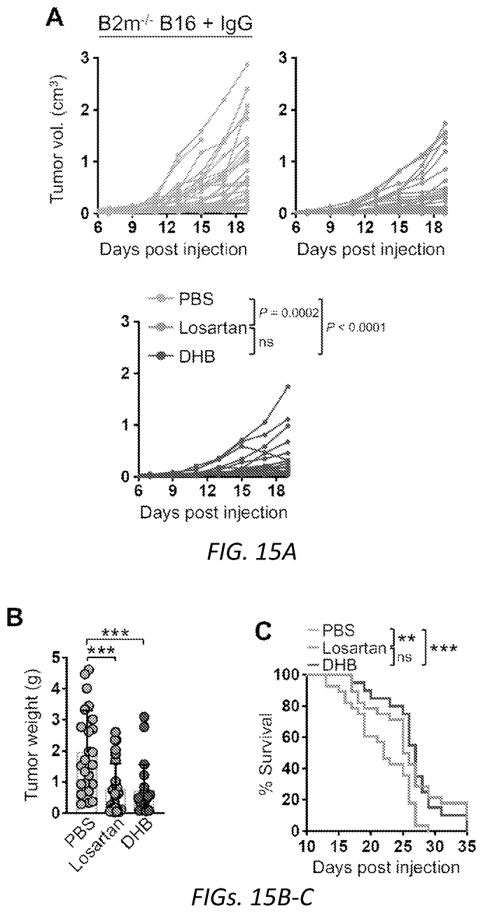

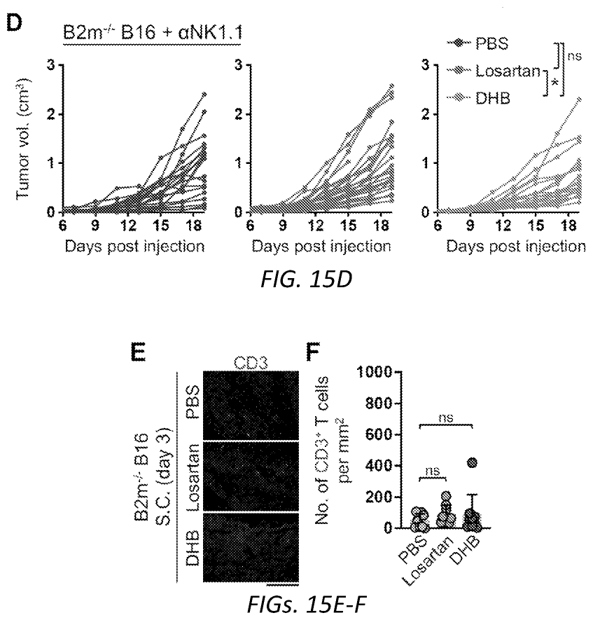

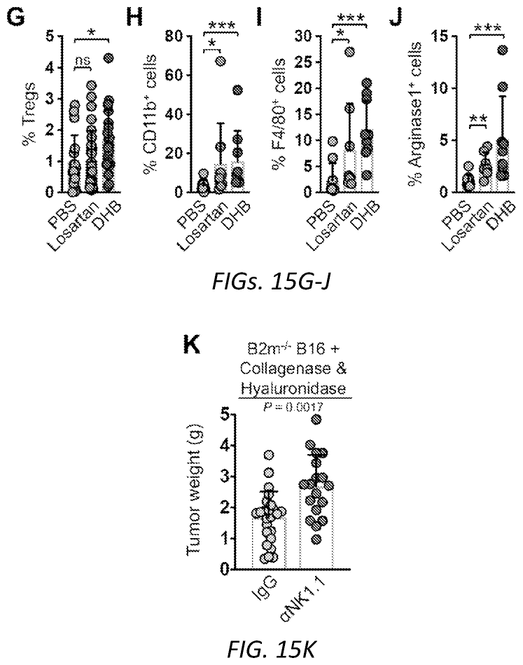

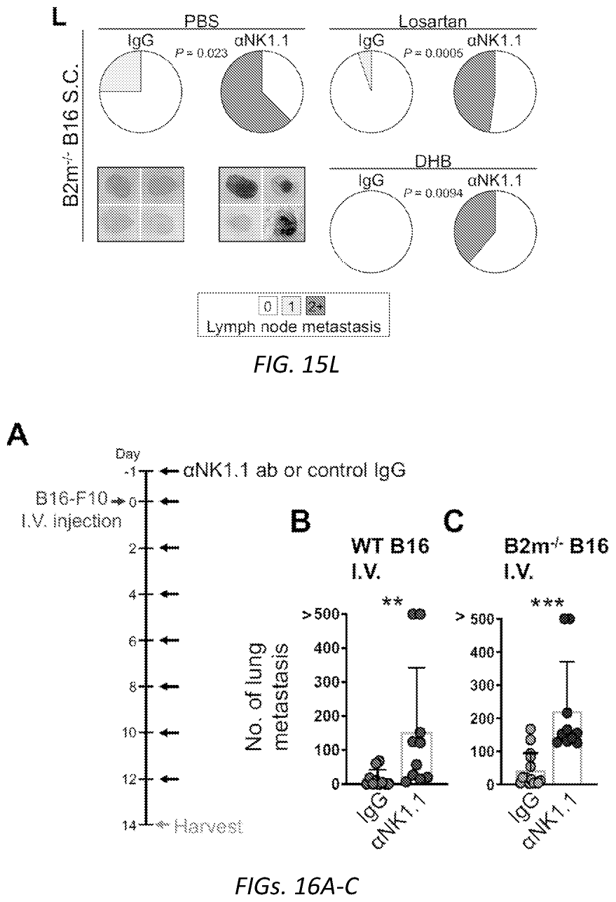

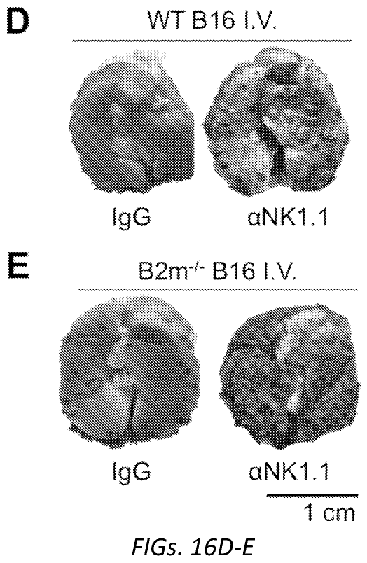

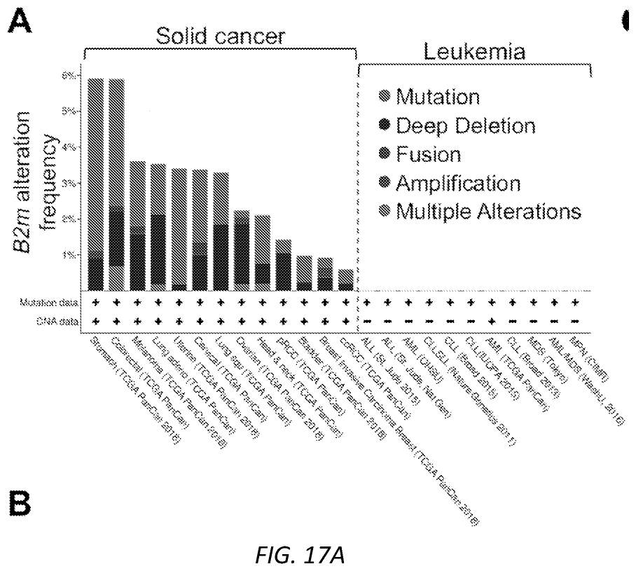

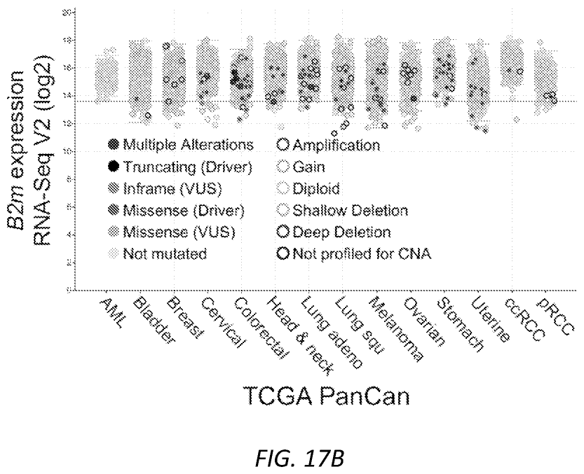

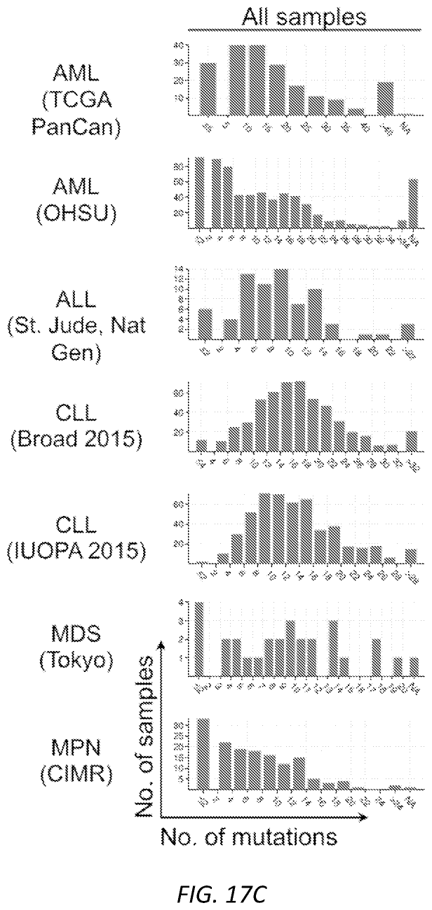

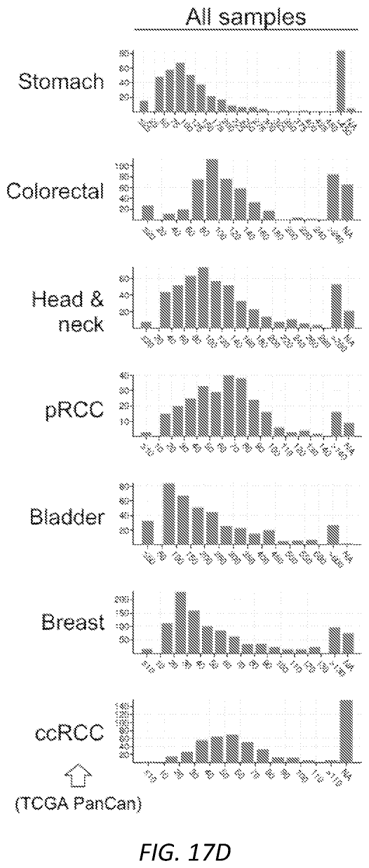

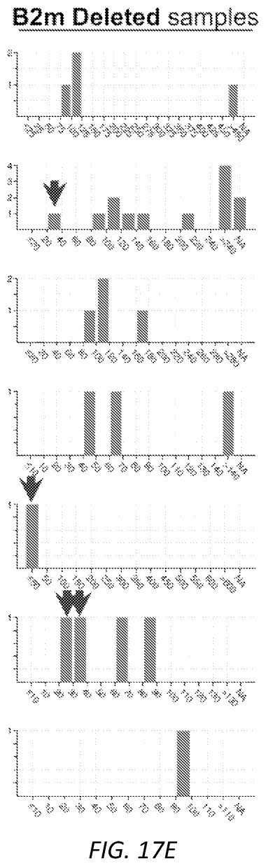

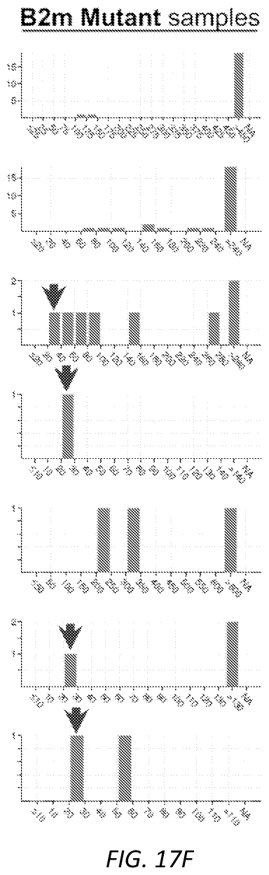

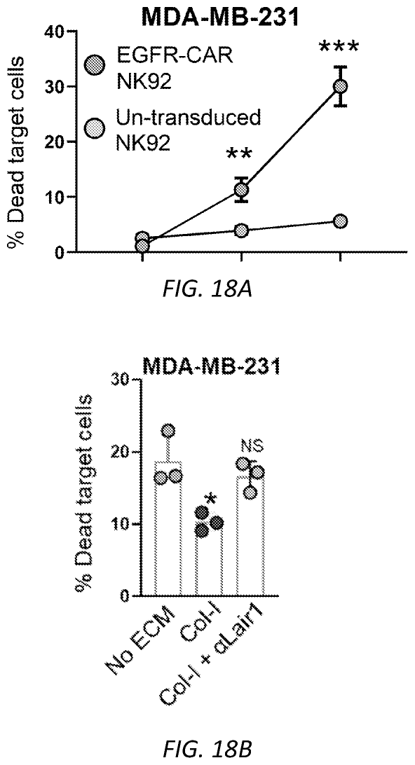

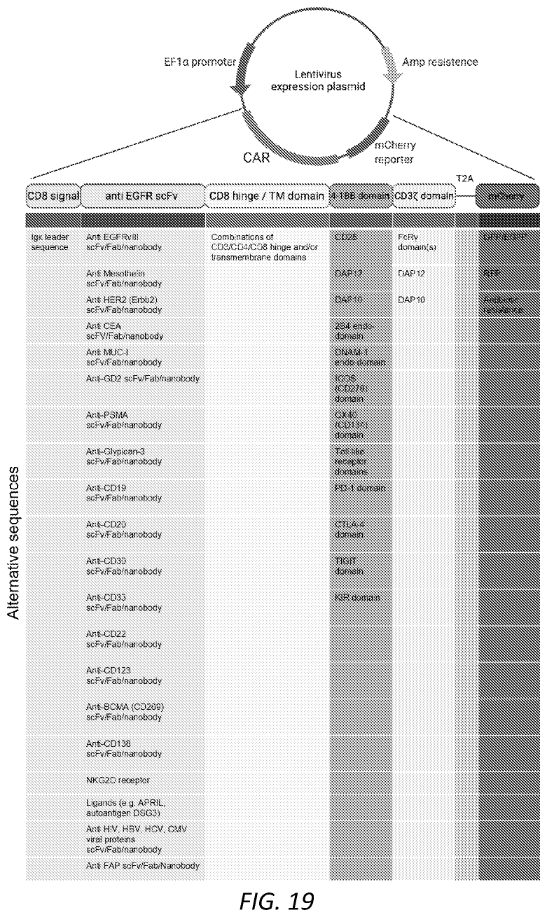

To identify the mediator(s) of NK cell functional switch in the skin, we examined the components of the skin microenvironment that interacted with NK cells as soon as they entered the skin grafts at day 5 post-transplant (FIG. 4E). NK cells infiltrated broadly into m157^{tg} dermal ECM, surrounded by collagen and to a lesser extent elastin (FIG. 5C and FIG. 12A). In addition, occasional NK cells were detected contacting dermal fibroblasts at day 5 post-transplant (FIG. 5C and FIG. 12B). To elucidate the impact of ECM proteins and fibroblasts on cNK cell cytotoxicity, we examined splenic cNK cell degranulation and cytokine production in co-culture assays. WT mouse embryonic fibroblasts (WT-MEF) did not block Ly49H⁺ cNK cell degranulation or IFNγ production in response to IL-12 and IL-15 stimulation (FIGS. 6, A and B). Furthermore, fibroblasts expressing m157 (m157-MEF) markedly induced Ly49H⁺ cNK cell degranulation and IFNγ production, which was associated with Ly49H downregulation (FIG. 6, A-C). In contrast, major dermal ECM proteins, collagen I, III and elastin, potently blocked Ly49H⁺ cNK cell degranulation after 9 and 24 hours co-culture with m157-MEF (FIGS. 6, D and E). Collagen I and elastin also suppressed IFNγ production by Ly49H⁺ cNK cells exposed to m157-MEF (FIGS. 6, F and G). On the other hand, major epidermal basement membrane proteins, collagen IV, laminin and fibronectin, played a minor role in altering NK cell functions (FIG. 6, D-G). Leukocyte Associated Immunoglobulin Like Receptor 1 (LAIR1) is an inhibitory receptor expressed by NK cells that binds collagens (39, 40). Deletion of Lair1 partially rescued collagen I-induced blockade of Ly49H⁺ cNK cell degranulation and IFNγ production upon in vitro exposure to m157-MEF (FIGS. 6, H and I). However, global Lair1 deletion failed to induce the rejection of m157^{tg}, B2m^{-/-}skin graft in Lair1^{-/-} mice (FIG. 12C). This suggests that the partial LAIR1 dependency observed in our in vitro study may be compensated by complex interplay between NK cells and other ECM proteins in the skin. The close linkage between Ncr1 and Lair1 genes on chromosome 7 in mice prevented us from generating Ncr1^{iCre}, Lair1^{fl/fl} mice to examine the NK cell-specific role of LAIR1 in vivo. Strikingly, Collagen I and III significantly induced CCL2 and CXCL10 but suppressed CCL5 release by splenic cNK cells cultured with m157-MEF (FIG. 6, J-L). Collagen III also induced CXCL9 release by cNK cells (FIG. 6M).

To gain a greater insight into the underlying molecular mechanisms linking the ECM to changed NK cell functionalities, we investigated the signaling pathways that were associated with the switch in cNK cells' profile from cytotoxicity to an inflammatory response (fig. S6B). cNK cells entering the skin downregulated PI3K-AKT pathway while upregulating NF□B, STAT3 and STAT5 signaling pathways compared with circulating cNK cells (FIG. 7A). Interestingly, we discovered that Collagen III significantly induced phospho-NF□B (p65) in splenic cNK cells at baseline, which was highly amplified after 30- and 60-minutes exposure to m157-MEF (FIG. 7B). To examine the impact of ECM proteins on NK cell signaling upon activation in a time-controlled setting that excluded the possibility for ECM proteins to physically block activating ligand-receptor interactions, we activated NK cells with H₂O₂ in the presence of collagens and elastin (41). Notably, collagens and elastin markedly reduced cytotoxicity-associated pPLCγ1 and pERK1/2 levels in the NK cells (FIGS. 7, C and D). In contrast, collagen III increased pNFκB in NK cells at baseline and after 2- and 5-minute exposure to H₂O₂ (FIG. 7E). Interestingly, elastin increased pSTAT3 at baseline and highly induced pSTAT5 at baseline and after 2- and 5-minutes exposure to H₂O₂ (FIGS. 7, F and G). Collagen I showed a suppressive effect on pNFκB and pSTAT3 (FIGS. 7, E and F). Loss of Lair1 in NK cells partially reversed pERK1/2 suppression by collagen I but had no impact on NFκB phosphorylation after 2 minutes exposure to H₂O₂ (FIGS. 7, H and I). Consistent with their critical role in regulating cNK cell function, we found the expression of multiple ECM protein receptors on cNK cells entering the skin microenvironment (FIG. 13). These findings indicate that ECM proteins including collagen I, III and elastin are potent regulators of cNK cell function, which collectively may lead to reduced cytotoxicity and increased chemokine and cytokine production by NK cells as they enter the peripheral tissues.

### Example 6. ECM Proteins Suppress NK Cell Antitumor Response in the Skin

Considering the essential role of ECM proteins in skin homeostasis and wound healing, our attempts to remove or degrade ECM proteins in the skin transplantation system led to the failure of skin engraftment. Thus, we examined the role of ECM proteins in suppressing NK cell cytotoxicity against cancer cells in the skin. We subcutaneously (S.C.) injected B2m^{-/-} B16-F10 melanoma cells (B16) into the flanks of WT mice and treated them with control IgG or anti-NK1.1 antibody to determine if NK cells inhibited the growth of MHC-I-deficient tumors in the skin (FIG. 14A). Similar to WT melanoma, NK cell depletion did not impact the growth or terminal weight of B2m^{-/-} melanoma in the skin (FIGS. 8, A and B, and FIG. 14B). NK cells infiltrated B2m^{-/-} melanoma as early as day 3 post-tumor injection but were embedded in a collagen-rich ECM surrounding the tumor foci (FIG. 8C and FIG. 14C). To investigate the role of tumor collagen deposition in suppressing NK cell immunity against melanoma in the skin, we treated mice that received B2m^{-/-} B16 S.C. with losartan (60 mg/kg) to block collagen synthesis or collagen prolyl 4-hydroxylase inhibitor, 3,4-dihydroxybenzoic acid (DHB, 40 mg/kg), which blocks collagen assembly into a triple helix (FIG. 14A) (42, 43). Importantly, losartan and DHB treatment protected the mice from B2m^{-/-} melanoma growth in the skin in an NK cell-dependent manner, which led to a significant survival benefit (FIG. 8, D-G and FIG. 15, A-D). NK cells highly infiltrated the tumor parenchyma in losartan and DHB-treated mice and showed markedly elevated expression of granzyme B, IFNγ and PD-1 (FIG. 8, H-L). Unlike NK cells, Losartan and DHB treatment did not induce T cell recruitment into the B2m^{-/-} B16 tumors (FIGS. 15, E and F). Furthermore, the proportions of tumor infiltrating regulatory T cells (Tregs), macrophages and myeloid derived suppressor cells (MDSCs) remained unaltered or increased following Losartan and DHB treatments (FIG. 15, G-J). To further confirm that the NK cell immunity unleashed upon losartan and DHB treatment was related to their effects on blocking collagen deposition in the tumor, we treated a group of mice that received B2m^{-/-} B16 S.C. with collagenase and hyaluronidase mixture and found a similar dependency of tumor growth on NK cells (FIG. 15K). Interestingly, NK cell depletion in PBS (carrier control), losartan and DHB-treated mice resulted in lymph node metastasis of B2m^{-/-} melanoma, suggesting a role for NK cells in the clearance of metastasizing melanoma cells (FIG. 15L). Consistent with this effect, and in contrast to the lack of NK cell's ability to control melanoma in the skin, NK cells effectively killed intravenously (I.V.) injected WT and B2m^{-/-} B16 cells and suppressed melanoma lung metastasis (FIGS. 16A-E). Collectively, these data indicate strong NK cell killing of MHC-I-deficient tumor cells in the circulation, which is likely blocked by ECM proteins in peripheral tissues.

To translate our fundamental findings to human cancers, we analyzed the TCGA and other publicly available datasets for the frequency of B2Mgene alteration and mutations in multiple types of solid cancers and leukemias. While B2Mgene mutations and deletion were found across solid cancer types including those with low mutational burden, leukemias did not carry any aberrations in the B2M gene (FIGS. 17, A and B). Furthermore, B2M gene expression in acute myeloid leukemia (AML) samples was consistently high and showed the least variance compared with solid cancer types (FIG. 17C). These findings indicate a remarkable resistance to downregulation of the B2Mgene in blood cancers even when confronted with CD8⁺ T cell pressure. Thus, circulating cytotoxic NK cells likely eliminate leukemias that lose MHC-I while solid cancers that downregulate MHC-I in peripheral tissues are protected due to the impaired NK cell cytotoxicity caused by ECM proteins.

### Example 7. Exemplary Protocol to Generate Lair1 Knockout NK92MI and EGFR-CAR NK92MI Cells for Adoptive Therapy

### Generation of EGFR CAR-NK92 Cells

To generate EGFR CAR containing lentivirus, HEK293T cells were transfected with lentivirus expression plasmid (FIG. 19) and lentivirus packaging plasmids in OptiMEM medium using Lipofectamin 3000 reagent as per the manufacturer's protocol.

Lentivirus containing supernatant (S/N) was collected at 24 and 48 hours after transfection and stored at 4° C. Following 48-hour collection, S/N was filtered using 0.45 μm filters and was ultracentrifuged at 25000 RPM for 1.5 hours to concentrate the lentivirus particles. Lentiviral pellet was resuspended in appropriate volume and stored at −80C until further use. Lentivirus titer was calculated using SupT1 cells by serial dilution and detecting mCherry reporter expression by flow cytometry.

NK92MI cells were transduced with EGFR CAR lentivirus at MOI 5 by spinofection method (1000 g, 1.5 hrs at 32C) in the presence of 8 μg/ml Protamine Sulfate (Transduction enhancer). Following spinofection, NK92MI cells were further incubated with the lentiviral particles and protamine sulfate for additional 15-18 hours. Medium containing lentivirus and protamine sulfate was replaced with fresh complete medium. Transduction efficiency was detected after 48 hours by mCherry detection on flow cytometer. Using fluorescence activated cell sorter (FACS), mCherry positive NK92MI cells were sorted and cultured to achieve heterogenous pool of EGFR CAR expressing NK92 cells

### Generation of Lair1 Knockout NK92MI and EGFR-CAR NK92MI Cells

Lair1 gene specific gRNA (Table A) was cloned into lentivirus expression plasmid containing Cas9 under EF1α promoter and sgRNA expression under U6 promoter as well as EGFP as detectable marker

To generate Lair1 gRNA and Cas9 containing lentivirus, HEK293T cells were transfected with lentivirus expression plasmid and lentivirus packaging plasmids in OptiMEM medium using Lipofectamin 3000 reagent as per the manufacturer's protocol. Lentivirus containing supernatant (S/N) was collected at 24 and 48 hours after transfection and stored at 4° C. Following 48-hour collection, S/N was filtered using 0.45 μm filters and was ultracentrifuged at 25000 RPM for 1.5 hours to concentrate the lentivirus particles. Lentiviral pellet was resuspended in appropriate volume and stored at −80C until further use. Lentivirus titer was calculated using SupT1 cells by serial dilution and detecting EGFP reporter expression by flow cytometry.

NK92MI and EGFR-CAR NK92MI cells were transduced with Lair1 gRNA and Cas9 expressing lentivirus at MOI 5 by spinofection method (1000 g, 1.5 hrs at 32C) in the presence of 8 μg/ml Polybrene (Transduction enhancer). Following spinofection, NK92MI cells were further incubated with the lentiviral particles and polybrene for additional 4 hours. Medium containing lentivirus and polybrene was replaced with fresh complete medium after 4 hours. Transduction efficiency was detected after 48 hours by EGFP detection on flow cytometer. Using fluorescence activated cell sorter (FACS), EGFP positive NK92MI cells were sorted and cultured to achieve heterogenous pool of transduced NK92 cells with GFP expression.

Alternatively, NK92MI and EGFR-CAR NK92MI were electroporated with Cas9 protein and synthetic Lair1 specific gRNA complex (Ribonucleoprotein, RNP) using Amaxa nucleofector II (Lonza) and A-024 pulse code by following the manufacturer's protocol.

In both lentivirus based and nucleofection-based systems, NK92MI and EGFR CAR NK92MI cells were grown and checked for the loss of Lair1 receptor expression in NK cells. Lair1 knockout clones were sorted using FACS by staining for Lair1 surface expression with fluorescently labelled antibody and sorting for cells with no receptor expression for further functional assays.

### Example 8. Exemplary Protocol to Generate Lair1 Knockout NK92MI and EGFR-CAR NK92MI Cells for Adoptive Therapy

Transducing EGFR CAR In NK92 cells (EGFR-NK92) greatly increased their cytotoxicity against human breast cancer cell line MMDA-MB-231 (FIG. 18A). Finally, to determine whether Lair1 blockade can reverse the inhibitory effect of collagen-I on NK cells, we co-cultured EGFR-NK92 cells with MDA-MB-231 breast cancer cells in the presence of no ECM, collagen-I plus IgG control versus collagen-I plus anti-Lair1 antibody (test). Importantly, collagen-I inhibited CAR NK cell's ability to kill breast cancer cells and this inhibition was reversed by Lair1 blockade (FIG. 181B).

Collectively, these findings demonstrate that ECM proteins are potent regulators of NK cell function in mice and humans. Modification of one or more ECM receptors on NIK cells enhances the therapeutic efficacy and applicability of various NIK cell-based therapies against a broad range of diseases.

TABLE A
ECM receptors on NK cells and gRNA sequences to target CRISPR/Cas9
based genetic modification of these ECM receptors.

| ECM receptor (protein name) | gRNA sequence | SEQ ID NO: |
|---|---|---|
| Lair1 | CCCGGCACACGAAAGTCACA | 18. |
| | TCATTGTGACTGTTGTCCGA | 19. |
| | TACTGAGTCAATGCGGAATC | 20. |
| GPR56 | TAGAGGGCTCTATCAC | 21. |
| | ACCGGATCAACGCCACGGTG | 22. |
| | GAGCTCGCACATGTCCACCG | 23. |
| Integrin a1 | TGCAACAAGTACCTCTTCGG | 24. |
| | GGTAGATCCAACTTTACACA | 25. |
| | GAGGCATTCACGGAAGCCCG | 26. |
| Integrin a2 | TTGTGAGGTCCCCACCATAT | 27. |
| | GGGAATCAGTATTACACAAC | 28. |
| | GTGGCAGTGGATAGGTCAAC | 29. |
| Integrin a3 | GATGGCTACACCAACCGGAC | 30. |
| | CAACCTGGATACCCGATTCC | 31. |
| | GCTACTCGGTCGCCCTCCAT | 32. |
| Integrin a4 | CAACACGCTGTTCGGCTACT | 33. |
| | CGCTTCAGTGATCAATCCCG | 34. |
| | GCTCTCAGTGTCCACGTTGT | 35. |
| Integrin a6 | CTCGGGAGGACAACGTGATC | 36. |
| | TTTCCAGTTATAAGTACCCG | 37. |
| | ACACCGCCCAAAGATGTCTC | 38. |
| Integrin aM | GCCGTAGGTTGGATCCAAAC | 39. |
| | GAGTCATTCGCTACGTCATT | 40. |
| | ATCCTAGTTGTCATCACGGA | 41. |
| Integrin aV | GTGACTGGTCTTCTACCCGC | 42. |
| | CCACGTCTAGGTTGAAGGCG | 43. |
| | TTATGCCAAGGATGATCCAT | 44. |
| Integrin b1 | GCACAACACCAGCTAAGCTC | 45. |
| | AATGTAACCAACCGTAGCAA | 46. |
| | ACAGGTCCATAAGGTAGTAG | 47. |
| Integrin b2 | CGTTCAACGTGACCTTCCGG | 48. |
| | CGGACTCGGTGATCTCGTTG | 49 |
| | CAGCACGGTCTTGTCCACGA | 50. |
| Integrin b3 | CGTGCTGACGCTAACTGACC | 51. |
| | CCCAACATCTGTACCACGCG | 52. |
| | GAGCCGGAGTGCAATCCTCT | 53. |
| Int6grin b5 | TACCAGACCAATCCGTGCAT | 54. |
| | ACCGAGAGGTGATGGACCGT | 55. |
| | CGTCATTCAGATGACACCAC | 56. |
| Elastin binding protein | CGAACCAGCCCTCGATCACC | 57. |
| (EBP) | TACCTAATGACCGCCCCACC | 58. |
| | AGTCGCCGCCAAGTCCCCAA | 59. |
| Neuraminidase 1 (Neu1) | CCAAGTTCATCGCCCTGCGG | 60. |
| | AGCCGGCCTTGTGAGCACAA | 61. |
| | GAATACCAGAGCCCGGTCCA | 62. |
| Cathepsin A | CAGTACTCCGGCTACCTCAA | 63. |
| | TGGAGTACAACCCCTATTCT | 64. |
| | ATAAAACTTGTCATCGGAGT | 65. |
| Galectin 3 | ATGCGAGGCACCACTCCCCC | 66. |
| | TGTTGTTCTCATTGAAGCGT | 67. |
| | CTGCACCTGGAGTCTACCCA | 68. |
| CD44 | GAATACACCTGCAAAGCGGC | 69. |
| | AAGGGCACGTGGTGATTCCC | 70. |
| | TCTGTGCTGTCGGTGATCCA | 71. |
| TLR2 | GACCGCAATGGTATCTGCAA | 72. |
| | TGTAGATGTTACAAGTTCCG | 73. |
| | CACCGATGAAAGTTTGTTTC | 74. |
| TLR4 | TGGTTTAGAAGTCCATCGTT | 75. |
| | AAGGTCCAAGTGCTCTAGAT | 76. |
| | CTTCAATGGTCAAATTGCAC | 77. |
| DDR1 | CACCTATGACGGACATACCG | 78. |
| | GCGAACCAGTCGGGCAACCA | 79 |
| | TGGGAAACACCGACCCTGCG | 80. |
| DDR2 | GGGCTAGGCCAATTGACCGA | 81. |
| | CCGGACAAATCTGGCTACAA | 82. |
| | AAGATCAATTACAGTCGGGA | 83. |
| Plasminogen activator, | GACCACGATCGTGCGCTTGT | 84. |
| Urokinase receptor | GCATTCGAGGTAACGGCTTC | 85 |
| | TTCCACACGGCAATCCCCGT | 86. |

### REFERENCES

1. F. Garrido, N. Aptsiauri, Cancer immune escape: MHC expression in primary tumours versus metastases. Immunology 158, 255-266 (2019).
2. S. Bessoles, C. Grandclement, E. Alari-Pahissa, J. Gehrig, B. Jeevan-Raj, W. Held, Adaptations of Natural Killer Cells to Self-MHC Class I. Front Immunol 5, 349 (2014).
3. E. O. Long, H. S. Kim, D. Liu, M. E. Peterson, S. Rajagopalan, Controlling natural killer cell responses: integration of signals for activation and inhibition. Annu Rev Immunol 31, 227-258 (2013).
4. F. M. Karlhofer, R. K. Ribaudo, W. M. Yokoyama, MHC class I alloantigen specificity of Ly-49+IL-2-activated natural killer cells. Nature 358, 66-70 (1992).
5. M. Bennett, Y. Y. Yu, E. Stoneman, R. M. Rembecki, P. A. Mathew, K. F. Lindahl, V. Kumar, Hybrid resistance: ‘negative’ and ‘positive’ signaling of murine natural killer cells. Semin Immunol 7, 121-127 (1995).
6. V. Kumar, T. George, Y. Y. Yu, J. Liu, M. Bennett, Role of murine NK cells and their receptors in hybrid resistance. Curr Opin Immunol 9, 52-56 (1997).
7. J. O. Manilay, M. Sykes, Natural killer cells and their role in graft rejection. Current opinion in immunology 10, 532-538 (1998).
8. M. Zijlstra, H. Auchincloss, Jr., J. M. Loring, C. M. Chase, P. S. Russell, R. Jaenisch, Skin graft rejection by beta 2-microglobulin-deficient mice. The Journal of experimental medicine 175, 885-893 (1992).
9. L. Riggan, A. G. Freud, T. E. O'Sullivan, True Detective: Unraveling Group 1 Innate Lymphocyte Heterogeneity. Trends Immunol 40, 909-921 (2019).
10. D. K. Sojka, Z. Tian, W. M. Yokoyama, Tissue-resident natural killer cells and their potential diversity. Semin Immunol 26, 127-131 (2014).
11. C. Seillet, G. T. Belz, Differentiation and diversity of subsets in group 1 innate lymphoid cells. Int Immunol 28, 3-11 (2016).
12. V. S. Cortez, T. K. Ulland, L. Cervantes-Barragan, J. K. Bando, M. L. Robinette, Q. Wang, A. J. White, S. Gilfillan, M. Cella, M. Colonna, SMAD4 impedes the conversion of NK cells into ILC1-like cells by curtailing non-canonical TGF-beta signaling. Nat Immunol 18, 995-1003 (2017).
13. Y. Gao, F. Souza-Fonseca-Guimaraes, T. Bald, S. S. Ng, A. Young, S. F. Ngiow, J. Rautela, J. Straube, N. Waddell, S. J. Blake, J. Yan, L. Bartholin, J. S. Lee, E. Vivier, K. Takeda, M. Messaoudene, L. Zitvogel, M. W. L. Teng, G. T. Belz, C. R. Engwerda, N. D. Huntington, K. Nakamura, M. Holzel, M. J. Smyth, Tumor immunoevasion by the conversion of effector NK cells into type 1 innate lymphoid cells. Nat Immunol 18, 1004-1015 (2017).
14. L. L. Lanier, NK cell recognition. Annu Rev Immunol 23, 225-274 (2005).
15. M. D. Bern, B. A. Parikh, L. Yang, D. L. Beckman, J. Poursine-Laurent, W. M. Yokoyama, Inducible down-regulation of MHC class I results in natural killer cell tolerance. The Journal of experimental medicine 216, 99-116 (2019).
16. W. M. Yokoyama, S. Kim, How do natural killer cells find self to achieve tolerance? Immunity 24, 249-257 (2006).
17. P. Brodin, K. Karre, P. Hoglund, NK cell education: not an on-off switch but a tunable rheostat. Trends Immunol 30, 143-149 (2009).
18. J. M. Elliott, W. M. Yokoyama, Unifying concepts of MHC-dependent natural killer cell education. Trends Immunol 32, 364-372 (2011).
19. J. S. Orange, Formation and function of the lytic NK-cell immunological synapse. Nat Rev Immunol 8, 713-725 (2008).
20. R. P. Wallin, V. Screpanti, J. Michaelsson, A. Grandien, H. G. Ljunggren, Regulation of perforin-independent NK cell-mediated cytotoxicity. Eur J Immunol 33, 2727-2735 (2003).
21. R. J. Lebbink, L. Meyaard, Non-MHC ligands for inhibitory immune receptors: novel insights and implications for immune regulation. Mol Immunol 44, 2153-2164 (2007).
22. S. Bauer, V. Groh, J. Wu, A. Steinle, J. H. Phillips, L. L. Lanier, T. Spies, Activation of NK cells and T cells by NKG2D, a receptor for stress-inducible MICA. Science 285, 727-729 (1999).
23. H. R. Smith, J. W. Heusel, I. K. Mehta, S. Kim, B. G. Dorner, O. V. Naidenko, K. Iizuka, H. Furukawa, D. L. Beckman, J. T. Pingel, A. A. Scalzo, D. H. Fremont, W. M. Yokoyama, Recognition of a virus-encoded ligand by a natural killer cell activation receptor. Proceedings of the National Academy of Sciences of the United States of America 99, 8826-8831 (2002).
24. Q. Hammer, T. Ruckert, C. Romagnani, Natural killer cell specificity for viral infections. Nat Immunol 19, 800-808 (2018).
25. K. Karre, H. G. Ljunggren, G. Piontek, R. Kiessling, Selective rejection of H-2-deficient lymphoma variants suggests alternative immune defence strategy. Nature 319, 675-678 (1986).
26. E. Narni-Mancinelli, J. Chaix, A. Fenis, Y. M. Kerdiles, N. Yessaad, A. Reynders, C. Gregoire, H. Luche, S. Ugolini, E. Tomasello, T. Walzer, E. Vivier, Fate mapping analysis of lymphoid cells expressing the NKp46 cell surface receptor. Proceedings of the National Academy of Sciences of the United States of America 108, 18324-18329 (2011).
27. S. K. Tripathy, P. A. Keyel, L. Yang, J. T. Pingel, T. P. Cheng, A. Schneeberger, W. M. Yokoyama, Continuous engagement of a self-specific activation receptor induces NK cell tolerance. The Journal of experimental medicine 205, 1829-1841 (2008).
28. J. M. Elliott, J. A. Wahle, W. M. Yokoyama, MHC class I-deficient natural killer cells acquire a licensed phenotype after transfer into an MHC class I-sufficient environment. The Journal of experimental medicine 207, 2073-2079 (2010).
29. N. T. Joncker, N. Shifrin, F. Delebecque, D. H. Raulet, Mature natural killer cells reset their responsiveness when exposed to an altered MHC environment. The Journal of experimental medicine 207, 2065-2072 (2010).
30. N. D. Huntington, The unconventional expression of IL-15 and its role in NK cell homeostasis. Immunol Cell Biol 92, 210-213 (2014).
31. A. Marcais, S. Viel, M. Grau, T. Henry, J. Marvel, T. Walzer, Regulation of mouse NK cell development and function by cytokines. Front Immunol 4, 450 (2013).
32. T. Hydes, A. Noll, G. Salinas-Riester, M. Abuhilal, T. Armstrong, Z. Hamady, J. Primrose, A. Takhar, L. Walter, S. I. Khakoo, IL-12 and IL-15 induce the expression of CXCR6 and CD49a on peripheral natural killer cells. Immunity, inflammation and disease 6, 34-46 (2018).
33. X. Ni, B. Fu, J. Zhang, R. Sun, Z. Tian, H. Wei, Cytokine-Based Generation of CD49a(+)Eomes(−/+) Natural Killer Cell Subsets. Frontiers in immunology 9, 2126 (2018).
34. V. S. Cortez, L. Cervantes-Barragan, M. L. Robinette, J. K. Bando, Y. Wang, T. L. Geiger, S. Gilfillan, A. Fuchs, E. Vivier, J. C. Sun, M. Cella, M. Colonna, Transforming Growth Factor-beta Signaling Guides the Differentiation of Innate Lymphoid Cells in Salivary Glands. Immunity 44, 1127-1139 (2016).
35. L. Tang, H. Peng, J. Zhou, Y. Chen, H. Wei, R. Sun, W. M. Yokoyama, Z. Tian, Differential phenotypic and functional properties of liver-resident NK cells and mucosal ILC1s. J Autoimmun 67, 29-35 (2016).
36. D. K. Sojka, B. Plougastel-Douglas, L. Yang, M. A. Pak-Wittel, M. N. Artyomov, Y. Ivanova, C. Zhong, J. M. Chase, P. B. Rothman, J. Yu, J. K. Riley, J. Zhu, Z. Tian, W. M. Yokoyama, Tissue-resident natural killer (NK) cells are cell lineages distinct from thymic and conventional splenic NK cells. eLife 3, e01659 (2014).
37. S. Viel, A. Marcais, F. S. Guimaraes, R. Loftus, J. Rabilloud, M. Grau, S. Degouve, S. Djebali, A. Sanlaville, E. Charrier, J. Bienvenu, J. C. Marie, C. Caux, J. Marvel, L. Town, N. D. Huntington, L. Bartholin, D. Finlay, M. J. Smyth, T. Walzer, TGF-beta inhibits the activation and functions of NK cells by repressing the mTOR pathway. Sci Signal 9, ra19 (2016).
38. L. K. Mackay, M. Minnich, N. A. Kragten, Y. Liao, B. Nota, C. Seillet, A. Zaid, K. Man, S. Preston, D. Freestone, A. Braun, E. Wynne-Jones, F. M. Behr, R. Stark, D. G. Pellicci, D. I. Godfrey, G. T. Belz, M. Pellegrini, T. Gebhardt, M. Busslinger, W. Shi, F. R. Carbone, R. A. van Lier, A. Kallies, K. P. van Gisbergen, Hobit and Blimp1 instruct a universal transcriptional program of tissue residency in lymphocytes. Science (New York, N.Y 352, 459-463 (2016).
39. R. J. Lebbink, T. de Ruiter, G. J. Kaptijn, D. G. Bihan, C. A. Jansen, P. J. Lenting, L. Meyaard, Mouse leukocyte-associated Ig-like receptor-1 (mLAIR-1) functions as an inhibitory collagen-binding receptor on immune cells. Int Immunol 19, 1011-1019 (2007).
40. L. Meyaard, The inhibitory collagen receptor LAIR-1 (CD305). J Leukoc Biol 83, 799-803 (2008).
41. A. Corcoran, T. G. Cotter, Redox regulation of protein kinases. FEBS J 280, 1944-1965 (2013).
42. B. Diop-Frimpong, V. P. Chauhan, S. Krane, Y. Boucher, R. K. Jain, Losartan inhibits collagen I synthesis and improves the distribution and efficacy of nanotherapeutics in tumors. Proceedings of the National Academy of Sciences of the United States of America 108, 2909-2914 (2011).
43. G. Xiong, R. L. Stewart, J. Chen, T. Gao, T. L. Scott, L. M. Samayoa, K. O'Connor, A. N. Lane, R. Xu, Collagen prolyl 4-hydroxylase 1 is essential for HIF-1alpha stabilization and TNBC chemoresistance. Nat Commun 9, 4456 (2018).
44. E. Narni-Mancinelli, S. Ugolini, E. Vivier, Tuning the threshold of natural killer cell responses. Curr Opin Immunol 25, 53-58 (2013).
45. F. D. Shi, H. G. Ljunggren, A. La Cava, L. Van Kaer, Organ-specific features of natural killer cells. Nat Rev Immunol 11, 658-671 (2011).
46. J. P. Bottcher, E. Bonavita, P. Chakravarty, H. Blees, M. Cabeza-Cabrerizo, S. Sammicheli, N. C. Rogers, E. Sahai, S. Zelenay, C. Reis e Sousa, NK Cells Stimulate Recruitment of cDC1 into the Tumor Microenvironment Promoting Cancer Immune Control. Cell 172, 1022-1037 e1014 (2018).
47. M. A. Degli-Esposti, M. J. Smyth, Close encounters of different kinds: dendritic cells and NK cells take centre stage. Nat Rev Immunol 5, 112-124 (2005).
48. G. Gasteiger, A. Y. Rudensky, Interactions between innate and adaptive lymphocytes. Nat Rev Immunol 14, 631-639 (2014).
49. P. Kalinski, A. Giermasz, Y. Nakamura, P. Basse, W. J. Storkus, J. M. Kirkwood, R. B. Mailliard, Helper role of NK cells during the induction of anticancer responses by dendritic cells. Mol Immunol 42, 535-539 (2005).
50. R. B. Mailliard, Y. I. Son, R. Redlinger, P. T. Coates, A. Giermasz, P. A. Morel, W. J. Storkus, P. Kalinski, Dendritic cells mediate NK cell help for Th1 and CTL responses: two-signal requirement for the induction of NK cell helper function. J Immunol 171, 2366-2373 (2003).
51. A. Martin-Fontecha, L. L. Thomsen, S. Brett, C. Gerard, M. Lipp, A. Lanzavecchia, F. Sallusto, Induced recruitment of NK cells to lymph nodes provides IFN-gamma for T(H)1 priming. Nature immunology 5, 1260-1265 (2004).
52. R. Mocikat, H. Braumuller, A. Gumy, O. Egeter, H. Ziegler, U. Reusch, A. Bubeck, J. Louis, R. Mailhammer, G. Riethmuller, U. Koszinowski, M. Rocken, Natural killer cells activated by MHC class I(low) targets prime dendritic cells to induce protective CD8 T cell responses. Immunity 19, 561-569 (2003).
53. J. P. Bottcher, E. Bonavita, P. Chakravarty, H. Blees, M. Cabeza-Cabrerizo, S. Sammicheli, N. C. Rogers, E. Sahai, S. Zelenay, E. S. C. Reis, NK Cells Stimulate Recruitment of cDC1 into the Tumor Microenvironment Promoting Cancer Immune Control. Cell, (2018).
54. A. Le Moine, M. Goldman, D. Abramowicz, Multiple pathways to allograft rejection. Transplantation 73, 1373-1381 (2002).
55. M. Wise, D. Zelenika, F. Bemelman, D. Latinne, H. Bazin, S. Cobbold, H. Waldmann, CD4 T cells can reject major histocompatibility complex class I-incompatible skin grafts. Eur J Immunol 29, 156-167 (1999).
56. J. Marino, J. Paster, G. Benichou, Allorecognition by T Lymphocytes and Allograft Rejection. Front Immunol 7, 582 (2016).
57. G. Habif, A. Crinier, P. Andre, E. Vivier, E. Narni-Mancinelli, Targeting natural killer cells in solid tumors. Cell Mol Immunol 16, 415-422 (2019).
58. G. Nayyar, Y. Chu, M. S. Cairo, Overcoming Resistance to Natural Killer Cell Based Immunotherapies for Solid Tumors. Front Oncol 9, 51 (2019).
59. M. H. Sandel, F. M. Speetjens, A. G. Menon, P. A. Albertsson, P. H. Basse, M. Hokland, J. F. Nagelkerke, R. A. Tollenaar, C. J. van de Velde, P. J. Kuppen, Natural killer cells infiltrating colorectal cancer and MHC class I expression. Mol Immunol 42, 541-546 (2005).
60. J. J. Trombetta, D. Gennert, D. Lu, R. Satija, A. K. Shalek, A. Regev, Preparation of Single-Cell RNA-Seq Libraries for Next Generation Sequencing. Curr Protoc Mol Biol 107, 4 22 21-17 (2014).
61. Kundu S, Gurney M, O'Dwyer M. Generating natural killer cells for adoptive transfer: expanding horizons. Cytotherapy. 2021; 23(7):559-66.
62. Tang X, Yang L, Li Z, Nalin A P, Dai H, Xu T, Yin J, You F, Zhu M, Shen W, Chen G, Zhu X, Wu D, Yu J. First-in-man clinical trial of CAR NK-92 cells: safety test of CD33-CAR NK-92 cells in patients with relapsed and refractory acute myeloid leukemia. American journal of cancer research. 2018; 8(6):1083-9.
63. Gong Y, Klein Wolterink R G J, Wang J, Bos G M J, Germeraad W T V. Chimeric antigen receptor natural killer (CAR-NK) cell design and engineering for cancer therapy. Journal of hematology & oncology. 2021; 14(1):73.
64. Romee R, Rosario M, Berrien-Elliott M M, Wagner J A, Jewell B A, Schappe T, Leong J W, Abdel-Latif S, Schneider S E, Willey S, Neal C C, Yu L, Oh S T, Lee Y S, Mulder A, Claas F, Cooper M A, Fehniger T A. Cytokine-induced memory-like natural killer cells exhibit enhanced responses against myeloid leukemia. Science translational medicine. 2016; 8(357):357ra123.
65. Shankar K, Capitini C M, Saha K. Genome engineering of induced pluripotent stem cells to manufacture natural killer cell therapies. Stem cell research & therapy. 2020; 11(1):234.
66. Jayaraman J, Mellody M P, Hou A J, Desai R P, Fung A W, Pham A H T, Chen Y Y, Zhao W. CAR-T design: Elements and their synergistic function. EBioMedicine. 2020; 58:102931.
67. Rafiq S, Hackett C S, Brentjens R J. Engineering strategies to overcome the current roadblocks in CAR T cell therapy. Nature reviews Clinical oncology. 2020; 17(3):147-67.
68. Kumar S. Natural killer cell cytotoxicity and its regulation by inhibitory receptors. Immunology. 2018; 154(3):383-93.
69. Chen L, Flies D B. Molecular mechanisms of T cell co-stimulation and co-inhibition. Nature reviews Immunology. 2013; 13(4):227-42.
70. Seif M, Einsele H, Loffler J. CAR T Cells Beyond Cancer: Hope for Immunomodulatory Therapy of Infectious Diseases. Frontiers in immunology. 2019; 10:2711.
71. Aghajanian H, Rurik J G, Epstein J A. CAR-based therapies: opportunities for immuno-medicine beyond cancer. Nature metabolism. 2022; 4(2):163-9.
72. Morgan M A, Buning H, Sauer M, Schambach A. Use of Cell and Genome Modification Technologies to Generate Improved “Off-the-Shelf” CAR T and CAR NK Cells. Frontiers in immunology. 2020; 11:1965.
73. Shakirova K M, Ovchinnikova V Y, Dashinimaev E B. Cell Reprogramming With CRISPR/Cas9 Based Transcriptional Regulation Systems. Frontiers in bioengineering and biotechnology. 2020; 8:882
74. Purdy A K, Campbell K S. Introduction of shRNAs into human NK-like cell lines with retrovirus. Methods in molecular biology. 2010; 612:223-31.
75. Huang R S, Lai M C, Shih H A, Lin S. A robust platform for expansion and genome editing of primary human natural killer cells. The Journal of experimental medicine. 2021; 218(3).
76. Meyaard L, Adema G J, Chang C, Woollatt E, Sutherland G R, Lanier L L, Phillips J H. LAIR-1, a novel inhibitory receptor expressed on human mononuclear leukocytes. Immunity. 1997; 7(2):283-90.
77. Meyaard L. The inhibitory collagen receptor LAIR-1 (CD305). Journal of leukocyte biology. 2008; 83(4):799-803.
78. Bachmann M, Kukkurainen S, Hytonen V P, Wehrle-Haller B. Cell Adhesion by Integrins. Physiological reviews. 2019; 99(4):1655-99.
79. Dustin M L. Integrins and Their Role in Immune Cell Adhesion. Cell. 2019; 177(3):499-501.
80. Chang G W, Hsiao C C, Peng Y M, Vieira Braga F A, Kragten N A, Remmerswaal E B, van de Garde M D, Straussberg R, Konig G M, Kostenis E, Knauper V, Meyaard L, van Lier R A, van Gisbergen K P, Lin H H, Hamann J. The Adhesion G Protein-Coupled Receptor GPR56/ADGRG1 Is an Inhibitory Receptor on Human NK Cells. Cell reports. 2016; 15(8):1757-70.
81. Sague S L, Tato C, Pure E, Hunter C A. The regulation and activation of CD44 by natural killer (NK) cells and its role in the production of IFN-gamma. Journal of interferon & cytokine research: the official journal of the International Society for Interferon and Cytokine Research. 2004; 24(5):301-9.
82. Noh J Y, Yoon S R, Kim T D, Choi I, Jung H. Toll-Like Receptors in Natural Killer Cells and Their Application for Immunotherapy. Journal of immunology research. 2020; 2020:2045860.
83. Zhang C, Oberoi P, Oelsner S, Waldmann A, Lindner A, Tonn T, et al., Chimeric antigen receptor-engineered NK-92 cells: An off-the-Shelf cellular therapeutic for targeted elimination of cancer cells and induction of protective antitumor immunity. Front Immunol (2017) 8:533.
84. Yao, X., Matosevic, S. Cryopreservation of NK and T Cells Without DMSO for Adoptive Cell-Based Immunotherapy. BioDrugs 35, 529-545 (2021).
85. Min B, Choi H, Her J H, Jung M Y, Kim H J, Jung M Y, Lee E K, Cho S Y, Hwang Y K, Shin E C. Optimization of Large-Scale Expansion and Cryopreservation of Human Natural Killer Cells for Anti-Tumor Therapy. Immune Netw. 2018 Aug. 21; 18(4):e31.
86. Schmidt P, Raftery M J, Pecher G. Engineering NK Cells for CAR Therapy-Recent Advances in Gene Transfer Methodology. Front Immunol. 2021 Jan. 7; 11:611163.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing a population of modified Natural Killer (NK) cells, the method comprising:
obtaining a first population comprising NK cells;
modifying the first population of NK cells to specifically increase, reduce, or eliminate expression of at least one extracellular matrix (ECM) receptor; and
optionally maintaining the modified cells in culture under conditions and for a time sufficient for the cells to proliferate,
thereby providing a population of modified NK cells.

2. The method of claim 1, wherein modifying the first population of NK cells to specifically increase expression of at least one ECM receptor comprises introducing into the cells at least one expression vector comprising a nucleic acid encoding an ECM receptor polypeptide, optionally wherein the expression vector is a viral vector.

3. The method of claim 1, wherein modifying the first population of NK cells to specifically reduce or eliminate expression of at least one ECM receptor comprises using genome engineering to disrupt at least one ECM receptor gene, optionally wherein the genome engineering used clustered regularly interspaced short palindromic repeats (CRISPR)/CRISPR-associated protein (Cas)-based gene editing, zinc-finger nucleases (ZFN), or transcription activator-like nucleases (TALENs)-based systems to target the ECM receptor gene.

4. The method of claim 1, wherein modifying the first population of NK cells to specifically reduce or eliminate expression of at least one ECM receptor comprises contacting the cells with one or more inhibitory nucleic acids, optionally antisense oligonucleotides, LNAs, RNA interference (RNAi), or shRNA, that specifically inhibits expression of the at least one ECM receptor.

5. The method of claim 1, wherein the at least one ECM receptor is selected from the group consisting of LAIR1, GPR56, ITGB1, ITGA2, ITGAM, ITGB2, ITGA6, ITGA4, NEU1, EBP, ITGB3, PLAUR, DDR1, DDR2, ITGA3, ITGA1, ITGAV, CTSA, LGALS3, and ITGB5, and combinations of two or more thereof.

6. The method of claim 5, wherein the first population of NK cells is obtained from autologous and/or allogeneic peripheral blood mononuclear cells (PBMCs), umbilical cord blood (UCB), embryonic stem cells (ESC), induced pluripotent stem cells (iPSC), or an immortalized NK cell line.

7. The method of claim 6, wherein the cell line is NK-92 or a derivative thereof selected from as NKL and NK101; or YT or a derivative thereof selected from HANK-1, KHYG-1, NK-YS, and NKG.

8. The method of claim 7, wherein the cells are modified to specifically reduce or eliminate expression of at least one ECM receptor, and are further modified to express a chimeric antigen receptor (CAR).

9. A population of modified NK cells prepared by the method of claim 1.

10. (canceled)

11. The population of modified NK cells of claim 9, wherein the NK cells have been modified to specifically increase expression of at least one ECM receptor by introducing into the cells at least one expression vector comprising a nucleic acid encoding an ECM receptor polypeptide, optionally wherein the expression vector is a viral vector.

12. The population of modified NK cells of claim 9, wherein the NK cells have been modified to specifically reduce or eliminate expression of at least one ECM receptor using genome engineering to disrupt at least one ECM receptor gene, optionally wherein the genome engineering used clustered regularly interspaced short palindromic repeats (CRISPR)/CRISPR-associated protein (Cas)-based gene editing, zinc-finger nucleases (ZFN), or transcription activator-like nucleases (TALENs)-based systems to target the ECM receptor gene.

13. The population of modified NK cells of claim 9, wherein the NK cells have been modified to specifically reduce or eliminate expression of at least one ECM receptor by contacting the cells with one or more inhibitory nucleic acids, optionally antisense oligonucleotides, LNAs, RNA interference (RNAi), or shRNA, that specifically inhibits expression of the at least one ECM receptor.

14. The population of modified NK cells of claim 11, wherein the at least one ECM receptor is selected from the group consisting of LAIR1, GPR56, ITGB1, ITGA2, ITGAM, ITGB2, ITGA6, ITGA4, NEU1, EBP, ITGB3, PLAUR, DDR1, DDR2, ITGA3, ITGA1, ITGAV, CTSA, LGALS3, and ITGB5, and combinations of two or more thereof.

15. The population of modified NK cells of claim 11, wherein the modified NK cells were obtained from autologous and/or allogeneic peripheral blood mononuclear cells (PBMCs), umbilical cord blood (UCB), embryonic stem cells (ESC), induced pluripotent stem cells (iPSC), or an immortalized NK cell line.

16. The population of modified NK cells of claim 9, the cell line is NK-92 or a derivative thereof selected from as NKL and NK101; or YT or a derivative thereof selected from HANK-1, KHYG-1, NK-YS, and NKG.

17. The population of modified NK cells of claim 9, wherein the cells are modified to specifically reduce or eliminate expression of at least one ECM receptor, and are further modified to express a chimeric antigen receptor (CAR).

18. (canceled)

19. (canceled)

20. A method of treating a subject who has cancer, the method comprising administering to the subject a therapeutically effective amount of the modified NK cells of claim 9.

21. The method of claim 20, wherein the NK cells were obtained from the subject who has cancer.

22. The method of claim 20, wherein the cancer is colon cancer, ovarian cancer, prostate cancer, lymphoid malignancies, myeloma, renal cell carcinoma, breast cancer, or malignant glioma.

23. The population of modified NK cells of claim 9, wherein the cells are modified to specifically increase expression of at least one ECM receptor.

24. (canceled)

25. (canceled)

26. (canceled)

27. (canceled)
